SEQUENCE LISTING (95 sequences)

SEQ ID NO: 1 - protein, 1378 aa, Streptococcus pyogenes
VAPKKKRKVMDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKKKKLKL

SEQ ID NO: 2 - protein, 8 aa, Artificial Sequence
  synthesized sequence
APKKKRKV

SEQ ID NO: 3 - protein, 6 aa, Artificial Sequence
  synthesized sequence
KKKKLK

SEQ ID NO: 4 - protein, 10 aa, Artificial Sequence
  synthesized sequence
HHHHHHHHHH

SEQ ID NO: 5 - DNA, 50 bp, Artificial Sequence
  synthesized sequence
atatatgagtaaacttggtctgacagaattcctccattttcttctgctat

SEQ ID NO: 6 - DNA, 35 bp, Artificial Sequence
  synthesized sequence
tgcggccgcgaattcgattacgaatgccgtctccc

SEQ ID NO: 7 - DNA, 3290 bp, Artificial Sequence
  synthesized sequence
gaattcgcggccgcacgcgtccatggggatccccgcgggtcgacctcgagagttacgctagggataacagggtaatataggagctccagtcggcttaaaccagttttcgctggtgcgaaaaaagagtgtcttgtgacacctaaattcaaaatctatcggtcagatttataccgatttgattttatatattcttgaataacatacgccgagttatcacataaaagcgggaaccaatcataaaatttaaacttcattgcataatccattaaactcttaaattctacgattccttgttcatcaataaactcaatcatttctttaattaatttatatctatctgttgttgttttctttaataattcattaacatctacaccgccataaactatcatatcttctttttgatatttaaatttattaggatcgtccatgtgaagcatatatctcacaagacctttcacacttcctgcaatctgcggaatagtcgcattcaattcttctgttaattatttttatctgttcataagatttattaccctcatacatcactagaatatgataatgctcttttttcatcctaccttctgtatcagtatccctatcatgtaatggagacactacaaattgaatgtgtaactcttttaaatactctaaccactcggcttttgctgattctggatataaaacaaatgtccaattacgtcctcttgaatttttcttgttttcagtttcttttattacattttcgctcatgatataataacggtgctaatacacttaacaaaatttagtcatagataggcagcatgccagtgctgtctatctttttttgtttaaaatgcaccgtattcctcctttgcatatttttttattagaataccggttgcatctgatttgctaatattatatttttctttgattctatttaatatctcattttcttctgttgtaagtcttaaagtaacagcaacttttttctcttcttttctatctacaactatcactgtacctcccaacatctgtttttttcactttaacataaaaaacaaccttttaacattaaaaacccaatatttatttatttgtttggacaatggacactggacacctaggggggaggtcgtagtacccccctatgttttctcccctaaataaccccaaaaatctaagaaaaaaagacctcaaaaaggtctttaattaacatctcaaatttcgcatttattccaatttcctttttgcgtgtgatgcgagctcatcggctccgtcgatactatgttatacgccaactttcaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtgactgcagtgagatctggtaatgactctctagcttgaggcatcaaataaaacgaaaggctcagtcgaaagactgggcctttcgttttatctgttgtttgtcggtgaacgctctcctgagtaggacaaatccgccgctctagctaagcagaaggccatcctgacggatggcctttttgcgtttctacaaactcttgttaactctagagctgcctgccgcgtttcggtgatgaagatcttcccgatgattaattaattcagaacgctcggttgccgccgggcgttttttatgaagcttcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgaca

SEQ ID NO: 8 - DNA, 4204 bp, Artificial Sequence
  synthesized sequence
gcggccgcacgcgtccatggggatccccgcgggtcgacctcgagagttacgctagggataacagggtaatataggagctccagtcggcttaaaccagttttcgctggtgcgaaaaaagagtgtcttgtgacacctaaattcaaaatctatcggtcagatttataccgatttgattttatatattcttgaataacatacgccgagttatcacataaaagcgggaaccaatcataaaatttaaacttcattgcataatccattaaactcttaaattctacgattccttgttcatcaataaactcaatcatttctttaattaatttatatctatctgttgttgttttctttaataattcattaacatctacaccgccataaactatcatatcttctttttgatatttaaatttattaggatcgtccatgtgaagcatatatctcacaagacctttcacacttcctgcaatctgcggaatagtcgcattcaattcttctgttaattatttttatctgttcataagatttattaccctcatacatcactagaatatgataatgctcttttttcatcctaccttctgtatcagtatccctatcatgtaatggagacactacaaattgaatgtgtaactcttttaaatactctaaccactcggcttttgctgattctggatataaaacaaatgtccaattacgtcctcttgaatttttcttgttttcagtttcttttattacattttcgctcatgatataataacggtgctaatacacttaacaaaatttagtcatagataggcagcatgccagtgctgtctatctttttttgtttaaaatgcaccgtattcctcctttgcatatttttttattagaataccggttgcatctgatttgctaatattatatttttctttgattctatttaatatctcattttcttctgttgtaagtcttaaagtaacagcaacttttttctcttcttttctatctacaactatcactgtacctcccaacatctgtttttttcactttaacataaaaaacaaccttttaacattaaaaacccaatatttatttatttgtttggacaatggacactggacacctaggggggaggtcgtagtacccccctatgttttctcccctaaataaccccaaaaatctaagaaaaaaagacctcaaaaaggtctttaattaacatctcaaatttcgcatttattccaatttcctttttgcgtgtgatgcgagctcatcggctccgtcgatactatgttatacgccaactttcaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtgactgcagtgagatctggtaatgactctctagcttgaggcatcaaataaaacgaaaggctcagtcgaaagactgggcctttcgttttatctgttgtttgtcggtgaacgctctcctgagtaggacaaatccgccgctctagctaagcagaaggccatcctgacggatggcctttttgcgtttctacaaactcttgttaactctagagctgcctgccgcgtttcggtgatgaagatcttcccgatgattaattaattcagaacgctcggttgccgccgggcgttttttatgaagcttcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatggaattc

SEQ ID NO: 9 - DNA, 35 bp, Artificial Sequence
  synthesized sequence
gggagacggcattcgtaatcgaattcgcggccgca

SEQ ID NO: 10 - DNA, 50 bp, Artificial Sequence
  synthesized sequence
atagcagaagaaaatggaggaattctgtcagaccaagtttactcatatat

SEQ ID NO: 11 - DNA, 23 bp, Artificial Sequence
  synthesized sequence
ccgactggagctcctatattacc

SEQ ID NO: 12 - DNA, 20 bp, Artificial Sequence
  synthesized sequence
gctgtggcgatctgtattcc

SEQ ID NO: 13 - DNA, 22 bp, Artificial Sequence
  synthesized sequence
gtcttttaagtaagtctactct

SEQ ID NO: 14 - DNA, 20 bp, Artificial Sequence
  synthesized sequence
ccaaagcgattttaagcgcg

SEQ ID NO: 15 - DNA, 20 bp, Artificial Sequence
  synthesized sequence
cctggcacgtggtaattctc

SEQ ID NO: 16 - DNA, 20 bp, Artificial Sequence
  synthesized sequence
ggatttcctcaaatctgacg

SEQ ID NO: 17 - DNA, 21 bp, Artificial Sequence
  synthesized sequence
gtagaaacgcgccaaattacg

SEQ ID NO: 18 - DNA, 20 bp, Artificial Sequence
  synthesized sequence
gctggtggttgctaaagtcg

SEQ ID NO: 19 - DNA, 20 bp, Artificial Sequence
  synthesized sequence
ggacgcaaccctcattcatc

SEQ ID NO: 20 - DNA, 380 bp, Artificial Sequence
  synthesized sequence
gggtgaagtggtcaagacctcactaggcaccttaaaaatagcgcaccctgaagaagatttatttgaggtagcccttgcctacctagcttccaagaaagatatcctaacagcacaagagcggaaagatgttttgttctacatccagaacaacctctgctaaaattcctgaaaaattttgcaaaaagttgttgactttatctacaaggtgtggcataatgtgtggatggctgcatctatttatattgttttagagctagaaatagcaagttaaaataaggctagtccgttatcaacttgaaaaagtggcaccgagtcggtgcctagtaaatgcacgagcaacatcttttgttgctcagtgcattttttattttgtacactag

SEQ ID NO: 21 - DNA, 1549 bp, Artificial Sequence
  synthesized sequence
gctgataaacagctgacatcaactaaaagtttcattaaatactttgaaaaaagttgttgacttaaaagaagctaaatgttatagtaattgtacagaatagtcttttaagtaagtctactctgaatttttttaaaaggagagggtaaagagtgagaagcaaaaaattgtggatcagcttgttgtttgcgttaacgttaatctttacgatggcgttcagcaacatgtctgcgcaggctgctgaagaagcaaaagaaaaatatttaattggctttaatgagcaggaagctgtcagtgagtttgtagaacaagtagaggcaaatgacaatgtcgccattctctctgaggaagaggaagtcgaaattgaattgcttcatgaatttgaaacgattcctgttttatccgttgagttaagcccagaagatgtggacgcgcttgaactcgatccagcgatttcttatattgaagaggatgcagaagtaacgacaatggcgcaatcggtaccatggggaattagccgtgtgcaagccccagctgcccataaccgtggattgacaggttctggtgtaaaagttgctgtcctcgatacaggtatttccactcatccagacttaaatattcgtggtggcgctagctttgtaccaggggaaccatccactcaagatgggaatgggcatggcacgcatgtggctgggacgattgctgctttaaacaattcgattggcgttcttggcgtagcaccgaacgcggaactatacgctgttaaagtattaggggcgagcggttcaggttcggtcagctcgattgcccaaggattggaatgggcagggaacaatgttatgcacgttgctaatttgagtttaggactgcaggcaccaagtgccacacttgagcaagctgttaatagcgcgacttctagaggcgttcttgttgtagcggcatctgggaattcaggtgcaggctcaatcagctatccggcccgttatgcgaacgcaatggcagtcggagctactgaccaaaacaacaaccgcgccagcttttcacagtatggcgcagggcttgacattgtcgcaccaggtgtaaacgtgcagagcacatacccaggttcaacgtatgccagcttaaacggtacatcgatggctactcctcatgttgcaggtgcagcagcccttgttaaacaaaagaacccatcttggtccaatgtacaaatccgcaatcatctaaagaatacggcaacgagcttaggaagcacgaacttgtatggaagcggacttgtcaatgcagaagcggcaacacgttaagcttacataaaaaaccggccttggccccgccggttttttattatttttcttcctccgcatgttcaatccgctccataatcgacggatggctccctctgaaaattttaacgagaaacggcgggttgacccggctcagtcccgtaacggccaagtcctgaaacgtctcaatcgccgcttcccggtttccggtcagctcaatgccgtaacggtcggcggcgttttcctgataccgggagacggcattcgtaatc

SEQ ID NO: 22 - DNA, 600 bp, Bacillus subtilus
gaagctgaatgagatttcttaaggctttgccgcggcgagctgaagtacagtacgattgccttgatcgaacactggaaacacaagagaacgtaaacctgaacatccgcgtaaacgtcaaggaagttgccacttggggagtaaacacttgcatcatctcgctgaaaggcctagacaatgcggatgacagattcgtattgccggaagtaaacacagcgcttgctctgtttccgttgtcgattgcagctgattgcttactaatgctgccctgcatctcggttgtgatgtcgataagcagcgtaatgaagagtgttactgtggagtaagatgtttaacccctctggatattttagccgaaggggtttttagtatagatatttgatttaaattacaggagacttcattcatttaaccaaagtcattgctttcttaatcactaaagtttatttgaaaataatctcttgatttaatttcctcgaagagattttttgtcaatctattaggcatcagaatttttataacataatggaccgtctttttgacgttttgtttatagaacaagaaaatattcaaaacataagtggaaaattaggggtgagctcc

SEQ ID NO: 23 - DNA, 600 bp, Bacillus subtilus
ggtccttcctataagcaggaaggttttttaatgaaatatttttttattagtaggtaaattcatcaaatgttctgctatgctccaaatgtacacctttccgtaagggcaaagtcaaatgtgaaaaaaaactgttccaaagttaatcagaaatttattttcatattgtatccttcggtatcaagtgaaaggagcatcatatgaaaacattatggaaagtcctcaaaattgtttttgtcagcttggctgctttggttttgcttgtatccgtctcggtatttatttatcaccatttccagctaaataaggaggcggcactgctgaaaggtaaaggcacagtagtcgatgttgacggtaaaaagatgaatgtgtatcaagagggaagcgggaaggatacgtttgtgtttatgtccggttcgggtattgctgcgcctgcttatgaaatgaaggggctgtacagcaagttttcaaaagaaaataagattgctgttgtagatcgggcaggttatggatacagtgaagtgtctcacgatgacagagatattgatacggtattggaacagacgaggaaagcgcttatgaaaagcggaaataagcctcctt

SEQ ID NO: 24 - DNA, 793 bp, Staphylococcus aureus
ttttaaaatatatatttatgttacagtaatattgacttttaaaaaaggattgattctaatgaagaaagcagacaagtaagcctcctaaattcactttagataaaaatttaggaggcatatcaaatgaactttaataaaattgatttagacaattggaagagaaaagagatatttaatcattatttgaaccaacaaacgacttttagtataaccacagaaattgatattagtgttttataccgaaacataaaacaagaaggatataaattttaccctgcatttattttcttagtgacaagggtgataaactcaaatacagcttttagaactggttacaatagcgacggagagttaggttattgggataagttagagccactttatacaatttttgatggtgtatctaaaacattctctggtatttggactcctgtaaagaatgacttcaaagagttttatgatttatacctttctgatgtagagaaatataatggttcggggaaattgtttcccaaaacacctatacctgaaaatgctttttctctttctattattccatggacttcatttactgggtttaacttaaatatcaataataatagtaattaccttctacccattattacagcaggaaaattcattaataaaggtaattcaatatatttaccgctatctttacaggtacatcattctgtttgtgatggttatcatgcaggattgtttatgaactctattcaggaattgtcagataggcctaatgactggcttttataatatgaggtcgacgcatgcg

SEQ ID NO: 25 - DNA, 5801 bp, Enterococcus faecalis
ctagtagccctctttaaagggcttttatgtttattttgagaaagatataaaatcaatatatcccttttctccgatttttacaacggcattgtaggactttctatctttcgttttgattccttttaccagggtttcttttccctctagtaattcttttacatttgttttggtgagttttttctttctaaaatgttcagctaaagtaaacttacattcaggataatttgaacaaccataaaacgatttttttaatacaatattgttgccacacttaggacattttcctacaatacttttttctgcttctttttctttctgttcctggtaatcagaaaaatttagtttttctatatcgttaggtacagcttccagtaaatgaacaatgaattttttgatattcgtaataaagttctcttgattgccttctcttttaccgattttttttaaatacgtttcccatttagccgtcatttcagcactcgttaaaaggtgctgactttcaactgcctggcacaataattttcctttttcagttacaacaagcttattcttaatcacttggatatattctttttgtttcaaggcttcaataatgcttgctcttgtcgcttctgttccaatcccctcaacttcttgtaaaatcttgattgcttcttcatcatcaaccgttttattcgccgttttcatagcagttaataatgtaccctctgtaaaagctttcggtggttgtgtttctttttcggcactcttaacgtcaacctcggcatgttctccaatgataaccaagggtaacgtttgaacgtcctcttcttcttctttggtttgttgtttgaaaagaattttccaaccttcttgctttggtgtcttgcctattgattgaaaaagtaagtcggctactttggtttgtatcttagtttcttcatacaaataatcaggtagaaacatggcaacggtcgttttaacgactaaagcatatatttttcgttgtaaatcgtccattttcgctaatgcagattcggtaggtacttgttttgttgggataatggcgtggtgttcctgtaccttactaccgtccacataacgctttctaggctctgtttgaaccatttcaagatcaagtcctaaaaaaccgctatatttgccaaaattcgcttttaaataagcaaattcgttctctgtaataaatggtgtatctgttcttggataactcaataattttgcttcatacagtccttgcatagcttttaaagtttggctcgctgtcgctttataaagctgattaacttttgattgcaaactacttaaagaaaacaaactcggactattcgttttcttctctttggtttgaacatcagcaattatcccctcttgattgcctattttagcttgttcagaagaaacaaaagctaaaagctcctcttgggttttaaagcgctgtgtggggcttataacgcccttaaatgacccttggtttacttttatactagcttccacctcgaaaaaaggttcttttctaaagttttctatggcttcctggcgctgaaaaataagatataaggtgggcgtttgaacacgtcctagtgaaaatgtaccttgtacgcccttctgctgtaaatttaacgtatacaaagggcttgcattcatgccgatcaaccaatcggcaatttggcgtgtttgcgcttcttgataaaagggatagtaattcattcctggttgcaaattttgaaaaccgctacggatcacatctttttctaaactattgatccatagtcttttatacgttttatctttagaaaaggcatttgctttatgaatgatcgaccaggcaatgttttcgccttctctgtcgctatctgttgcgacaatgattgtatttgcttgttttaaaagttcagcaacaattttaaactgcttttttttatctgttgccacttcaaaatcgtatcgatcaggaaaaatcggcaatgattcaagtttccaattttgccacttttcgtcataatgacctggttctgctaactcgactaaatgcccaaaaccaaacgtgataaacgtttcatctgcaaagattgggtctttgatctcaaaataacagtcttttttggtgctttgttttaaagcacttgcataggctaatgcctggcttggtttttcagctaaaataaccgtactcattaactatccctcttttcattgttttttctttgatcgactgtcacgttgtatcttgatcgataccttctaaacgttcggcgattaattccagtttgttcttcaacttctttatcggataaaccgttcaaaaacaaatcgaaagcgtgttgtaaacgtggatcattttctttgaatttcagttgtcgccctttgaattttccttttttcttagcaatttcaattccttgggcttggcgttctttaattcgtttgcgttcagattccgcttggtacttatacaattcaatcactaaattattaatcagccgtcttaaattttcatcttcaataccattcattgagggtaaatttaagacttccagggttgcccccttaatttgaatttgattcatcaattctgttaattctttattatttcgtcctaatcgatctaattcagtaacaacaactatatccccttcacgaatatagttaagcatagcttgtaattgtgggcgttcgaccgattgaccgcttgctttgtctgaaaagaccttagaaacgccctctaacgctttcagttgtctatctaagttctgttctttgctactgacacgtgcataccctactttagtcatgttaaaaaacacctcccttgttaagaaaaaaataaattggatatgaattctatttaatcactttgactagcaaatactaacaacaagacacacacaccaaaaatcaaaaattcactacttttagttaaaaaccacgtaaccacaagaactaatccaatccatgtaatcgggttcttcaaatatttctccaagattttcctcctctaatatgctcaacttaaatgacctattcaataaatctattatgctgctaaatagtttataggacaaataagtatactctaatgacctataaaagatagaaaattaaaaaatcaagtgttcgcttcgctctcactgcccctcgacgttttagtagcctttccctcacttcgttcagtccaagccaactaaaagttttcgggctactctctccttctccccctaataattaattaaaatcttactctgtatatttctgctaatcattcgctaaacagcaaagaaaaaacaaacacgtatcatagatataaatgtaatggcatagtgcgggttttattttcagcctgtatcatagctaaacaaatcgagttgtgtgtccgttttagggcgttctgctagcttgtttaaagtctcttgaatgaatgtatgctctaagtcaaaagaatttgtcagcgcctttatatagctttctttttcttctttttttactttaatgatcgatagcaacaatgatttaacactagcaagttgaatgccaccatttcttcctggtttaatcttaaagaaaatttcctgattcgccttcagtaccttcagcaatttatctaatgtccgttcaggaatgcctagcacttctctaatctcttttttggtcgtcactaaataaggcttgtatacatcgcttttttcgctaatataagccattaaatcttctttccattctgacaaatgaacacgttgacgttcgcttctttttttcttgaatttaaaccacccttgacggacaaataaatctttactggttaaatcacttgatacccaagctttgcaaagaatggtaatgtattccctattagccccttgatagttttctgaataggcacttctaacaattttgattacttctttttcttctaagggttgatctaatcgattattaaactcaaacatattatattcgcacgtttcgattgaatagcctgaactaaagtaggctaaagagagggtaaacatgacgttattacgccctattaaacccttttctcctgaaaatttcgtttcgtgcaataagagattaaaccagggttcatctacttgttttttgccttctgtaccgcttaaaaccgttagacttgaacgagtaaagcccttattatctgtttgtttgaaagaccaatcttgccattctttgaaagaataacggtaattaggatcaaaaaattctacattgtccgttcttggtatgcgagcaataccaaaatgattacacgttagatcaactggcaaagactttccaaaatattctcggatattttgcgaaattattttggctgctttgacagatttaaattctgattttgaagtcacatagactggcgtttctaaaacaaaatatgcttgataacctttatcagatttgataatcatagtaggcataaaacctaaatcaatagcggttgttaaaatatcgcttgctgaaatagtttcttttgccgtgtgaatatcaaaatcaataaagaaggtattgatttgtcttaaattgttttcagaatgtcctttcgtgtatgaacggttttcgtctgcatacgttccataacgataaacgttgggtgtccaatgtgtaaatgtatcttgattttcttgaatcgcttcctcggaagtcagaacaacaccacgaccgccaatcatgcttgattttgagcgatacgcaaaaatagcccctttgcttttacctggcttggtagtgattgagcgaattttactatttttaaatttgtactttaacaagccgtcatgaagcacagtttctacaacaaaagggatattcattcagctgttctcctttcctataaatcctataaaataggttgtttaattaacttggtttgctttttcattcaactgtttcaatattgcatgttttgaaaaagatttttttcctttataagtcaatttttttccactaatcgaataaattattttgttattttctattaacttatatatataatcttccccctccgaagaaaaatacttatctgattttgtttctaagtagatatttctcttttctaactctttcttaaacgtttctagtgtatagatatttgctaattttcttatctccaataaactattttttatataagttttacattcatcatgattcatacaaactccaccttctataaatgaatacaaaaaaagcaatcaaacgatttccgattgattgcttaacaattcttaaattcagtagcttagatacttgaaaactctctgatttccctatataatgatagtacggttatataccgtcttcaaacaaagttaattaaataacttcttacgagggaagagttcatctgactaactgataagcgttggtttggcaatcttatcgggctatgcatttataaaatgtcgtcaaacattttataaatgtgtcatggctcttttttcgtttctattcagttcgttgtttcgttatatctagtataccgcttttaaaaaaaaataagcaacgatttcgtgcattattcacacgaagtcattgcttttttcttcttccatttctaaatccaatgttacttgttctgattctgtttctggttctggttctgttggctcatttgggattaaatccactactagcgttgagttagttaactttgcaatttgttctagtgtttttatggttggatctgattttcctgattctattcgtgaataatttgatctactcatttctaattcttgggagaatttttcttttgatttggtacctctcatcttttttaagcttactag

SEQ ID NO: 26 - DNA, 3223 bp, Saccharomyces cerevisiae
cgtcattataaaaatcattacgaccgagattcccgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaggaaaccgaaatcaaaaaaaagaataaaaaaaaaatgatgaattgaaaagcttgcatgcctgcaggtcgactctagtatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcattttttttttttttttttttttttttttttttttttttttttttttttttttgtacgtcacctggcaaaacgacgatcttcttaggggcagacattacaatggtatatccttgaaatatatataaaaaaaaaaaaaaaaaaaaaaaaaaaaaatgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatatgcatagtaccgagaaactagaggatc

SEQ ID NO: 27 - DNA, 224 bp, Artificial Sequence
  synthesized sequence
gggtgaagtggtcaagacctcactaggcaccttaaaaatagcgcaccctgaagaagatttatttgaggtagcccttgcctacctagcttccaagaaagatatcctaacagcacaagagcggaaagatgttttgttctacatccagaacaacctctgctaaaattcctgaaaaattttgcaaaaagttgttgactttatctacaaggtgtggcataatgtgtgga

SEQ ID NO: 28 - DNA, 96 bp, Artificial Sequence
  synthesized sequence
tggctgcatctatttatattgttttagagctagaaatagcaagttaaaataaggctagtccgttatcaacttgaaaaagtggcaccgagtcggtgc

SEQ ID NO: 29 - DNA, 60 bp, Artificial Sequence
  synthesized sequence
ctagtaaatgcacgagcaacatcttttgttgctcagtgcattttttattttgtacactag

SEQ ID NO: 30 - DNA, 59 bp, Artificial Sequence
  synthesized sequence
gtggatggctgcatctatttatattgttttagagctagaaatagcaagttaaaataagg

SEQ ID NO: 31 - DNA, 55 bp, Artificial Sequence
  synthesized sequence
gccttaagaaatctcattcagcttcctagtgtacaaaataaaaaatgcactgagc

SEQ ID NO: 32 - DNA, 55 bp, Artificial Sequence
  synthesized sequence
gtgcattttttattttgtacactaggaagctgaatgagatttcttaaggctttgc

SEQ ID NO: 33 - DNA, 54 bp, Artificial Sequence
  synthesized sequence
tgtaacataaatatatattttaaaaaaggaggcttatttccgcttttcataagc

SEQ ID NO: 34 - DNA, 59 bp, Artificial Sequence
  synthesized sequence
gaaaagcggaaataagcctccttttttaaaatatatatttatgttacagtaatattgac

SEQ ID NO: 35 - DNA, 55 bp, Artificial Sequence
  synthesized sequence
ggtcgtaatgatttttataatgacgctagtaagcttaaaaaagatgagaggtacc

SEQ ID NO: 36 - DNA, 53 bp, Artificial Sequence
  synthesized sequence
ctctcatcttttttaagcttactagcgtcattataaaaatcattacgaccgag

SEQ ID NO: 37 - DNA, 59 bp, Artificial Sequence
  synthesized sequence
ctagctctaaaacaatataaatagatgcagccatccacacattatgccacaccttgtag

SEQ ID NO: 38 - DNA, 25 bp, Artificial Sequence
  synthesized sequence
catccgctcgttatacaagcgggtg

SEQ ID NO: 39 - DNA, 30 bp, Artificial Sequence
  synthesized sequence
aatccaatatccatcgcaataatggccttg

SEQ ID NO: 40 - DNA, 25 bp, Artificial Sequence
  synthesized sequence
cccctggtacaaagctagcgccacc

SEQ ID NO: 41 - DNA, 28 bp, Artificial Sequence
  synthesized sequence
cacacttgagcaagctgttaatagcgcg

SEQ ID NO: 42 - DNA, 26 bp, Artificial Sequence
  synthesized sequence
cgcggttgttgttttggtcagtagct

SEQ ID NO: 43 - DNA, 94 bp, Bacillus subtilus
gctgataaacagctgacatcaactaaaagtttcattaaatactttgaaaaaagttgttgacttaaaagaagctaaatgttatagtaattgtaca

SEQ ID NO: 44 - DNA, 87 bp, Bacillus subtilus
gtgagaagcaaaaaattgtggatcagcttgttgtttgcgttaacgttaatctttacgatggcgttcagcaacatgtctgcgcaggct

SEQ ID NO: 45 - DNA, 252 bp, Bacillus subtilus
gctgaagaagcaaaagaaaaatatttaattggctttaatgagcaggaagctgtcagtgagtttgtagaacaagtagaggcaaatgacaatgtcgccattctctctgaggaagaggaagtcgaaattgaattgcttcatgaatttgaaacgattcctgttttatccgttgagttaagcccagaagatgtggacgcgcttgaactcgatccagcgatttcttatattgaagaggatgcagaagtaacgacaatg

SEQ ID NO: 46 - DNA, 807 bp, Bacillus clausii
gcgcaatcggtaccatggggaattagccgtgtgcaagccccagctgcccataaccgtggattgacaggttctggtgtaaaagttgctgtcctcgatacaggtatttccactcatccagacttaaatattcgtggtggcgctagctttgtaccaggggaaccatccactcaagatgggaatgggcatggcacgcatgtggctgggacgattgctgctttaaacaattcgattggcgttcttggcgtagcaccgaacgcggaactatacgctgttaaagtattaggggcgagcggttcaggttcggtcagctcgattgcccaaggattggaatgggcagggaacaatgttatgcacgttgctaatttgagtttaggactgcaggcaccaagtgccacacttgagcaagctgttaatagcgcgacttctagaggcgttcttgttgtagcggcatctgggaattcaggtgcaggctcaatcagctatccggcccgttatgcgaacgcaatggcagtcggagctactgaccaaaacaacaaccgcgccagcttttcacagtatggcgcagggcttgacattgtcgcaccaggtgtaaacgtgcagagcacatacccaggttcaacgtatgccagcttaaacggtacatcgatggctactcctcatgttgcaggtgcagcagcccttgttaaacaaaagaacccatcttggtccaatgtacaaatccgcaatcatctaaagaatacggcaacgagcttaggaagcacgaacttgtatggaagcggacttgtcaatgcagaagcggcaacacgt

SEQ ID NO: 47 - DNA, 247 bp, Bacillus subtilus
acataaaaaaccggccttggccccgccggttttttattatttttcttcctccgcatgttcaatccgctccataatcgacggatggctccctctgaaaattttaacgagaaacggcgggttgacccggctcagtcccgtaacggccaagtcctgaaacgtctcaatcgccgcttcccggtttccggtcagctcaatgccgtaacggtcggcggcgttttcctgataccgggagacggcattcgtaatc

SEQ ID NO: 48 - DNA, 39 bp, Artificial Sequence
  synthesized sequence
gaaaattaggggtgagctccgattacgaatgccgtctcc

SEQ ID NO: 49 - DNA, 43 bp, Artificial Sequence
  synthesized sequence
ttcctgcttataggaaggaccgctgataaacagctgacatcaa

SEQ ID NO: 50 - DNA, 39 bp, Artificial Sequence
  synthesized sequence
ggagacggcattcgtaatcggagctcacccctaattttc

SEQ ID NO: 51 - DNA, 43 bp, Artificial Sequence
  synthesized sequence
acgacgttgtaaaacgacgggccgaatatgtagtttgtggatc

SEQ ID NO: 52 - DNA, 43 bp, Artificial Sequence
  synthesized sequence
ttgatgtcagctgtttatcagcggtccttcctataagcaggaa

SEQ ID NO: 53 - DNA, 39 bp, Artificial Sequence
  synthesized sequence
tgaccatgattacgccaagcgcaaattccgggcttcata

SEQ ID NO: 54 - DNA, 39 bp, Artificial Sequence
  synthesized sequence
tatgaagcccggaatttgcgcttggcgtaatcatggtca

SEQ ID NO: 55 - DNA, 43 bp, Artificial Sequence
  synthesized sequence
gatccacaaactacatattcggcccgtcgttttacaacgtcgt

SEQ ID NO: 56 - DNA, 20 bp, Artificial Sequence
  synthesized sequence
agcgtatagttccgcgttcg

SEQ ID NO: 57 - DNA, 23 bp, Artificial Sequence
  synthesized sequence
gctagctttgtaccaggggaacc

SEQ ID NO: 58 - DNA, 20 bp, Artificial Sequence
  synthesized sequence
ggacttgtcaatgcagaagc

SEQ ID NO: 59 - DNA, 8439 bp, Artificial Sequence
  synthesized sequence
gctagtagccctctttaaagggcttttatgtttattttgagaaagatataaaatcaatatatcccttttctccgatttttacaacggcattgtaggactttctatctttcgttttgattccttttaccagggtttcttttccctctagtaattcttttacatttgttttggtgagttttttctttctaaaatgttcagctaaagtaaacttacattcaggataatttgaacaaccataaaacgatttttttaatacaatattgttgccacacttaggacattttcctacaatacttttttctgcttctttttctttctgttcctggtaatcagaaaaatttagtttttctatatcgttaggtacagcttccagtaaatgaacaatgaattttttgatattcgtaataaagttctcttgattgccttctcttttaccgattttttttaaatacgtttcccatttagccgtcatttcagcactcgttaaaaggtgctgactttcaactgcctggcacaataattttcctttttcagttacaacaagcttattcttaatcacttggatatattctttttgtttcaaggcttcaataatgcttgctcttgtcgcttctgttccaatcccctcaacttcttgtaaaatcttgattgcttcttcatcatcaaccgttttattcgccgttttcatagcagttaataatgtaccctctgtaaaagctttcggtggttgtgtttctttttcggcactcttaacgtcaacctcggcatgttctccaatgataaccaagggtaacgtttgaacgtcctcttcttcttctttggtttgttgtttgaaaagaattttccaaccttcttgctttggtgtcttgcctattgattgaaaaagtaagtcggctactttggtttgtatcttagtttcttcatacaaataatcaggtagaaacatggcaacggtcgttttaacgactaaagcatatatttttcgttgtaaatcgtccattttcgctaatgcagattcggtaggtacttgttttgttgggataatggcgtggtgttcctgtaccttactaccgtccacataacgctttctaggctctgtttgaaccatttcaagatcaagtcctaaaaaaccgctatatttgccaaaattcgcttttaaataagcaaattcgttctctgtaataaatggtgtatctgttcttggataactcaataattttgcttcatacagtccttgcatagcttttaaagtttggctcgctgtcgctttataaagctgattaacttttgattgcaaactacttaaagaaaacaaactcggactattcgttttcttctctttggtttgaacatcagcaattatcccctcttgattgcctattttagcttgttcagaagaaacaaaagctaaaagctcctcttgggttttaaagcgctgtgtggggcttataacgcccttaaatgacccttggtttacttttatactagcttccacctcgaaaaaaggttcttttctaaagttttctatggcttcctggcgctgaaaaataagatataaggtgggcgtttgaacacgtcctagtgaaaatgtaccttgtacgcccttctgctgtaaatttaacgtatacaaagggcttgcattcatgccgatcaaccaatcggcaatttggcgtgtttgcgcttcttgataaaagggatagtaattcattcctggttgcaaattttgaaaaccgctacggatcacatctttttctaaactattgatccatagtcttttatacgttttatctttagaaaaggcatttgctttatgaatgatcgaccaggcaatgttttcgccttctctgtcgctatctgttgcgacaatgattgtatttgcttgttttaaaagttcagcaacaattttaaactgcttttttttatctgttgccacttcaaaatcgtatcgatcaggaaaaatcggcaatgattcaagtttccaattttgccacttttcgtcataatgacctggttctgctaactcgactaaatgcccaaaaccaaacgtgataaacgtttcatctgcaaagattgggtctttgatctcaaaataacagtcttttttggtgctttgttttaaagcacttgcataggctaatgcctggcttggtttttcagctaaaataaccgtactcattaactatccctcttttcattgttttttctttgatcgactgtcacgttgtatcttgatcgataccttctaaacgttcggcgattaattccagtttgttcttcaacttctttatcggataaaccgttcaaaaacaaatcgaaagcgtgttgtaaacgtggatcattttctttgaatttcagttgtcgccctttgaattttccttttttcttagcaatttcaattccttgggcttggcgttctttaattcgtttgcgttcagattccgcttggtacttatacaattcaatcactaaattattaatcagccgtcttaaattttcatcttcaataccattcattgagggtaaatttaagacttccagggttgcccccttaatttgaatttgattcatcaattctgttaattctttattatttcgtcctaatcgatctaattcagtaacaacaactatatccccttcacgaatatagttaagcatagcttgtaattgtgggcgttcgaccgattgaccgcttgctttgtctgaaaagaccttagaaacgccctctaacgctttcagttgtctatctaagttctgttctttgctactgacacgtgcataccctactttagtcatgttaaaaaacacctcccttgttaagaaaaaaataaattggatatgaattctatttaatcactttgactagcaaatactaacaacaagacacacacaccaaaaatcaaaaattcactacttttagttaaaaaccacgtaaccacaagaactaatccaatccatgtaatcgggttcttcaaatatttctccaagattttcctcctctaatatgctcaacttaaatgacctattcaataaatctattatgctgctaaatagtttataggacaaataagtatactctaatgacctataaaagatagaaaattaaaaaatcaagtgttcgcttcgctctcactgcccctcgacgttttagtagcctttccctcacttcgttcagtccaagccaactaaaagttttcgggctactctctccttctccccctaataattaattaaaatcttactctgtatatttctgctaatcattcgctaaacagcaaagaaaaaacaaacacgtatcatagatataaatgtaatggcatagtgcgggttttattttcagcctgtatcatagctaaacaaatcgagttgtgtgtccgttttagggcgttctgctagcttgtttaaagtctcttgaatgaatgtatgctctaagtcaaaagaatttgtcagcgcctttatatagctttctttttcttctttttttactttaatgatcgatagcaacaatgatttaacactagcaagttgaatgccaccatttcttcctggtttaatcttaaagaaaatttcctgattcgccttcagtaccttcagcaatttatctaatgtccgttcaggaatgcctagcacttctctaatctcttttttggtcgtcactaaataaggcttgtatacatcgcttttttcgctaatataagccattaaatcttctttccattctgacaaatgaacacgttgacgttcgcttctttttttcttgaatttaaaccacccttgacggacaaataaatctttactggttaaatcacttgatacccaagctttgcaaagaatggtaatgtattccctattagccccttgatagttttctgaataggcacttctaacaattttgattacttctttttcttctaagggttgatctaatcgattattaaactcaaacatattatattcgcacgtttcgattgaatagcctgaactaaagtaggctaaagagagggtaaacatgacgttattacgccctattaaacccttttctcctgaaaatttcgtttcgtgcaataagagattaaaccagggttcatctacttgttttttgccttctgtaccgcttaaaaccgttagacttgaacgagtaaagcccttattatctgtttgtttgaaagaccaatcttgccattctttgaaagaataacggtaattaggatcaaaaaattctacattgtccgttcttggtatgcgagcaataccaaaatgattacacgttagatcaactggcaaagactttccaaaatattctcggatattttgcgaaattattttggctgctttgacagatttaaattctgattttgaagtcacatagactggcgtttctaaaacaaaatatgcttgataacctttatcagatttgataatcatagtaggcataaaacctaaatcaatagcggttgttaaaatatcgcttgctgaaatagtttcttttgccgtgtgaatatcaaaatcaataaagaaggtattgatttgtcttaaattgttttcagaatgtcctttcgtgtatgaacggttttcgtctgcatacgttccataacgataaacgttgggtgtccaatgtgtaaatgtatcttgattttcttgaatcgcttcctcggaagtcagaacaacaccacgaccgccaatcatgcttgattttgagcgatacgcaaaaatagcccctttgcttttacctggcttggtagtgattgagcgaattttactatttttaaatttgtactttaacaagccgtcatgaagcacagtttctacaacaaaagggatattcattcagctgttctcctttcctataaatcctataaaataggttgtttaattaacttggtttgctttttcattcaactgtttcaatattgcatgttttgaaaaagatttttttcctttataagtcaatttttttccactaatcgaataaattattttgttattttctattaacttatatatataatcttccccctccgaagaaaaatacttatctgattttgtttctaagtagatatttctcttttctaactctttcttaaacgtttctagtgtatagatatttgctaattttcttatctccaataaactattttttatataagttttacattcatcatgattcatacaaactccaccttctataaatgaatacaaaaaaagcaatcaaacgatttccgattgattgcttaacaattcttaaattcagtagcttagatacttgaaaactctctgatttccctatataatgatagtacggttatataccgtcttcaaacaaagttaattaaataacttcttacgagggaagagttcatctgactaactgataagcgttggtttggcaatcttatcgggctatgcatttataaaatgtcgtcaaacattttataaatgtgtcatggctcttttttcgtttctattcagttcgttgtttcgttatatctagtataccgcttttaaaaaaaaataagcaacgatttcgtgcattattcacacgaagtcattgcttttttcttcttccatttctaaatccaatgttacttgttctgattctgtttctggttctggttctgttggctcatttgggattaaatccactactagcgttgagttagttaactttgcaatttgttctagtgtttttatggttggatctgattttcctgattctattcgtgaataatttgatctactcatttctaattcttgggagaatttttcttttgatttggtacctctcatcttttttaagcttactagcgggccccggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatccccctcgagcaccaggtgactagtttttaaaatatatatttatgttacagtaatattgacttttaaaaaaggattgattctaatgaagaaagcagacaagtaagcctcctaaattcactttagataaaaatttaggaggcatatcaaatgaactttaataaaattgatttagacaattggaagagaaaagagatatttaatcattatttgaaccaacaaacgacttttagtataaccacagaaattgatattagtgttttataccgaaacataaaacaagaaggatataaattttaccctgcatttattttcttagtgacaagggtgataaactcaaatacagcttttagaactggttacaatagcgacggagagttaggttattgggataagttagagccactttatacaatttttgatggtgtatctaaaacattctctggtatttggactcctgtaaagaatgacttcaaagagttttatgatttatacctttctgatgtagagaaatataatggttcggggaaattgtttcccaaaacacctatacctgaaaatgctttttctctttctattattccatggacttcatttactgggtttaacttaaatatcaataataatagtaattaccttctacccattattacagcaggaaaattcattaataaaggtaattcaatatatttaccgctatctttacaggtacatcattctgtttgtgatggttatcatgcaggattgtttatgaactctattcaggaattgtcagataggcctaatgactggcttttataatatgaggtcgacgcatgc

SEQ ID NO: 60 - DNA, 13575 bp, Artificial Sequence
  synthesized sequence
tcgcttcgatatcgtatggccgaacgatcggctgcctaagtcattaaaaaaagaccgttgacgatgaacggccattttttcttcatgattcatctcccttgaaaatgaacgtttcctttaaaagtctgccttcgcttcctttttccgtccaaatcatgtccacttccaattcggtcgcattctctgccataagcagattgcggtgcttgacagagcttccgtgatttaagcttttggctaaagaaaccggattttcgtgaggaatgttccaaagcgaaaatttcgttgaagaattaggctcgttccgatagacaaaaatttccgccgaagccacatcgtgtccaatgtttttgatttcgacagcataagtatgaaacgctcctttttccggtttcgccattccgtttccggcttcggctttaccgatttgaacttcccattgttttgaagcttgtttaaccggcaattcttcaaacgtcagcgctttcgtttcaaatgacggaacaatcaagccgaacaggcacgatgcacatatgaataaccgcatatttttcactcctggatggtttatttccccatatgttttcctaattcataaatcctatgtataccggcgtttcactccggcttccggcagggtaaataaaaggattggagtgatcaagcaatgccgcaaaacaataacaatcgatatgaactccctgcttcactgattccggcaatattctcaccccatcctgtgttgtaaatgaattgtagcgcgtttcggaggtccaggcaacagtctctcttcttctttgtcttcttgctgtgccgttctccgcctctccacctccggctgtcatataggttatactgacatagatcggggtgaagataaaataaaaaaacggatttccttcaggaaatccgtcctctgttaacctcgagaagcttttaggtcgtttttctaggaacccaaaccgaaaccgaaccgccattgactttgaattccccccatccatcactgttgatggtgacggtgtcactccggttgccggtaaggtcatagaacacttttccagcgtgttgtttgccaacgtacatccatttgcttcctcccggcccatcggtgatcagtgcggccagcccggatcctggtttttcagtgaccccttcccttgtccacccgatgatgtcggagtgatcaagataatcatgttgcgttccgtaagcataatccctgcgcgcgatgaggagcggatcgattttgcttttcagcgaaggaatgttatattgtggaatgccataatagtcaccataaaagacgcacgggtatccttcctgccgagttagaataaaggcgtaagccaacggtttgaaccatgggtcgacccatgactgaagcgcttggccgggttcggtgtcatgattatcaacgaaggtgacggccaatgtcggttgatctttcatgagagtattggtcattaacgtgcgcatatcaaatgcgccccctgatttggaagcggtataaaatttgttgtgtaacggggcatcaaacaaagacatcgttccgtttgttttcgtaatgtaattgtgcaacttgttgatgtcatagctccaatattccccgacggtaaatagcggcttgccagtctgagaacgcacatacgacaaccaatcaggaaaaaaactgaacttaatatgcttgacggcatcaagccggaacccatcaatgttcgttgtgttgacataccatttcccccagtttttcagctcggtcacgacttcgggatgatccatatcaaggtcggcatacattaagtagtcatagtttccgttttctgtgtctaccggccaatcccacgctttgccgatgcccctgaatttgtaaatgcggcttaattttcggctttcgtcccaatcaacgccgtcaaaatggtaccagcgccacttaaagctggagtaggtgttgccccgcccgggaaaatcaaatttcgtccatgcttggatttgataggtgcccgagatttcttggttgcggtcggacggattgacttcgacggcgtccacccattccgtgccgtcagcgccgcctttatggtcgaacacgacatcggcgtacacttgcattccagcggcgtgggcggcttgaatggcttgaagatattgagcttttgttccatattttgtgcggacggtccctttttgattgaattcgccgaggtcatacaagtcgtatactccgtaccctacgtcgctgcggcttgttcctttgtaagcgggcggcagccaaagagcggtgatgccaaggctggataagttgttggcttcattggccactttggtccataacgtgccatcatccggcaagtaccattcaaaatactgcatcatggtaccgttaaacggtgcggctgcgctagctgcagaatgaggcagcaagaagatgagcgcaaataacagcgtcagcaatcgggcgtaaagccgtttttgttgtttcattctttaccctctccttttaaaaaaattcagagtagacttacttaaaagactattctgttttattactataacatttagcttcttttaagtcaacaactttttaaaatattttttgaaaaaataggatattgatgtcagctgtttatcagcgacctcgaggggggttaagagacccgctaagaagtacatataaagcgtttcggaggtccaggcaacagtctctcttcttctttgtcttcttgctgtgccgttctccgcctctccacctccggctgtcatataggttatactgacatagatcggggtgaagatatgaacagcaaatcacgattgacgcctgaaatgtggcgggcgattacggaaaacgattccgcctatgacggagttttttattacgcggtcaaaacgaccggcatattttgccgcccttcctgcaaatcgagagttccgcaaatcgacaatgtgcagatctttttcaatgcaaaagatgctttatcagaagggtaccgcccctgcaaacgctgcaatccggccggggcgctgctgccggatgaagagctggcacagcgtgtggtggaaatcatcgaggaatcttatcgcgatccgctgtctctgcaagctttggctgacaggtgccatatcagcccttttcacctgcagcggacatttaaacgaatcaaaggcgtctcgccggcagaatacatcctgcagaaaaggattgagaaagcggtcgatttgcttgctcattcagaacgatcgatcgcagaaatcgcttcaatggtaggcattgcgaatgcggaatattttgcctctttgtttaaaaagaagactgggcaatcgccgacgtcgtaccggcaaatgaaacggaggtaaacatatggacgatcaaatcgtttttaaaatatatatttatgttacagtaatattgacttttaaaaaaggattgattctaatgaagaaagcagacaagtaagcctcctaaattcactttagataaaaatttaggaggcatatcaaatgaactttaataaaattgatttagacaattggaagagaaaagagatatttaatcattatttgaaccaacaaacgacttttagtataaccacagaaattgatattagtgttttataccgaaacataaaacaagaaggatataaattttaccctgcatttattttcttagtgacaagggtgataaactcaaatacagcttttagaactggttacaatagcgacggagagttaggttattgggataagttagagccactttatacaatttttgatggtgtatctaaaacattctctggtatttggactcctgtaaagaatgacttcaaagagttttatgatttatacctttctgatgtagagaaatataatggttcggggaaattgtttcccaaaacacctatacctgaaaatgctttttctctttctattattccatggacttcatttactgggtttaacttaaatatcaataataatagtaattaccttctacccattattacagcaggaaaattcattaataaaggtaattcaatatatttaccgctatctttacaggtacatcattctgtttgtgatggttatcatgcaggattgtttatgaactctattcaggaattgtcagataggcctaatgactggcttttataatatgaggtcgacgcatgcgctagtagccctctttaaagggcttttatgtttattttgagaaagatataaaatcaatatatcccttttctccgatttttacaacggcattgtaggactttctatctttcgttttgattccttttaccagggtttcttttccctctagtaattcttttacatttgttttggtgagttttttctttctaaaatgttcagctaaagtaaacttacattcaggataatttgaacaaccataaaacgatttttttaatacaatattgttgccacacttaggacattttcctacaatacttttttctgcttctttttctttctgttcctggtaatcagaaaaatttagtttttctatatcgttaggtacagcttccagtaaatgaacaatgaattttttgatattcgtaataaagttctcttgattgccttctcttttaccgattttttttaaatacgtttcccatttagccgtcatttcagcactcgttaaaaggtgctgactttcaactgcctggcacaataattttcctttttcagttacaacaagcttattcttaatcacttggatatattctttttgtttcaaggcttcaataatgcttgctcttgtcgcttctgttccaatcccctcaacttcttgtaaaatcttgattgcttcttcatcatcaaccgttttattcgccgttttcatagcagttaataatgtaccctctgtaaaagctttcggtggttgtgtttctttttcggcactcttaacgtcaacctcggcatgttctccaatgataaccaagggtaacgtttgaacgtcctcttcttcttctttggtttgttgtttgaaaagaattttccaaccttcttgctttggtgtcttgcctattgattgaaaaagtaagtcggctactttggtttgtatcttagtttcttcatacaaataatcaggtagaaacatggcaacggtcgttttaacgactaaagcatatatttttcgttgtaaatcgtccattttcgctaatgcagattcggtaggtacttgttttgttgggataatggcgtggtgttcctgtaccttactaccgtccacataacgctttctaggctctgtttgaaccatttcaagatcaagtcctaaaaaaccgctatatttgccaaaattcgcttttaaataagcaaattcgttctctgtaataaatggtgtatctgttcttggataactcaataattttgcttcatacagtccttgcatagcttttaaagtttggctcgctgtcgctttataaagctgattaacttttgattgcaaactacttaaagaaaacaaactcggactattcgttttcttctctttggtttgaacatcagcaattatcccctcttgattgcctattttagcttgttcagaagaaacaaaagctaaaagctcctcttgggttttaaagcgctgtgtggggcttataacgcccttaaatgacccttggtttacttttatactagcttccacctcgaaaaaaggttcttttctaaagttttctatggcttcctggcgctgaaaaataagatataaggtgggcgtttgaacacgtcctagtgaaaatgtaccttgtacgcccttctgctgtaaatttaacgtatacaaagggcttgcattcatgccgatcaaccaatcggcaatttggcgtgtttgcgcttcttgataaaagggatagtaattcattcctggttgcaaattttgaaaaccgctacggatcacatctttttctaaactattgatccatagtcttttatacgttttatctttagaaaaggcatttgctttatgaatgatcgaccaggcaatgttttcgccttctctgtcgctatctgttgcgacaatgattgtatttgcttgttttaaaagttcagcaacaattttaaactgcttttttttatctgttgccacttcaaaatcgtatcgatcaggaaaaatcggcaatgattcaagtttccaattttgccacttttcgtcataatgacctggttctgctaactcgactaaatgcccaaaaccaaacgtgataaacgtttcatctgcaaagattgggtctttgatctcaaaataacagtcttttttggtgctttgttttaaagcacttgcataggctaatgcctggcttggtttttcagctaaaataaccgtactcattaactatccctcttttcattgttttttctttgatcgactgtcacgttgtatcttgatcgataccttctaaacgttcggcgattaattccagtttgttcttcaacttctttatcggataaaccgttcaaaaacaaatcgaaagcgtgttgtaaacgtggatcattttctttgaatttcagttgtcgccctttgaattttccttttttcttagcaatttcaattccttgggcttggcgttctttaattcgtttgcgttcagattccgcttggtacttatacaattcaatcactaaattattaatcagccgtcttaaattttcatcttcaataccattcattgagggtaaatttaagacttccagggttgcccccttaatttgaatttgattcatcaattctgttaattctttattatttcgtcctaatcgatctaattcagtaacaacaactatatccccttcacgaatatagttaagcatagcttgtaattgtgggcgttcgaccgattgaccgcttgctttgtctgaaaagaccttagaaacgccctctaacgctttcagttgtctatctaagttctgttctttgctactgacacgtgcataccctactttagtcatgttaaaaaacacctcccttgttaagaaaaaaataaattggatatgaattctatttaatcactttgactagcaaatactaacaacaagacacacacaccaaaaatcaaaaattcactacttttagttaaaaaccacgtaaccacaagaactaatccaatccatgtaatcgggttcttcaaatatttctccaagattttcctcctctaatatgctcaacttaaatgacctattcaataaatctattatgctgctaaatagtttataggacaaataagtatactctaatgacctataaaagatagaaaattaaaaaatcaagtgttcgcttcgctctcactgcccctcgacgttttagtagcctttccctcacttcgttcagtccaagccaactaaaagttttcgggctactctctccttctccccctaataattaattaaaatcttactctgtatatttctgctaatcattcgctaaacagcaaagaaaaaacaaacacgtatcatagatataaatgtaatggcatagtgcgggttttattttcagcctgtatcatagctaaacaaatcgagttgtgtgtccgttttagggcgttctgctagcttgtttaaagtctcttgaatgaatgtatgctctaagtcaaaagaatttgtcagcgcctttatatagctttctttttcttctttttttactttaatgatcgatagcaacaatgatttaacactagcaagttgaatgccaccatttcttcctggtttaatcttaaagaaaatttcctgattcgccttcagtaccttcagcaatttatctaatgtccgttcaggaatgcctagcacttctctaatctcttttttggtcgtcactaaataaggcttgtatacatcgcttttttcgctaatataagccattaaatcttctttccattctgacaaatgaacacgttgacgttcgcttctttttttcttgaatttaaaccacccttgacggacaaataaatctttactggttaaatcacttgatacccaagctttgcaaagaatggtaatgtattccctattagccccttgatagttttctgaataggcacttctaacaattttgattacttctttttcttctaagggttgatctaatcgattattaaactcaaacatattatattcgcacgtttcgattgaatagcctgaactaaagtaggctaaagagagggtaaacatgacgttattacgccctattaaacccttttctcctgaaaatttcgtttcgtgcaataagagattaaaccagggttcatctacttgttttttgccttctgtaccgcttaaaaccgttagacttgaacgagtaaagcccttattatctgtttgtttgaaagaccaatcttgccattctttgaaagaataacggtaattaggatcaaaaaattctacattgtccgttcttggtatgcgagcaataccaaaatgattacacgttagatcaactggcaaagactttccaaaatattctcggatattttgcgaaattattttggctgctttgacagatttaaattctgattttgaagtcacatagactggcgtttctaaaacaaaatatgcttgataacctttatcagatttgataatcatagtaggcataaaacctaaatcaatagcggttgttaaaatatcgcttgctgaaatagtttcttttgccgtgtgaatatcaaaatcaataaagaaggtattgatttgtcttaaattgttttcagaatgtcctttcgtgtatgaacggttttcgtctgcatacgttccataacgataaacgttgggtgtccaatgtgtaaatgtatcttgattttcttgaatcgcttcctcggaagtcagaacaacaccacgaccgccaatcatgcttgattttgagcgatacgcaaaaatagcccctttgcttttacctggcttggtagtgattgagcgaattttactatttttaaatttgtactttaacaagccgtcatgaagcacagtttctacaacaaaagggatattcattcagctgttctcctttcctataaatcctataaaataggttgtttaattaacttggtttgctttttcattcaactgtttcaatattgcatgttttgaaaaagatttttttcctttataagtcaatttttttccactaatcgaataaattattttgttattttctattaacttatatatataatcttccccctccgaagaaaaatacttatctgattttgtttctaagtagatatttctcttttctaactctttcttaaacgtttctagtgtatagatatttgctaattttcttatctccaataaactattttttatataagttttacattcatcatgattcatacaaactccaccttctataaatgaatacaaaaaaagcaatcaaacgatttccgattgattgcttaacaattcttaaattcagtagcttagatacttgaaaactctctgatttccctatataatgatagtacggttatataccgtcttcaaacaaagttaattaaataacttcttacgagggaagagttcatctgactaactgataagcgttggtttggcaatcttatcgggctatgcatttataaaatgtcgtcaaacattttataaatgtgtcatggctcttttttcgtttctattcagttcgttgtttcgttatatctagtataccgcttttaaaaaaaaataagcaacgatttcgtgcattattcacacgaagtcattgcttttttcttcttccatttctaaatccaatgttacttgttctgattctgtttctggttctggttctgttggctcatttgggattaaatccactactagcgttgagttagttaactttgcaatttgttctagtgtttttatggttggatctgattttcctgattctattcgtgaataatttgatctactcatttctaattcttgggagaatttttcttttgatttggtacctctcatcttttttaagcttactagcgtcattataaaaatcattacgaccgagattcccgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaggaaaccgaaatcaaaaaaaagaataaaaaaaaaatgatgaattgaaaagcttgcatgcctgcaggtcgactctagtatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcattttttttttttttttttttttttttttttttttttttttttttttttttttgtacgtcacctggcaaaacgacgatcttcttaggggcagacattacaatggtatatccttgaaatatatataaaaaaaaaaaaaaaaaaaaaaaaaaaaaatgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatatgcatagtaccgagaaactagaggatcgggtgaagtggtcaagacctcactaggcaccttaaaaatagcgcaccctgaagaagatttatttgaggtagcccttgcctacctagcttccaagaaagatatcctaacagcacaagagcggaaagatgttttgttctacatccagaacaacctctgctaaaattcctgaaaaattttgcaaaaagttgttgactttatctacaaggtgtggcataatgtgtggaatcccgcatcagattcacttgttttagagctagaaatagcaagttaaaataaggctagtccgttatcaacttgaaaaagtggcaccgagtcggtgcctagtaaatgcacgagcaacatcttttgttgctcagtgcattttttattttgtacactag

SEQ ID NO: 61 - DNA, 8347 bp, Artificial Sequence
  synthesized sequence
gaattcctccattttcttctgctatcaaaataacagactcgtgattttccaaacgagctttcaaaaaagcctctgccccttgcaaatcggatgcctgtctataaaattcccgatattggttaaacagcggcgcaatggcggccgcatctgatgtctttgcttggcgaatgttcatcttatttcttcctccctctcaataattttttcattctatcccttttctgtaaagtttatttttcagaatacttttatcatcatgctttgaaaaaatatcacgataatatccattgttctcacggaagcacacgcaggtcatttgaacgaattttttcgacaggaatttgccgggactcaggagcatttaacctaaaaaagcatgacatttcagcataatgaacatttactcatgtctattttcgttcttttctgtatgaaaatagttatttcgagtctctacggaaatagcgagagatgatatacctaaatagagataaaatcatctcaaaaaaatgggtctactaaaatattattccatctattacaataaattcacagaatagtcttttaagtaagtctactctgaatttttttaaaaggagagggtaactagtggccccaaaaaagaaacgcaaggttatggataaaaaatacagcattggtctggatatcggaaccaacagcgttgggtgggcagtaataacagatgaatacaaagtgccgtcaaaaaaatttaaggttctggggaatacagatcgccacagcataaaaaagaatctgattggggcattgctgtttgattcgggtgagacagctgaggccacgcgtctgaaacgtacagcaagaagacgttacacacgtcgtaaaaatcgtatttgctacttacaggaaattttttctaacgaaatggccaaggtagatgatagtttcttccatcgtctcgaagaatcttttctggttgaggaagataaaaaacacgaacgtcaccctatctttggcaatatcgtggatgaagtggcctatcatgaaaaataccctacgatttatcatcttcgcaagaagttggttgatagtacggacaaagcggatctgcgtttaatctatcttgcgttagcgcacatgatcaaatttcgtggtcatttcttaattgaaggtgatctgaatcctgataactctgatgtggacaaattgtttatacaattagtgcaaacctataatcagctgttcgaggaaaaccccattaatgcctctggagttgatgccaaagcgattttaagcgcgagactttctaagtcccggcgtctggagaatctgatcgcccagttaccaggggaaaagaaaaatggtctgtttggtaatctgattgccctcagtctggggcttaccccgaacttcaaatccaattttgacctggctgaggacgcaaagctgcagctgagcaaagatacttatgatgatgacctcgacaatctgctcgcccagattggtgaccaatatgcggatctgtttctggcagcgaagaatctttcggatgctatcttgctgtcggatattctgcgtgttaataccgaaatcaccaaagcgcctctgtctgcaagtatgatcaagagatacgacgagcaccaccaggacctgactcttcttaaggcactggtacgccaacagcttccggagaaatacaaagaaatattcttcgaccagtccaagaatggttacgcgggctacatcgatggtggtgcatcacaggaagagttctataaatttattaaaccaatccttgagaaaatggatggcacggaagagttacttgttaaacttaaccgcgaagacttgcttagaaagcaacgtacattcgacaacggctccatcccacaccagattcatttaggtgaacttcacgccatcttgcgcagacaagaagatttctatcccttcttaaaagacaatcgggagaaaatcgagaagatcctgacgttccgcattccctattatgtcggtcccctggcacgtggtaattctcggtttgcctggatgacgcgcaaaagtgaggaaaccatcaccccttggaactttgaagaagtcgtggataaaggtgctagcgcgcagtcttttatagaaagaatgacgaacttcgataaaaacttgcccaacgaaaaagtcctgcccaagcactctcttttatatgagtactttactgtgtacaacgaactgactaaagtgaaatacgttacggaaggtatgcgcaaacctgcctttcttagtggcgagcagaaaaaagcaattgtcgatcttctctttaaaacgaatcgcaaggtaactgtaaaacagctgaaggaagattatttcaaaaagatcgaatgctttgattctgtcgagatctcgggtgtcgaagatcgtttcaacgcttccttagggacctatcatgatttgctgaagataataaaagacaaagactttctcgacaatgaagaaaatgaagatattctggaggatattgttttgaccttgaccttattcgaagatagagagatgatcgaggagcgcttaaaaacctatgcccacctgtttgatgacaaagtcatgaagcaattaaagcgccgcagatatacggggtggggccgcttgagccgcaagttgattaacggtattagagacaagcagagcggaaaaactatcctggatttcctcaaatctgacggatttgcgaaccgcaattttatgcagcttatacatgatgattcgcttacattcaaagaggatattcagaaggctcaggtgtctgggcaaggtgattcactccacgaacatatagcaaatttggccggctctcctgcgattaagaaggggatcctgcaaacagttaaagttgtggatgaacttgtaaaagtaatgggccgccacaagccggagaatatcgtgatagaaatggcgcgcgagaatcaaacgacacaaaaaggtcaaaagaactcaagagagagaatgaagcgcattgaggaggggataaaggaacttggatctcaaattctgaaagaacatccagttgaaaacactcagctgcaaaatgaaaaattgtacctgtactacctgcagaatggaagagacatgtacgtggatcaggaattggatatcaatagactctcggactatgacgtagatcacattgtccctcagagcttcctcaaggatgattctatagataataaagtacttacgagatcggacaaaaatcgcggtaaatcggataacgtcccatcggaggaagtcgttaaaaagatgaaaaactattggcgtcaactgctgaacgccaagctgatcacacagcgtaagtttgataatctgactaaagccgaacgcggtggtcttagtgaactcgataaagcaggatttataaaacggcagttagtagaaacgcgccaaattacgaaacacgtggctcagatcctcgattctagaatgaatacaaagtacgatgaaaacgataaactgatccgtgaagtaaaagtcattaccttaaaatctaaacttgtgtccgatttccgcaaagattttcagttttacaaggtccgggaaatcaataactatcaccatgcacatgatgcatatttaaatgcggttgtaggcacggcccttattaagaaataccctaaactcgaaagtgagtttgtttatggggattataaagtgtatgacgttcgcaaaatgatcgcgaaatcagaacaggaaatcggtaaggctaccgctaaatactttttttattccaacattatgaatttttttaagaccgaaataactctcgcgaatggtgaaatccgtaaacggcctcttatagaaaccaatggtgaaacgggagaaatcgtttgggataaaggtcgtgactttgccaccgttcgtaaagtcctctcaatgccgcaagttaacattgtcaagaagacggaagttcaaacagggggattctccaaagaatctatcctgccgaagcgtaacagtgataaacttattgccagaaaaaaagattgggatccaaaaaaatacggaggctttgattcccctaccgtcgcgtatagtgtgctggtggttgctaaagtcgagaaagggaaaagcaagaaattgaaatcagttaaagaactgctgggtattacaattatggaaagatcgtcctttgagaaaaatccgatcgactttttagaggccaaggggtataaggaagtgaaaaaagatctcatcatcaaattaccgaagtatagtctttttgagctggaaaacggcagaaaaagaatgctggcctccgcgggcgagttacagaagggaaatgagctggcgctgccttccaaatatgttaattttctgtaccttgccagtcattatgagaaactgaagggcagccccgaagataacgaacagaaacaattattcgtggaacagcataagcactatttagatgaaattatagagcaaattagtgaattttctaagcgcgttatcctcgcggatgctaatttagacaaagtactgtcagcttataataaacatcgggataagccgattagagaacaggccgaaaatatcattcatttgtttaccttaaccaaccttggagcaccagctgccttcaaatatttcgataccacaattgatcgtaaacggtatacaagtacaaaagaagtcttggacgcaaccctcattcatcaatctattactggattatatgagacacgcattgatctttcacagctgggcggagacaagaagaaaaaactgaaactgcaccatcatcaccatcatcatcaccatcattgataactcgagaaagcttacataaaaaaccggccttggccccgccggttttttattatttttcttcctccgcatgttcaatccgctccataatcgacggatggctccctctgaaaattttaacgagaaacggcgggttgacccggctcagtcccgtaacggccaagtcctgaaacgtctcaatcgccgcttcccggtttccggtcagctcaatgccgtaacggtcggcggcgttttcctgataccgggagacggcattcgtaatcgaattcgcggccgcacgcgtccatggggatccccgcgggtcgacctcgagagttacgctagggataacagggtaatataggagctccagtcggcttaaaccagttttcgctggtgcgaaaaaagagtgtcttgtgacacctaaattcaaaatctatcggtcagatttataccgatttgattttatatattcttgaataacatacgccgagttatcacataaaagcgggaaccaatcataaaatttaaacttcattgcataatccattaaactcttaaattctacgattccttgttcatcaataaactcaatcatttctttaattaatttatatctatctgttgttgttttctttaataattcattaacatctacaccgccataaactatcatatcttctttttgatatttaaatttattaggatcgtccatgtgaagcatatatctcacaagacctttcacacttcctgcaatctgcggaatagtcgcattcaattcttctgttaattatttttatctgttcataagatttattaccctcatacatcactagaatatgataatgctcttttttcatcctaccttctgtatcagtatccctatcatgtaatggagacactacaaattgaatgtgtaactcttttaaatactctaaccactcggcttttgctgattctggatataaaacaaatgtccaattacgtcctcttgaatttttcttgttttcagtttcttttattacattttcgctcatgatataataacggtgctaatacacttaacaaaatttagtcatagataggcagcatgccagtgctgtctatctttttttgtttaaaatgcaccgtattcctcctttgcatatttttttattagaataccggttgcatctgatttgctaatattatatttttctttgattctatttaatatctcattttcttctgttgtaagtcttaaagtaacagcaacttttttctcttcttttctatctacaactatcactgtacctcccaacatctgtttttttcactttaacataaaaaacaaccttttaacattaaaaacccaatatttatttatttgtttggacaatggacactggacacctaggggggaggtcgtagtacccccctatgttttctcccctaaataaccccaaaaatctaagaaaaaaagacctcaaaaaggtctttaattaacatctcaaatttcgcatttattccaatttcctttttgcgtgtgatgcgagctcatcggctccgtcgatactatgttatacgccaactttcaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtgactgcagtgagatctggtaatgactctctagcttgaggcatcaaataaaacgaaaggctcagtcgaaagactgggcctttcgttttatctgttgtttgtcggtgaacgctctcctgagtaggacaaatccgccgctctagctaagcagaaggccatcctgacggatggcctttttgcgtttctacaaactcttgttaactctagagctgcctgccgcgtttcggtgatgaagatcttcccgatgattaattaattcagaacgctcggttgccgccgggcgttttttatgaagcttcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgaca

SEQ ID NO: 62 - DNA, 9177 bp, Artificial Sequence
  synthesized sequence
gaattcctccattttcttctgctatcaaaataacagactcgtgattttccaaacgagctttcaaaaaagcctctgccccttgcaaatcggatgcctgtctataaaattcccgatattggttaaacagcggcgcaatggcggccgcatctgatgtctttgcttggcgaatgttcatcttatttcttcctccctctcaataattttttcattctatcccttttctgtaaagtttatttttcagaatacttttatcatcatgctttgaaaaaatatcacgataatatccattgttctcacggaagcacacgcaggtcatttgaacgaattttttcgacaggaatttgccgggactcaggagcatttaacctaaaaaagcatgacatttcagcataatgaacatttactcatgtctattttcgttcttttctgtatgaaaatagttatttcgagtctctacggaaatagcgagagatgatatacctaaatagagataaaatcatctcaaaaaaatgggtctactaaaatattattccatctattacaataaattcacagaatagtcttttaagtaagtctactctgaatttttttaaaaggagagggtaactagtggccccaaaaaagaaacgcaaggttatggataaaaaatacagcattggtctggatatcggaaccaacagcgttgggtgggcagtaataacagatgaatacaaagtgccgtcaaaaaaatttaaggttctggggaatacagatcgccacagcataaaaaagaatctgattggggcattgctgtttgattcgggtgagacagctgaggccacgcgtctgaaacgtacagcaagaagacgttacacacgtcgtaaaaatcgtatttgctacttacaggaaattttttctaacgaaatggccaaggtagatgatagtttcttccatcgtctcgaagaatcttttctggttgaggaagataaaaaacacgaacgtcaccctatctttggcaatatcgtggatgaagtggcctatcatgaaaaataccctacgatttatcatcttcgcaagaagttggttgatagtacggacaaagcggatctgcgtttaatctatcttgcgttagcgcacatgatcaaatttcgtggtcatttcttaattgaaggtgatctgaatcctgataactctgatgtggacaaattgtttatacaattagtgcaaacctataatcagctgttcgaggaaaaccccattaatgcctctggagttgatgccaaagcgattttaagcgcgagactttctaagtcccggcgtctggagaatctgatcgcccagttaccaggggaaaagaaaaatggtctgtttggtaatctgattgccctcagtctggggcttaccccgaacttcaaatccaattttgacctggctgaggacgcaaagctgcagctgagcaaagatacttatgatgatgacctcgacaatctgctcgcccagattggtgaccaatatgcggatctgtttctggcagcgaagaatctttcggatgctatcttgctgtcggatattctgcgtgttaataccgaaatcaccaaagcgcctctgtctgcaagtatgatcaagagatacgacgagcaccaccaggacctgactcttcttaaggcactggtacgccaacagcttccggagaaatacaaagaaatattcttcgaccagtccaagaatggttacgcgggctacatcgatggtggtgcatcacaggaagagttctataaatttattaaaccaatccttgagaaaatggatggcacggaagagttacttgttaaacttaaccgcgaagacttgcttagaaagcaacgtacattcgacaacggctccatcccacaccagattcatttaggtgaacttcacgccatcttgcgcagacaagaagatttctatcccttcttaaaagacaatcgggagaaaatcgagaagatcctgacgttccgcattccctattatgtcggtcccctggcacgtggtaattctcggtttgcctggatgacgcgcaaaagtgaggaaaccatcaccccttggaactttgaagaagtcgtggataaaggtgctagcgcgcagtcttttatagaaagaatgacgaacttcgataaaaacttgcccaacgaaaaagtcctgcccaagcactctcttttatatgagtactttactgtgtacaacgaactgactaaagtgaaatacgttacggaaggtatgcgcaaacctgcctttcttagtggcgagcagaaaaaagcaattgtcgatcttctctttaaaacgaatcgcaaggtaactgtaaaacagctgaaggaagattatttcaaaaagatcgaatgctttgattctgtcgagatctcgggtgtcgaagatcgtttcaacgcttccttagggacctatcatgatttgctgaagataataaaagacaaagactttctcgacaatgaagaaaatgaagatattctggaggatattgttttgaccttgaccttattcgaagatagagagatgatcgaggagcgcttaaaaacctatgcccacctgtttgatgacaaagtcatgaagcaattaaagcgccgcagatatacggggtggggccgcttgagccgcaagttgattaacggtattagagacaagcagagcggaaaaactatcctggatttcctcaaatctgacggatttgcgaaccgcaattttatgcagcttatacatgatgattcgcttacattcaaagaggatattcagaaggctcaggtgtctgggcaaggtgattcactccacgaacatatagcaaatttggccggctctcctgcgattaagaaggggatcctgcaaacagttaaagttgtggatgaacttgtaaaagtaatgggccgccacaagccggagaatatcgtgatagaaatggcgcgcgagaatcaaacgacacaaaaaggtcaaaagaactcaagagagagaatgaagcgcattgaggaggggataaaggaacttggatctcaaattctgaaagaacatccagttgaaaacactcagctgcaaaatgaaaaattgtacctgtactacctgcagaatggaagagacatgtacgtggatcaggaattggatatcaatagactctcggactatgacgtagatcacattgtccctcagagcttcctcaaggatgattctatagataataaagtacttacgagatcggacaaaaatcgcggtaaatcggataacgtcccatcggaggaagtcgttaaaaagatgaaaaactattggcgtcaactgctgaacgccaagctgatcacacagcgtaagtttgataatctgactaaagccgaacgcggtggtcttagtgaactcgataaagcaggatttataaaacggcagttagtagaaacgcgccaaattacgaaacacgtggctcagatcctcgattctagaatgaatacaaagtacgatgaaaacgataaactgatccgtgaagtaaaagtcattaccttaaaatctaaacttgtgtccgatttccgcaaagattttcagttttacaaggtccgggaaatcaataactatcaccatgcacatgatgcatatttaaatgcggttgtaggcacggcccttattaagaaataccctaaactcgaaagtgagtttgtttatggggattataaagtgtatgacgttcgcaaaatgatcgcgaaatcagaacaggaaatcggtaaggctaccgctaaatactttttttattccaacattatgaatttttttaagaccgaaataactctcgcgaatggtgaaatccgtaaacggcctcttatagaaaccaatggtgaaacgggagaaatcgtttgggataaaggtcgtgactttgccaccgttcgtaaagtcctctcaatgccgcaagttaacattgtcaagaagacggaagttcaaacagggggattctccaaagaatctatcctgccgaagcgtaacagtgataaacttattgccagaaaaaaagattgggatccaaaaaaatacggaggctttgattcccctaccgtcgcgtatagtgtgctggtggttgctaaagtcgagaaagggaaaagcaagaaattgaaatcagttaaagaactgctgggtattacaattatggaaagatcgtcctttgagaaaaatccgatcgactttttagaggccaaggggtataaggaagtgaaaaaagatctcatcatcaaattaccgaagtatagtctttttgagctggaaaacggcagaaaaagaatgctggcctccgcgggcgagttacagaagggaaatgagctggcgctgccttccaaatatgttaattttctgtaccttgccagtcattatgagaaactgaagggcagccccgaagataacgaacagaaacaattattcgtggaacagcataagcactatttagatgaaattatagagcaaattagtgaattttctaagcgcgttatcctcgcggatgctaatttagacaaagtactgtcagcttataataaacatcgggataagccgattagagaacaggccgaaaatatcattcatttgtttaccttaaccaaccttggagcaccagctgccttcaaatatttcgataccacaattgatcgtaaacggtatacaagtacaaaagaagtcttggacgcaaccctcattcatcaatctattactggattatatgagacacgcattgatctttcacagctgggcggagacaagaagaaaaaactgaaactgcaccatcatcaccatcatcatcaccatcattgataactcgagaaagcttacataaaaaaccggccttggccccgccggttttttattatttttcttcctccgcatgttcaatccgctccataatcgacggatggctccctctgaaaattttaacgagaaacggcgggttgacccggctcagtcccgtaacggccaagtcctgaaacgtctcaatcgccgcttcccggtttccggtcagctcaatgccgtaacggtcggcggcgttttcctgataccgggagacggcattcgtaatcgaattcgcggccgcacatggccggaaaaaatgtaatcacgatcaaaaggacaaagtcttcgggctttgtcctttttttatgagaaaaacgtgtgatgtaattcacaatcctgtttggctagtttttgtatgataagactgcaggtgatggcgggatcgttgtatatttcttgacaccttttcggcatcgccctaaattcggcgtcctcatattgtgtgaggacgttttattacgtgtttacgaagcaaaagctaaaaccaggagctatttaatggcaacagttaaccagctggtacgcaaaccacgtgctcgcaaagttgcgaaaagcaacgtgcctgcgctggaagcatgcccgcaaaaacgtggcgtatgtactcgtgtatatactaccactcctaaaaaaccgaactccgcgctgcgtaaagtatgccgtgttcgtctgactaacggtttcgaagtgacttcctacatcggtggtgaaggtcacaacctgcaggagcactccgtgatcctgatccgtggcggtcgtgttaaagacctcccgggtgttcgttaccacaccgtacgtggtgcgcttgactgctccggcgttaaagaccgtaagcaggctcgttccaagtatggcgtgaagcgtcctaaggcttaggttaataacaggcctgctggtaatcgcaggcctttttatttttacacctgcgttttagagctagaaatagcaagttaaaataaggctagtccgttatcaacttgaaaaagtggcaccgagtcggtgcgactcctgttgatagatccagtaatgacctcagaactccatctggatttgttcagaacgctcggttgccgccgggcgttttttattggtgagaatgtcgacctcgagagttacgctagggataacagggtaatataggagctccagtcggcttaaaccagttttcgctggtgcgaaaaaagagtgtcttgtgacacctaaattcaaaatctatcggtcagatttataccgatttgattttatatattcttgaataacatacgccgagttatcacataaaagcgggaaccaatcataaaatttaaacttcattgcataatccattaaactcttaaattctacgattccttgttcatcaataaactcaatcatttctttaattaatttatatctatctgttgttgttttctttaataattcattaacatctacaccgccataaactatcatatcttctttttgatatttaaatttattaggatcgtccatgtgaagcatatatctcacaagacctttcacacttcctgcaatctgcggaatagtcgcattcaattcttctgttaattatttttatctgttcataagatttattaccctcatacatcactagaatatgataatgctcttttttcatcctaccttctgtatcagtatccctatcatgtaatggagacactacaaattgaatgtgtaactcttttaaatactctaaccactcggcttttgctgattctggatataaaacaaatgtccaattacgtcctcttgaatttttcttgttttcagtttcttttattacattttcgctcatgatataataacggtgctaatacacttaacaaaatttagtcatagataggcagcatgccagtgctgtctatctttttttgtttaaaatgcaccgtattcctcctttgcatatttttttattagaataccggttgcatctgatttgctaatattatatttttctttgattctatttaatatctcattttcttctgttgtaagtcttaaagtaacagcaacttttttctcttcttttctatctacaactatcactgtacctcccaacatctgtttttttcactttaacataaaaaacaaccttttaacattaaaaacccaatatttatttatttgtttggacaatggacactggacacctaggggggaggtcgtagtacccccctatgttttctcccctaaataaccccaaaaatctaagaaaaaaagacctcaaaaaggtctttaattaacatctcaaatttcgcatttattccaatttcctttttgcgtgtgatgcgagctcatcggctccgtcgatactatgttatacgccaactttcaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtgactgcagtgagatctggtaatgactctctagcttgaggcatcaaataaaacgaaaggctcagtcgaaagactgggcctttcgttttatctgttgtttgtcggtgaacgctctcctgagtaggacaaatccgccgctctagctaagcagaaggccatcctgacggatggcctttttgcgtttctacaaactcttgttaactctagagctgcctgccgcgtttcggtgatgaagatcttcccgatgattaattaattcagaacgctcggttgccgccgggcgttttttatgaagcttcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgaca

SEQ ID NO: 63 - DNA, 119 bp, Bacillus subtilus
ccggaaaaaatgtaatcacgatcaaaaggacaaagtcttcgggctttgtcctttttttatgagaaaaacgtgtgatgtaattcacaatcctgtttggctagtttttgtatgataagact

SEQ ID NO: 64 - DNA, 129 bp, Bacillus subtilus
ccaggcatcaaataaaacgaaaggctcagtcgaaagactgggcctttcgttttatctgttgtttgtcggtgaacgctctctactagagtcacactggctcaccttcgggtgggcctttctgcgtttata

SEQ ID NO: 65 - DNA, 544 bp, Eschericha coli
atggcgggatcgttgtatatttcttgacaccttttcggcatcgccctaaattcggcgtcctcatattgtgtgaggacgttttattacgtgtttacgaagcaaaagctaaaaccaggagctatttaatggcaacagttaaccagctggtacgcaaaccacgtgctcgcaaagttgcgaaaagcaacgtgcctgcgctggaagcatgcccgcaaaaacgtggcgtatgtactcgtgtatatactaccactcctaaaaaaccgaactccgcgctgcgtaaagtatgccgtgttcgtctgactaacggtttcgaagtgacttcctacatcggtggtgaaggtcacaacctgcaggagcactccgtgatcctgatccgtggcggtcgtgttaaagacctcccgggtgttcgttaccacaccgtacgtggtgcgcttgactgctccggcgttaaagaccgtaagcaggctcgttccaagtatggcgtgaagcgtcctaaggcttaggttaataacaggcctgctggtaatcgcaggcctttttattttta

SEQ ID NO: 66 - DNA, 76 bp, Streptococcus pyogenes
gttttagagctagaaatagcaagttaaaataaggctagtccgttatcaacttgaaaaagtggcaccgagtcggtgc

SEQ ID NO: 67 - DNA, 95 bp, lambda phage
gactcctgttgatagatccagtaatgacctcagaactccatctggatttgttcagaacgctcggttgccgccgggcgttttttattggtgagaat

SEQ ID NO: 68 - DNA, 1491 bp, Bacillus subtilus
atgaatagtctatcattggtgttctggagtattttagcagttgttggattactgttatttattaaattcaaacccccaacaattgcttcactactcttaagcaaagatgaggcaaaagaaataagcattcaatttataaaagagtttgttgggatagatgtagagaactgggatttttattcagtatattggtatgaccacgatacagtaaataaacttcatcacttaggcatacttaagaaaaatagaaaggttttatatgatgttgggttggtcgaatcatggagagtccgtttcgttcaccagaatcaatcatttgtagttggtgtcaatgccaatcgagaaatcacttttttttatgcggatgttccgaaaaaaaccctttcggggaagtttgaacaagtttctccagagacactcaagcagaggttaatggcttcacctgatggactttggtctagagcaaatatgactggtactggtaaaaaagaggaggattttcgcgaggtcagtacttattggtacatagcggaagcgggagatattcggctcaaagtgactgttgaattacagggcggccgaatttcttatattggtactgaacaagaaatactaacagatcaaatgagtaaagtcattcgagatgaacaagtggaatcgacattcggagtatctggtatgctgggttcagctttagcgatgatccttgcgattctcatccttgtatttatggatgtgcaaacaagcataatcttcagtcttgttctgggtttgttgattataatatgccagtcattgacgctgaaagaagatattcaattaacaattgtaaatgcttatgatgcaagaatgagtgtcaaaacggtcagtttattaggtattttgtctacacttcttacaggattattaacaggatttgtagtatttatatgttcattggcaggaaatgcgcttgctggtgattttggatggaaaacgtttgaacaaccaatagttcagattttctatggaataggagcagggctcattagtttaggagtgacttctctgctgtttaacttattggagaaaaagcaatatttacgaatttcacctgagctttctaaccgaactgtctttctatcaggttttacctttaggcaaggattgaatatgagcatacaaagttcaattggagaagaggtcatctatcggctattaatgattccagtcatttggtggatgagtggaaatatcctcatctccattattgtatcttcctttttatgggcggtgatgcaccaagtaactggatatgacccaaggtggatacgttggctgcatctatttatattcggttgctttctgggagttctcttcatcaaatttggttttatttgtgtattagtagctcatttcattcataatttagtactcgtctgtatgccgctgtggcagttcaagcttcagaaacatatgcatcatgatcagccaaagcatacttcactctaa

SEQ ID NO: 69 - DNA, 23 bp, Bacillus subtilus
tggctgcatctatttatattcgg

SEQ ID NO: 70 - DNA, 20 bp, Bacillus subtilus
tggctgcatctatttatatt

SEQ ID NO: 71 - DNA, 23 bp, Bacillus subtilus
tggctgcatctatttatattcgg

SEQ ID NO: 72 - RNA, 96 nt, Artificial Sequence
  synthesized sequence
uggcugcaucuauuuauauuguuuuagagcuagaaauagcaaguuaaaauaaggcuaguccguuaucaacuugaaaaaguggcaccgagucggugc

SEQ ID NO: 73 - DNA, 96 bp, Artificial Sequence; identical to SEQ ID NO: 28
  synthesized sequence

SEQ ID NO: 74 - DNA, 93 bp, Bacillus subtilus
tcgctgataaacagctgacatcaactaaaagcttcattaaatactttgaaaaaagttgttgacttaaaagaagctaaatgttatagtaataaa

SEQ ID NO: 75 - DNA, 215 bp, Artificial Sequence
  synthesized sequence
ccggaaaaaatgtaatcacgatcaaaaggacaaagtcttcgggctttgtcctttttttatgagaaaaacgtgtgatgtaattcacaatcctgtttggctagtttttgtatgataagacttggctgcatctatttatattgttttagagctagaaatagcaagttaaaataaggctagtccgttatcaacttgaaaaagtggcaccgagtcggtgc

SEQ ID NO: 76 - DNA, 8639 bp, Artificial Sequence
  synthesized sequence
gaattcctccattttcttctgctatcaaaataacagactcgtgattttccaaacgagctttcaaaaaagcctctgccccttgcaaatcggatgcctgtctataaaattcccgatattggttaaacagcggcgcaatggcggccgcatctgatgtctttgcttggcgaatgttcatcttatttcttcctccctctcaataattttttcattctatcccttttctgtaaagtttatttttcagaatacttttatcatcatgctttgaaaaaatatcacgataatatccattgttctcacggaagcacacgcaggtcatttgaacgaattttttcgacaggaatttgccgggactcaggagcatttaacctaaaaaagcatgacatttcagcataatgaacatttactcatgtctattttcgttcttttctgtatgaaaatagttatttcgagtctctacggaaatagcgagagatgatatacctaaatagagataaaatcatctcaaaaaaatgggtctactaaaatattattccatctattacaataaattcacagaatagtcttttaagtaagtctactctgaatttttttaaaaggagagggtaactagtggccccaaaaaagaaacgcaaggttatggataaaaaatacagcattggtctggatatcggaaccaacagcgttgggtgggcagtaataacagatgaatacaaagtgccgtcaaaaaaatttaaggttctggggaatacagatcgccacagcataaaaaagaatctgattggggcattgctgtttgattcgggtgagacagctgaggccacgcgtctgaaacgtacagcaagaagacgttacacacgtcgtaaaaatcgtatttgctacttacaggaaattttttctaacgaaatggccaaggtagatgatagtttcttccatcgtctcgaagaatcttttctggttgaggaagataaaaaacacgaacgtcaccctatctttggcaatatcgtggatgaagtggcctatcatgaaaaataccctacgatttatcatcttcgcaagaagttggttgatagtacggacaaagcggatctgcgtttaatctatcttgcgttagcgcacatgatcaaatttcgtggtcatttcttaattgaaggtgatctgaatcctgataactctgatgtggacaaattgtttatacaattagtgcaaacctataatcagctgttcgaggaaaaccccattaatgcctctggagttgatgccaaagcgattttaagcgcgagactttctaagtcccggcgtctggagaatctgatcgcccagttaccaggggaaaagaaaaatggtctgtttggtaatctgattgccctcagtctggggcttaccccgaacttcaaatccaattttgacctggctgaggacgcaaagctgcagctgagcaaagatacttatgatgatgacctcgacaatctgctcgcccagattggtgaccaatatgcggatctgtttctggcagcgaagaatctttcggatgctatcttgctgtcggatattctgcgtgttaataccgaaatcaccaaagcgcctctgtctgcaagtatgatcaagagatacgacgagcaccaccaggacctgactcttcttaaggcactggtacgccaacagcttccggagaaatacaaagaaatattcttcgaccagtccaagaatggttacgcgggctacatcgatggtggtgcatcacaggaagagttctataaatttattaaaccaatccttgagaaaatggatggcacggaagagttacttgttaaacttaaccgcgaagacttgcttagaaagcaacgtacattcgacaacggctccatcccacaccagattcatttaggtgaacttcacgccatcttgcgcagacaagaagatttctatcccttcttaaaagacaatcgggagaaaatcgagaagatcctgacgttccgcattccctattatgtcggtcccctggcacgtggtaattctcggtttgcctggatgacgcgcaaaagtgaggaaaccatcaccccttggaactttgaagaagtcgtggataaaggtgctagcgcgcagtcttttatagaaagaatgacgaacttcgataaaaacttgcccaacgaaaaagtcctgcccaagcactctcttttatatgagtactttactgtgtacaacgaactgactaaagtgaaatacgttacggaaggtatgcgcaaacctgcctttcttagtggcgagcagaaaaaagcaattgtcgatcttctctttaaaacgaatcgcaaggtaactgtaaaacagctgaaggaagattatttcaaaaagatcgaatgctttgattctgtcgagatctcgggtgtcgaagatcgtttcaacgcttccttagggacctatcatgatttgctgaagataataaaagacaaagactttctcgacaatgaagaaaatgaagatattctggaggatattgttttgaccttgaccttattcgaagatagagagatgatcgaggagcgcttaaaaacctatgcccacctgtttgatgacaaagtcatgaagcaattaaagcgccgcagatatacggggtggggccgcttgagccgcaagttgattaacggtattagagacaagcagagcggaaaaactatcctggatttcctcaaatctgacggatttgcgaaccgcaattttatgcagcttatacatgatgattcgcttacattcaaagaggatattcagaaggctcaggtgtctgggcaaggtgattcactccacgaacatatagcaaatttggccggctctcctgcgattaagaaggggatcctgcaaacagttaaagttgtggatgaacttgtaaaagtaatgggccgccacaagccggagaatatcgtgatagaaatggcgcgcgagaatcaaacgacacaaaaaggtcaaaagaactcaagagagagaatgaagcgcattgaggaggggataaaggaacttggatctcaaattctgaaagaacatccagttgaaaacactcagctgcaaaatgaaaaattgtacctgtactacctgcagaatggaagagacatgtacgtggatcaggaattggatatcaatagactctcggactatgacgtagatcacattgtccctcagagcttcctcaaggatgattctatagataataaagtacttacgagatcggacaaaaatcgcggtaaatcggataacgtcccatcggaggaagtcgttaaaaagatgaaaaactattggcgtcaactgctgaacgccaagctgatcacacagcgtaagtttgataatctgactaaagccgaacgcggtggtcttagtgaactcgataaagcaggatttataaaacggcagttagtagaaacgcgccaaattacgaaacacgtggctcagatcctcgattctagaatgaatacaaagtacgatgaaaacgataaactgatccgtgaagtaaaagtcattaccttaaaatctaaacttgtgtccgatttccgcaaagattttcagttttacaaggtccgggaaatcaataactatcaccatgcacatgatgcatatttaaatgcggttgtaggcacggcccttattaagaaataccctaaactcgaaagtgagtttgtttatggggattataaagtgtatgacgttcgcaaaatgatcgcgaaatcagaacaggaaatcggtaaggctaccgctaaatactttttttattccaacattatgaatttttttaagaccgaaataactctcgcgaatggtgaaatccgtaaacggcctcttatagaaaccaatggtgaaacgggagaaatcgtttgggataaaggtcgtgactttgccaccgttcgtaaagtcctctcaatgccgcaagttaacattgtcaagaagacggaagttcaaacagggggattctccaaagaatctatcctgccgaagcgtaacagtgataaacttattgccagaaaaaaagattgggatccaaaaaaatacggaggctttgattcccctaccgtcgcgtatagtgtgctggtggttgctaaagtcgagaaagggaaaagcaagaaattgaaatcagttaaagaactgctgggtattacaattatggaaagatcgtcctttgagaaaaatccgatcgactttttagaggccaaggggtataaggaagtgaaaaaagatctcatcatcaaattaccgaagtatagtctttttgagctggaaaacggcagaaaaagaatgctggcctccgcgggcgagttacagaagggaaatgagctggcgctgccttccaaatatgttaattttctgtaccttgccagtcattatgagaaactgaagggcagccccgaagataacgaacagaaacaattattcgtggaacagcataagcactatttagatgaaattatagagcaaattagtgaattttctaagcgcgttatcctcgcggatgctaatttagacaaagtactgtcagcttataataaacatcgggataagccgattagagaacaggccgaaaatatcattcatttgtttaccttaaccaaccttggagcaccagctgccttcaaatatttcgataccacaattgatcgtaaacggtatacaagtacaaaagaagtcttggacgcaaccctcattcatcaatctattactggattatatgagacacgcattgatctttcacagctgggcggagacaagaagaaaaaactgaaactgcaccatcatcaccatcatcatcaccatcattgataactcgagaaagcttacataaaaaaccggccttggccccgccggttttttattatttttcttcctccgcatgttcaatccgctccataatcgacggatggctccctctgaaaattttaacgagaaacggcgggttgacccggctcagtcccgtaacggccaagtcctgaaacgtctcaatcgccgcttcccggtttccggtcagctcaatgccgtaacggtcggcggcgttttcctgataccgggagacggcattcgtaatcgaattcgcggccgcacatggccggaaaaaatgtaatcacgatcaaaaggacaaagtcttcgggctttgtcctttttttatgagaaaaacgtgtgatgtaattcacaatcctgtttggctagtttttgtatgataagacttggctgcatctatttatattgttttagagctagaaatagcaagttaaaataaggctagtccgttatcaacttgaaaaagtggcaccgagtcggtgcgactcctgttgatagatccagtaatgacctcagaactccatctggatttgttcagaacgctcggttgccgccgggcgttttttattggtgagaatgtcgacctcgagagttacgctagggataacagggtaatataggagctccagtcggcttaaaccagttttcgctggtgcgaaaaaagagtgtcttgtgacacctaaattcaaaatctatcggtcagatttataccgatttgattttatatattcttgaataacatacgccgagttatcacataaaagcgggaaccaatcataaaatttaaacttcattgcataatccattaaactcttaaattctacgattccttgttcatcaataaactcaatcatttctttaattaatttatatctatctgttgttgttttctttaataattcattaacatctacaccgccataaactatcatatcttctttttgatatttaaatttattaggatcgtccatgtgaagcatatatctcacaagacctttcacacttcctgcaatctgcggaatagtcgcattcaattcttctgttaattatttttatctgttcataagatttattaccctcatacatcactagaatatgataatgctcttttttcatcctaccttctgtatcagtatccctatcatgtaatggagacactacaaattgaatgtgtaactcttttaaatactctaaccactcggcttttgctgattctggatataaaacaaatgtccaattacgtcctcttgaatttttcttgttttcagtttcttttattacattttcgctcatgatataataacggtgctaatacacttaacaaaatttagtcatagataggcagcatgccagtgctgtctatctttttttgtttaaaatgcaccgtattcctcctttgcatatttttttattagaataccggttgcatctgatttgctaatattatatttttctttgattctatttaatatctcattttcttctgttgtaagtcttaaagtaacagcaacttttttctcttcttttctatctacaactatcactgtacctcccaacatctgtttttttcactttaacataaaaaacaaccttttaacattaaaaacccaatatttatttatttgtttggacaatggacactggacacctaggggggaggtcgtagtacccccctatgttttctcccctaaataaccccaaaaatctaagaaaaaaagacctcaaaaaggtctttaattaacatctcaaatttcgcatttattccaatttcctttttgcgtgtgatgcgagctcatcggctccgtcgatactatgttatacgccaactttcaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtgactgcagtgagatctggtaatgactctctagcttgaggcatcaaataaaacgaaaggctcagtcgaaagactgggcctttcgttttatctgttgtttgtcggtgaacgctctcctgagtaggacaaatccgccgctctagctaagcagaaggccatcctgacggatggcctttttgcgtttctacaaactcttgttaactctagagctgcctgccgcgtttcggtgatgaagatcttcccgatgattaattaattcagaacgctcggttgccgccgggcgttttttatgaagcttcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgaca

SEQ ID NO: 77 - DNA, 45 bp, Artificial Sequence
  synthesized sequence
tggctgcatctatttatattgttttagagctagaaatagcaagtt

SEQ ID NO: 78 - DNA, 44 bp, Artificial Sequence
  synthesized sequence
aatataaatagatgcagccaagtcttatcatacaaaaactagcc

SEQ ID NO: 79 - DNA, 1070 bp, Bacillus subtilus
gcatactttgaatgtctattttggtcagagactgcatctaaaatcaagacgggagtttcctttggcgcaggcagattcacagatttgctgccgttttggtaagactgtaaaagctcatgctccatatctgcattaaaaaattgcttaaagttaatggctttatagatttctttttcttcatcagttaaaaacgactgtcgaatcacctcaggattatatacagcggaaggtataaaccgatgaaaaccaatcgaggttaacaggtggaaccctctcactttcaatcggtcaactccgctcaatttatacgtgacgtactgctggggcaatccaatatccatcgcaataatggccttgatttccttaggatatttctgtgcccaatacatcgcttcaatcccggatatcgaatgaggcattaaaatataaggaggcttatttccgcttttcataagcgctttcctcgtctgttccaataccgtatcaatatctctgtcatcgtgagacacttcactgtatccataacctgcccgatctacaacagcaatcttattttcttttgaaaacttgctgtacagccccttcatttcataagcaggcgcagcaatacccgaaccggacataaacacaaacgtatccttcccgcttccctcttgatacacattcatctttttaccgtcaacatcgactactgtgcctttacctttcagcagtgccgcctccttatttagctggaaatggtgataaataaataccgagacggatacaagcaaaaccaaagcagccaagctgacaaaaacaattttgaggactttccataatgttttcatatgatgctcctttcacttgataccgaaggatacaatatgaaaataaatttctgattaactttggaacagttttttttcacatttgactttgcccttacggaaaggtgtacatttggagcatagcagaacatttgatgaatttacctactaataaaaaaatatttcattaaaaaaccttcctgcttataggaaggaccgtttcatcataatattcaaccgtttgtcgcacatacaagacgg

SEQ ID NO: 80 - DNA, 254 bp, Bacillus amyloliquefaciens
tctagatacataaaaaaccggccttggccccgccggttttttattatttttcttcctccgcatgttcaatccgctccataatcgacggatggctccctctgaaaattttaacgagaaacggcgggttgacccggctcagtcccgtaacggccaagtcctgaaacgtctcaatcgccgcttcccggtttccggtcagctcaatgccgtaacggtcggcggcgttttcctgataccgggagacggcattcgtaatc

SEQ ID NO: 81 - DNA, 995 bp, Bacillus subtilus
ggagctcacccctaattttccacttatgttttgaatattttcttgttctataaacaaaacgtcaaaaagacggtccattatgttataaaaattctgatgcctaatagattgacaaaaaatctcttcgaggaaattaaatcaagagattattttcaaataaactttagtgattaagaaagcaatgactttggttaaatgaatgaagtctcctgtaatttaaatcaaatatctatactaaaaaccccttcggctaaaatatccagaggggttaaacatcttactccacagtaacactcttcattacgctgcttatcgacatcacaaccgagatgcagggcagcattagtaagcaatcagctgcaatcgacaacggaaacagagcaagcgctgtgtttacttccggcaatacgaatctgtcatccgcattgtctaggcctttcagcgagatgatgcaagtgtttactccccaagtggcaacttccttgacgtttacgcggatgttcaggtttacgttctcttgtgtttccagtgttcgatcaaggcaatcgtactgtacttcagctcgccgcggcaaagccttaagaaatctcattcagcttcagtgcaccttcgacacatacagaacgatgtcggcccttggaagactcatgcatggactgactacgctaaaaaaaacacccgcttgtataacgagcggatgttagaactttcgaaattattgattgatatcttttagcgcttgtttcggttgcgaattgaaaatcagcaatgatgcaataattgcaatcataacccctaacgcaccaacccaggtaatcgaggccaatgatacgttttccacaaaaacccctcctatacctgcgccgaccgccatggcgaattgcatcattgactgattcatgctaagcaaaacacctgacatttccggttctattgtagccaggtgaaattgctgtgtcggaccggtggaccatgcggcaaacgaccataatatg

SEQ ID NO: 82 - DNA, 576 bp, Bacillus subtilus
atgagtcagaaaacagacgcacctttagaatcgtatgaagtgaacggcgcaacaattgccgtgctgccagaagaaatagacggcaaaatctgttccaaaattattgaaaaagattgcgtgttttatgtaaacatgaagccgctgcaaattgtcgacagaagctgccgattttttggatcaagctatgcgggaagaaaagcaggaacttatgaagtgacaaaaatttcacacaagccgccgatcatggtggacccttcgaaccaaatctttttattccctacactttcttcgacaagaccccaatgcggctggatttcccatgtgcatgtaaaagaattcaaagcgactgaattcgacgatacggaagtgacgttttccaatgggaaaacgatggagctgccgatctcttataattcgttcgagaaccaggtataccgaacagcgtggctcagaaccaaattccaagacagaatcgaccaccgcgtgccgaaaagacaggaatttatgctgtacccgaaagaagagcggacgaagatgatttatgattttattttgcgtgagctcggggaacggtat

SEQ ID NO: 83 - DNA, 9286 bp, Artificial Sequence
  synthesized sequence
gaattcctccattttcttctgctatcaaaataacagactcgtgattttccaaacgagctttcaaaaaagcctctgccccttgcaaatcggatgcctgtctataaaattcccgatattggttaaacagcggcgcaatggcggccgcatctgatgtctttgcttggcgaatgttcatcttatttcttcctccctctcaataattttttcattctatcccttttctgtaaagtttatttttcagaatacttttatcatcatgctttgaaaaaatatcacgataatatccattgttctcacggaagcacacgcaggtcatttgaacgaattttttcgacaggaatttgccgggactcaggagcatttaacctaaaaaagcatgacatttcagcataatgaacatttactcatgtctattttcgttcttttctgtatgaaaatagttatttcgagtctctacggaaatagcgagagatgatatacctaaatagagataaaatcatctcaaaaaaatgggtctactaaaatattattccatctattacaataaattcacagaatagtcttttaagtaagtctactctgaatttttttaaaaggagagggtaactagtggccccaaaaaagaaacgcaaggttatggataaaaaatacagcattggtctggatatcggaaccaacagcgttgggtgggcagtaataacagatgaatacaaagtgccgtcaaaaaaatttaaggttctggggaatacagatcgccacagcataaaaaagaatctgattggggcattgctgtttgattcgggtgagacagctgaggccacgcgtctgaaacgtacagcaagaagacgttacacacgtcgtaaaaatcgtatttgctacttacaggaaattttttctaacgaaatggccaaggtagatgatagtttcttccatcgtctcgaagaatcttttctggttgaggaagataaaaaacacgaacgtcaccctatctttggcaatatcgtggatgaagtggcctatcatgaaaaataccctacgatttatcatcttcgcaagaagttggttgatagtacggacaaagcggatctgcgtttaatctatcttgcgttagcgcacatgatcaaatttcgtggtcatttcttaattgaaggtgatctgaatcctgataactctgatgtggacaaattgtttatacaattagtgcaaacctataatcagctgttcgaggaaaaccccattaatgcctctggagttgatgccaaagcgattttaagcgcgagactttctaagtcccggcgtctggagaatctgatcgcccagttaccaggggaaaagaaaaatggtctgtttggtaatctgattgccctcagtctggggcttaccccgaacttcaaatccaattttgacctggctgaggacgcaaagctgcagctgagcaaagatacttatgatgatgacctcgacaatctgctcgcccagattggtgaccaatatgcggatctgtttctggcagcgaagaatctttcggatgctatcttgctgtcggatattctgcgtgttaataccgaaatcaccaaagcgcctctgtctgcaagtatgatcaagagatacgacgagcaccaccaggacctgactcttcttaaggcactggtacgccaacagcttccggagaaatacaaagaaatattcttcgaccagtccaagaatggttacgcgggctacatcgatggtggtgcatcacaggaagagttctataaatttattaaaccaatccttgagaaaatggatggcacggaagagttacttgttaaacttaaccgcgaagacttgcttagaaagcaacgtacattcgacaacggctccatcccacaccagattcatttaggtgaacttcacgccatcttgcgcagacaagaagatttctatcccttcttaaaagacaatcgggagaaaatcgagaagatcctgacgttccgcattccctattatgtcggtcccctggcacgtggtaattctcggtttgcctggatgacgcgcaaaagtgaggaaaccatcaccccttggaactttgaagaagtcgtggataaaggtgctagcgcgcagtcttttatagaaagaatgacgaacttcgataaaaacttgcccaacgaaaaagtcctgcccaagcactctcttttatatgagtactttactgtgtacaacgaactgactaaagtgaaatacgttacggaaggtatgcgcaaacctgcctttcttagtggcgagcagaaaaaagcaattgtcgatcttctctttaaaacgaatcgcaaggtaactgtaaaacagctgaaggaagattatttcaaaaagatcgaatgctttgattctgtcgagatctcgggtgtcgaagatcgtttcaacgcttccttagggacctatcatgatttgctgaagataataaaagacaaagactttctcgacaatgaagaaaatgaagatattctggaggatattgttttgaccttgaccttattcgaagatagagagatgatcgaggagcgcttaaaaacctatgcccacctgtttgatgacaaagtcatgaagcaattaaagcgccgcagatatacggggtggggccgcttgagccgcaagttgattaacggtattagagacaagcagagcggaaaaactatcctggatttcctcaaatctgacggatttgcgaaccgcaattttatgcagcttatacatgatgattcgcttacattcaaagaggatattcagaaggctcaggtgtctgggcaaggtgattcactccacgaacatatagcaaatttggccggctctcctgcgattaagaaggggatcctgcaaacagttaaagttgtggatgaacttgtaaaagtaatgggccgccacaagccggagaatatcgtgatagaaatggcgcgcgagaatcaaacgacacaaaaaggtcaaaagaactcaagagagagaatgaagcgcattgaggaggggataaaggaacttggatctcaaattctgaaagaacatccagttgaaaacactcagctgcaaaatgaaaaattgtacctgtactacctgcagaatggaagagacatgtacgtggatcaggaattggatatcaatagactctcggactatgacgtagatcacattgtccctcagagcttcctcaaggatgattctatagataataaagtacttacgagatcggacaaaaatcgcggtaaatcggataacgtcccatcggaggaagtcgttaaaaagatgaaaaactattggcgtcaactgctgaacgccaagctgatcacacagcgtaagtttgataatctgactaaagccgaacgcggtggtcttagtgaactcgataaagcaggatttataaaacggcagttagtagaaacgcgccaaattacgaaacacgtggctcagatcctcgattctagaatgaatacaaagtacgatgaaaacgataaactgatccgtgaagtaaaagtcattaccttaaaatctaaacttgtgtccgatttccgcaaagattttcagttttacaaggtccgggaaatcaataactatcaccatgcacatgatgcatatttaaatgcggttgtaggcacggcccttattaagaaataccctaaactcgaaagtgagtttgtttatggggattataaagtgtatgacgttcgcaaaatgatcgcgaaatcagaacaggaaatcggtaaggctaccgctaaatactttttttattccaacattatgaatttttttaagaccgaaataactctcgcgaatggtgaaatccgtaaacggcctcttatagaaaccaatggtgaaacgggagaaatcgtttgggataaaggtcgtgactttgccaccgttcgtaaagtcctctcaatgccgcaagttaacattgtcaagaagacggaagttcaaacagggggattctccaaagaatctatcctgccgaagcgtaacagtgataaacttattgccagaaaaaaagattgggatccaaaaaaatacggaggctttgattcccctaccgtcgcgtatagtgtgctggtggttgctaaagtcgagaaagggaaaagcaagaaattgaaatcagttaaagaactgctgggtattacaattatggaaagatcgtcctttgagaaaaatccgatcgactttttagaggccaaggggtataaggaagtgaaaaaagatctcatcatcaaattaccgaagtatagtctttttgagctggaaaacggcagaaaaagaatgctggcctccgcgggcgagttacagaagggaaatgagctggcgctgccttccaaatatgttaattttctgtaccttgccagtcattatgagaaactgaagggcagccccgaagataacgaacagaaacaattattcgtggaacagcataagcactatttagatgaaattatagagcaaattagtgaattttctaagcgcgttatcctcgcggatgctaatttagacaaagtactgtcagcttataataaacatcgggataagccgattagagaacaggccgaaaatatcattcatttgtttaccttaaccaaccttggagcaccagctgccttcaaatatttcgataccacaattgatcgtaaacggtatacaagtacaaaagaagtcttggacgcaaccctcattcatcaatctattactggattatatgagacacgcattgatctttcacagctgggcggagacaagaagaaaaaactgaaactgcaccatcatcaccatcatcatcaccatcattgataactcgagaaagcttacataaaaaaccggccttggccccgccggttttttattatttttcttcctccgcatgttcaatccgctccataatcgacggatggctccctctgaaaattttaacgagaaacggcgggttgacccggctcagtcccgtaacggccaagtcctgaaacgtctcaatcgccgcttcccggtttccggtcagctcaatgccgtaacggtcggcggcgttttcctgataccgggagacggcattcgtaatcgaattcgcggccgcacgcgtcatggtcgctgataaacagctgacatcaactaaaagcttcattaaatactttgaaaaaagttgttgacttaaaagaagctaaatgttatagtaataaaagcaggtgccaggcatcaaataaaacgaaaggctcagtcgaaagactgggcctttcgttttatctgttgtttgtcggtgaacgctctctactagagtcacactggctcaccttcgggtgggcctttctgcgtttataatggcgggatcgttgtatatttcttgacaccttttcggcatcgccctaaattcggcgtcctcatattgtgtgaggacgttttattacgtgtttacgaagcaaaagctaaaaccaggagctatttaatggcaacagttaaccagctggtacgcaaaccacgtgctcgcaaagttgcgaaaagcaacgtgcctgcgctggaagcatgcccgcaaaaacgtggcgtatgtactcgtgtatatactaccactcctaaaaaaccgaactccgcgctgcgtaaagtatgccgtgttcgtctgactaacggtttcgaagtgacttcctacatcggtggtgaaggtcacaacctgcaggagcactccgtgatcctgatccgtggcggtcgtgttaaagacctcccgggtgttcgttaccacaccgtacgtggtgcgcttgactgctccggcgttaaagaccgtaagcaggctcgttccaagtatggcgtgaagcgtcctaaggcttaggttaataacaggcctgctggtaatcgcaggcctttttatttttacacctgcgttttagagctagaaatagcaagttaaaataaggctagtccgttatcaacttgaaaaagtggcaccgagtcggtgcgactcctgttgatagatccagtaatgacctcagaactccatctggatttgttcagaacgctcggttgccgccgggcgttttttattggtgagaatgtcgacctcgagagttacgctagggataacagggtaatataggagctccagtcggcttaaaccagttttcgctggtgcgaaaaaagagtgtcttgtgacacctaaattcaaaatctatcggtcagatttataccgatttgattttatatattcttgaataacatacgccgagttatcacataaaagcgggaaccaatcataaaatttaaacttcattgcataatccattaaactcttaaattctacgattccttgttcatcaataaactcaatcatttctttaattaatttatatctatctgttgttgttttctttaataattcattaacatctacaccgccataaactatcatatcttctttttgatatttaaatttattaggatcgtccatgtgaagcatatatctcacaagacctttcacacttcctgcaatctgcggaatagtcgcattcaattcttctgttaattatttttatctgttcataagatttattaccctcatacatcactagaatatgataatgctcttttttcatcctaccttctgtatcagtatccctatcatgtaatggagacactacaaattgaatgtgtaactcttttaaatactctaaccactcggcttttgctgattctggatataaaacaaatgtccaattacgtcctcttgaatttttcttgttttcagtttcttttattacattttcgctcatgatataataacggtgctaatacacttaacaaaatttagtcatagataggcagcatgccagtgctgtctatctttttttgtttaaaatgcaccgtattcctcctttgcatatttttttattagaataccggttgcatctgatttgctaatattatatttttctttgattctatttaatatctcattttcttctgttgtaagtcttaaagtaacagcaacttttttctcttcttttctatctacaactatcactgtacctcccaacatctgtttttttcactttaacataaaaaacaaccttttaacattaaaaacccaatatttatttatttgtttggacaatggacactggacacctaggggggaggtcgtagtacccccctatgttttctcccctaaataaccccaaaaatctaagaaaaaaagacctcaaaaaggtctttaattaacatctcaaatttcgcatttattccaatttcctttttgcgtgtgatgcgagctcatcggctccgtcgatactatgttatacgccaactttcaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtgactgcagtgagatctggtaatgactctctagcttgaggcatcaaataaaacgaaaggctcagtcgaaagactgggcctttcgttttatctgttgtttgtcggtgaacgctctcctgagtaggacaaatccgccgctctagctaagcagaaggccatcctgacggatggcctttttgcgtttctacaaactcttgttaactctagagctgcctgccgcgtttcggtgatgaagatcttcccgatgattaattaattcagaacgctcggttgccgccgggcgttttttatgaagcttcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgaca

SEQ ID NO: 84 - DNA, 1119 bp, Bacillus subtilus
atgcgcaagtggattgcggcagcaggacttgcttacgtgctgtacgggctgtttttttattggtattttttcctgtcgggtgattccgcaataccggaagccgtgaaagggacgcaggctgatccggcttctttcatgaagccgtctgagttggcagtggccgagcagtattcgaatgtcaagaattttttattttttatcggggtaccacttgattggtttctgttttttgttctgcttgtcagcggtgtttcaaagaaaatcaagaaatggatcgaagcggccgtgccttttcggtttttgcagaccgttggttttgtgtttgtgctttcgctgattacaacattggtgacgctgcctttagattggataggctatcaagtatcgcttgactataacatttccacacagacaacggccagctgggctaaggatcaggttatcagcttttggatcagctttccaatctttacgctttgcgttctcgttttttattggctgatcaaaaggcatgaaaaaaaatggtggttatacgcctggctgttaacagtgccgttttcgctgtttctgttttttattcagccggtcattatcgatcctttatacaatgatttttatccgctgaaaaacaaagagcttgaaagcaaaattttagagctggcagatgaagccaatattccggctgaccatgtatatgaagtgaacatgtcagaaaaaacaaatgcgctgaatgcctatgttacaggaattggggccaataaacggattgtattgtgggatacgacgctgaacaaacttgacgattcagaaattctgtttattatgggccacgaaatgggccattatgtcatgaagcacgtttacatcggtctggctggctatttgctcgtgtcgctcgccggattttatgtcattgataagctttacaagcggacggttcgcctaacccgcagcatgtttcatttagaagggcggcatgatcttgcggcacttccgctgttattgcttttgttttctgttttgagctttgcggttacgcctttttctaatgctgtctcgcgttatcaggagaataaggctgaccagtatgggatcgagttaacgcaaacaacaagctga

SEQ ID NO: 85 - DNA, 23 bp, Bacillus subtilus
tcaagctctttgtttttcagcgg

SEQ ID NO: 86 - DNA, 20 bp, Bacillus subtilus
tcaagctctttgtttttcag

SEQ ID NO: 87 - DNA, 23 bp, Bacillus subtilus
tcaagctctttgtttttcagcgg

SEQ ID NO: 88 - RNA, 96 nt, Artificial Sequence
  synthesized sequence
ucaagcucuuuguuuuucagguuuuagagcuagaaauagcaaguuaaaauaaggcuaguccguuaucaacuugaaaaaguggcaccgagucggugc

SEQ ID NO: 89 - DNA, 96 bp, Artificial Sequence
  synthesized sequence
tcaagctctttgtttttcaggttttagagctagaaatagcaagttaaaataaggctagtccgttatcaacttgaaaaagtggcaccgagtcggtgc

SEQ ID NO: 90 - DNA, 189 bp, Artificial Sequence
  synthesized sequence
tcgctgataaacagctgacatcaactaaaagcttcattaaatactttgaaaaaagttgttgacttaaaagaagctaaatgttatagtaataaatcaagctctttgtttttcaggttttagagctagaaatagcaagttaaaataaggctagtccgttatcaacttgaaaaagtggcaccgagtcggtgc

SEQ ID NO: 91 - DNA, 8618 bp, Artificial Sequence
  synthesized sequence
gaattcctccattttcttctgctatcaaaataacagactcgtgattttccaaacgagctttcaaaaaagcctctgccccttgcaaatcggatgcctgtctataaaattcccgatattggttaaacagcggcgcaatggcggccgcatctgatgtctttgcttggcgaatgttcatcttatttcttcctccctctcaataattttttcattctatcccttttctgtaaagtttatttttcagaatacttttatcatcatgctttgaaaaaatatcacgataatatccattgttctcacggaagcacacgcaggtcatttgaacgaattttttcgacaggaatttgccgggactcaggagcatttaacctaaaaaagcatgacatttcagcataatgaacatttactcatgtctattttcgttcttttctgtatgaaaatagttatttcgagtctctacggaaatagcgagagatgatatacctaaatagagataaaatcatctcaaaaaaatgggtctactaaaatattattccatctattacaataaattcacagaatagtcttttaagtaagtctactctgaatttttttaaaaggagagggtaactagtggccccaaaaaagaaacgcaaggttatggataaaaaatacagcattggtctggatatcggaaccaacagcgttgggtgggcagtaataacagatgaatacaaagtgccgtcaaaaaaatttaaggttctggggaatacagatcgccacagcataaaaaagaatctgattggggcattgctgtttgattcgggtgagacagctgaggccacgcgtctgaaacgtacagcaagaagacgttacacacgtcgtaaaaatcgtatttgctacttacaggaaattttttctaacgaaatggccaaggtagatgatagtttcttccatcgtctcgaagaatcttttctggttgaggaagataaaaaacacgaacgtcaccctatctttggcaatatcgtggatgaagtggcctatcatgaaaaataccctacgatttatcatcttcgcaagaagttggttgatagtacggacaaagcggatctgcgtttaatctatcttgcgttagcgcacatgatcaaatttcgtggtcatttcttaattgaaggtgatctgaatcctgataactctgatgtggacaaattgtttatacaattagtgcaaacctataatcagctgttcgaggaaaaccccattaatgcctctggagttgatgccaaagcgattttaagcgcgagactttctaagtcccggcgtctggagaatctgatcgcccagttaccaggggaaaagaaaaatggtctgtttggtaatctgattgccctcagtctggggcttaccccgaacttcaaatccaattttgacctggctgaggacgcaaagctgcagctgagcaaagatacttatgatgatgacctcgacaatctgctcgcccagattggtgaccaatatgcggatctgtttctggcagcgaagaatctttcggatgctatcttgctgtcggatattctgcgtgttaataccgaaatcaccaaagcgcctctgtctgcaagtatgatcaagagatacgacgagcaccaccaggacctgactcttcttaaggcactggtacgccaacagcttccggagaaatacaaagaaatattcttcgaccagtccaagaatggttacgcgggctacatcgatggtggtgcatcacaggaagagttctataaatttattaaaccaatccttgagaaaatggatggcacggaagagttacttgttaaacttaaccgcgaagacttgcttagaaagcaacgtacattcgacaacggctccatcccacaccagattcatttaggtgaacttcacgccatcttgcgcagacaagaagatttctatcccttcttaaaagacaatcgggagaaaatcgagaagatcctgacgttccgcattccctattatgtcggtcccctggcacgtggtaattctcggtttgcctggatgacgcgcaaaagtgaggaaaccatcaccccttggaactttgaagaagtcgtggataaaggtgctagcgcgcagtcttttatagaaagaatgacgaacttcgataaaaacttgcccaacgaaaaagtcctgcccaagcactctcttttatatgagtactttactgtgtacaacgaactgactaaagtgaaatacgttacggaaggtatgcgcaaacctgcctttcttagtggcgagcagaaaaaagcaattgtcgatcttctctttaaaacgaatcgcaaggtaactgtaaaacagctgaaggaagattatttcaaaaagatcgaatgctttgattctgtcgagatctcgggtgtcgaagatcgtttcaacgcttccttagggacctatcatgatttgctgaagataataaaagacaaagactttctcgacaatgaagaaaatgaagatattctggaggatattgttttgaccttgaccttattcgaagatagagagatgatcgaggagcgcttaaaaacctatgcccacctgtttgatgacaaagtcatgaagcaattaaagcgccgcagatatacggggtggggccgcttgagccgcaagttgattaacggtattagagacaagcagagcggaaaaactatcctggatttcctcaaatctgacggatttgcgaaccgcaattttatgcagcttatacatgatgattcgcttacattcaaagaggatattcagaaggctcaggtgtctgggcaaggtgattcactccacgaacatatagcaaatttggccggctctcctgcgattaagaaggggatcctgcaaacagttaaagttgtggatgaacttgtaaaagtaatgggccgccacaagccggagaatatcgtgatagaaatggcgcgcgagaatcaaacgacacaaaaaggtcaaaagaactcaagagagagaatgaagcgcattgaggaggggataaaggaacttggatctcaaattctgaaagaacatccagttgaaaacactcagctgcaaaatgaaaaattgtacctgtactacctgcagaatggaagagacatgtacgtggatcaggaattggatatcaatagactctcggactatgacgtagatcacattgtccctcagagcttcctcaaggatgattctatagataataaagtacttacgagatcggacaaaaatcgcggtaaatcggataacgtcccatcggaggaagtcgttaaaaagatgaaaaactattggcgtcaactgctgaacgccaagctgatcacacagcgtaagtttgataatctgactaaagccgaacgcggtggtcttagtgaactcgataaagcaggatttataaaacggcagttagtagaaacgcgccaaattacgaaacacgtggctcagatcctcgattctagaatgaatacaaagtacgatgaaaacgataaactgatccgtgaagtaaaagtcattaccttaaaatctaaacttgtgtccgatttccgcaaagattttcagttttacaaggtccgggaaatcaataactatcaccatgcacatgatgcatatttaaatgcggttgtaggcacggcccttattaagaaataccctaaactcgaaagtgagtttgtttatggggattataaagtgtatgacgttcgcaaaatgatcgcgaaatcagaacaggaaatcggtaaggctaccgctaaatactttttttattccaacattatgaatttttttaagaccgaaataactctcgcgaatggtgaaatccgtaaacggcctcttatagaaaccaatggtgaaacgggagaaatcgtttgggataaaggtcgtgactttgccaccgttcgtaaagtcctctcaatgccgcaagttaacattgtcaagaagacggaagttcaaacagggggattctccaaagaatctatcctgccgaagcgtaacagtgataaacttattgccagaaaaaaagattgggatccaaaaaaatacggaggctttgattcccctaccgtcgcgtatagtgtgctggtggttgctaaagtcgagaaagggaaaagcaagaaattgaaatcagttaaagaactgctgggtattacaattatggaaagatcgtcctttgagaaaaatccgatcgactttttagaggccaaggggtataaggaagtgaaaaaagatctcatcatcaaattaccgaagtatagtctttttgagctggaaaacggcagaaaaagaatgctggcctccgcgggcgagttacagaagggaaatgagctggcgctgccttccaaatatgttaattttctgtaccttgccagtcattatgagaaactgaagggcagccccgaagataacgaacagaaacaattattcgtggaacagcataagcactatttagatgaaattatagagcaaattagtgaattttctaagcgcgttatcctcgcggatgctaatttagacaaagtactgtcagcttataataaacatcgggataagccgattagagaacaggccgaaaatatcattcatttgtttaccttaaccaaccttggagcaccagctgccttcaaatatttcgataccacaattgatcgtaaacggtatacaagtacaaaagaagtcttggacgcaaccctcattcatcaatctattactggattatatgagacacgcattgatctttcacagctgggcggagacaagaagaaaaaactgaaactgcaccatcatcaccatcatcatcaccatcattgataactcgagaaagcttacataaaaaaccggccttggccccgccggttttttattatttttcttcctccgcatgttcaatccgctccataatcgacggatggctccctctgaaaattttaacgagaaacggcgggttgacccggctcagtcccgtaacggccaagtcctgaaacgtctcaatcgccgcttcccggtttccggtcagctcaatgccgtaacggtcggcggcgttttcctgataccgggagacggcattcgtaatcgaattcgcggccgcacgcgtcatggtcgctgataaacagctgacatcaactaaaagcttcattaaatactttgaaaaaagttgttgacttaaaagaagctaaatgttatagtaataaatcaagctctttgtttttcaggttttagagctagaaatagcaagttaaaataaggctagtccgttatcaacttgaaaaagtggcaccgagtcggtgcgactcctgttgatagatccagtaatgacctcagaactccatctggatttgttcagaacgctcggttgccgccgggcgttttttattggtgagaatgtcgacctcgagagttacgctagggataacagggtaatataggagctccagtcggcttaaaccagttttcgctggtgcgaaaaaagagtgtcttgtgacacctaaattcaaaatctatcggtcagatttataccgatttgattttatatattcttgaataacatacgccgagttatcacataaaagcgggaaccaatcataaaatttaaacttcattgcataatccattaaactcttaaattctacgattccttgttcatcaataaactcaatcatttctttaattaatttatatctatctgttgttgttttctttaataattcattaacatctacaccgccataaactatcatatcttctttttgatatttaaatttattaggatcgtccatgtgaagcatatatctcacaagacctttcacacttcctgcaatctgcggaatagtcgcattcaattcttctgttaattatttttatctgttcataagatttattaccctcatacatcactagaatatgataatgctcttttttcatcctaccttctgtatcagtatccctatcatgtaatggagacactacaaattgaatgtgtaactcttttaaatactctaaccactcggcttttgctgattctggatataaaacaaatgtccaattacgtcctcttgaatttttcttgttttcagtttcttttattacattttcgctcatgatataataacggtgctaatacacttaacaaaatttagtcatagataggcagcatgccagtgctgtctatctttttttgtttaaaatgcaccgtattcctcctttgcatatttttttattagaataccggttgcatctgatttgctaatattatatttttctttgattctatttaatatctcattttcttctgttgtaagtcttaaagtaacagcaacttttttctcttcttttctatctacaactatcactgtacctcccaacatctgtttttttcactttaacataaaaaacaaccttttaacattaaaaacccaatatttatttatttgtttggacaatggacactggacacctaggggggaggtcgtagtacccccctatgttttctcccctaaataaccccaaaaatctaagaaaaaaagacctcaaaaaggtctttaattaacatctcaaatttcgcatttattccaatttcctttttgcgtgtgatgcgagctcatcggctccgtcgatactatgttatacgccaactttcaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtgactgcagtgagatctggtaatgactctctagcttgaggcatcaaataaaacgaaaggctcagtcgaaagactgggcctttcgttttatctgttgtttgtcggtgaacgctctcctgagtaggacaaatccgccgctctagctaagcagaaggccatcctgacggatggcctttttgcgtttctacaaactcttgttaactctagagctgcctgccgcgtttcggtgatgaagatcttcccgatgattaattaattcagaacgctcggttgccgccgggcgttttttatgaagcttcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgaca

SEQ ID NO: 92 - DNA, 45 bp, Artificial Sequence
  synthesized sequence
ttcaggatttggccgtgacggttttagagctagaaatagcaagtt

SEQ ID NO: 93 - DNA, 50 bp, Artificial Sequence
  synthesized sequence
cgtcacggccaaatcctgaatttattactataacatttagcttcttttaa

SEQ ID NO: 94 - DNA, 987 bp, Bacillus subtilus
gcttttccggccagaccatctttgtaattaatgccggaataagcaactttaatcaggacaccatccttcggcaaatcctctgttgatatggttttcacatggactgaaacatcatcggcatttttttctgcctgcaaggcttgaaataacgttgacattcggcacactccttttcatttatatcgtaaccgaagaacgttcaaaaaaccaaatcatcaagccgccattttcacttcgccggcacattgagacaataatggacaaatccggtatcctcttcatagccgttttgctcatacaagcttcttgccttccggttgtggtgctcagtctgaagtgttaaacattttgccccgttttgccctgcataatcctttgcggcagaaagcagccggccgccggctccctttgtacgcgcatgaggaacgacaaataagtcatttaatatgtatatccttttcattgacacagaagaaaacgttggatagagctgggtaaagcctatgaattctccattttcttctgctatcaaaataacagactcgtgattttccaaacgagctttcaaaaaagcctctgccccttgcaaatcggatgcctgtctataaaattcccgatattggttaaacagcggcgcaatggcggccgcatctgatgtctttgcttggcgaatgttcatgaattctccattttcttctgctatcaaaataacagactcgtgattttccaaacgagctttcaaaaaagcctctgccccttgcaaatcggatgcctgtctataaaattcccgatattggttaaacagcggcgcaatggcggccgcatctgatgtctttgcttggcgaatgttcatcttatttcttcctccctctcaataattttttcattctatcccttttctgtaaagtttatttttcagaatacttttatcatcatgctttgaaaaaatatcacgataatatccattgttctcacggaagcacacgc

SEQ ID NO: 95 - DNA, 756 bp, Bacillus subtilus
atcaacgcctcactcctcacatcaacccgttacttctattgtaatcataaattcaaattcttagaaccaagctgtgttccgcacttttccacccttttaagcatggaaaccccgatcgctgggaaaactaacaatgtttggagtgatgcaaatgaaaaaaatagtggcagccatcgtggtaatcggtcttgtgtttatcgcatttttttatctttacagccgatcaggcgatgtgtatcaatcggtagacgcggatttgatcacactgtcttcaagcggccaggaagatatcgagattgaaaaaagacagcacgtcaaagatatgctggatattatgaatcagggaaaacaggtgaagacagaaaaaacatcagcccctgattacgaagggacaatcaagtttcataaagaccggtatgactcattcagactatggattgacggcagccagcaagccgtttttttgaaggatggcacatactacaaattaagcaaaaatgatacaaaggcgctgctaaatattattaaaaaagaagcaaaggattgaaaatgaaaaagcgaagctaaccgcttcgctttttcattttattggggcaaaatatctctcagtgcccgtctgagcattttccccgtcgcatttttcggaatatcgtcaagaaacgtaatggcggcaggccgcttgtattttgccagatgcttttcgcagtgctgcatgatgtcctcctctgttaccccagagcgtttcggcaccacata

(19) United States
(12) Patent Application Publication
(10) Pub. No.: US 2022/0162621 A1
(43) Pub. Date: May 26, 2022

(54) Methods For Polynucleotide Integration Into The Genome Of Bacillus Using Dual Circular Recombinant DNA Constructs And Compositions Thereof

(71) Applicant: DANISCO US INC, Palo Alto, CA (US)

(72) Inventors: Wonchul SUH, HOCKESSIN, DE (US); Ryan L. FRISCH, Newark, DE (US); Stacey Irene Robida STUBBS, WOOLWICH TOWNSHIP, NJ (US); Derek Joseph ZIMMER, Blackwood, NJ (US)

(21) Appl. No.: 17/601,550

(22) PCT Filed: Apr. 3, 2020
(86) PCT No.: PCT/US20/26503

§ 371 Date: Oct. 5, 2021

Related U.S. Application Data
(60) Provisional application No. 62/829,664, filed on Apr. 5, 2019.

(51) Int. Cl. C12N 15/75; C12N 9/22; C12N 15/11; C12N 15/90; C12N 9/54

(52) U.S. Cl. CPC C12N 15/75; C12N 9/22; C12N 15/11; C12N 15/902; C12N 2800/40; C12Y 304/21014; C12N 2310/20; C12N 2800/80; C12N 2800/101; C12N 9/54

(57) ABSTRACT

Methods and compositions are provided for integrating genes of interest into the genome of a Bacillus sp. cell without the integration of a selectable marker into said genome. The methods employ a dual circular recombinant DNA system for introduction of a guide RNA/Cas endonuclease system (also referred to as an RNA guided endonuclease, RGEN) as well as a donor DNA into a Bacillus sp. cell, and providing a highly effective system for inserting genes of interest into the genome of said Bacillus sp. cell.

### CROSS REFERENCE OF RELATED APPLICATIONS

This application claims the benefit of U.S. Application No. 62/829,664, filed Apr. 5, 2019, and is herein incorporated by reference in its entirety.

### FIELD OF INVENTION

The invention relates to the field of bacterial molecular biology, in particular, to compositions and methods for integrating polynucleotides of interest into a target site on the genome of Bacillus sp. cells

### REFERENCE TO SEQUENCE LISTING SUBMITTED ELECTRONICALLY

The official copy of the sequence listing is submitted electronically via EFS-Web as an ASCII formatted sequence listing with a file named 20200319_NB41332WOPCT_ST25.txt created on Mar. 20, 2020 date, and having a size of 151 kilobytes and is filed concurrently with the specification. The sequence listing contained in this ASCII-formatted document is part of the specification and is herein incorporated by reference in its entirety.

### BACKGROUND

Recombinant DNA technology has made it possible to insert DNA sequences at targeted genomic locations. Site-specific integration techniques, which employ site-specific recombination systems, as well as other types of recombination technologies, have been used to generate targeted insertions of genes of interest in a variety of organism. Given the site-specific nature of Cas systems, genome engineering techniques based on these systems have been described, including in mammalian cells (see, e.g., Hsu et al., 2014). Cas-based genome engineering, when functioning as intended, confers the ability to target virtually any specific location within a complex genome, by designing a recombinant crRNA (or equivalently functional guide RNA) in which the DNA-targeting region (i.e., the variable targeting domain) of the crRNA is homologous to a desired target site in the genome, and combining the crRNA with a Cas endonuclease (through any convenient and conventional means) into a functional complex in a host cell. The sequence of the RNA component of Cas9 can be designed such that Cas9 recognizes and cleaves DNA containing (i) sequence complementary to a portion of the RNA component and (ii) a protospacer adjacent motif (PAM) sequence.

Although Cas-based genome engineering techniques have been applied to a number of different host cell types, these techniques have known limitations

Previous methods for gene integration into the genome of Bacillus sp. cells relied on spontaneous double strand break occurrence and use of selectable markers co-located on linear DNA fragments with short homology arms (comprising both the gene of interest (GOI) to be inserted into the genome as well as a selectable marker that was also inserted into the genome to enable identification of Bacillus sp. cells that had the gene of interest integrated into its genome (WO02/14490, published on Feb. 21, 2002). The selectable marker and GOI were typically flanked by two short homology arms such that upon recombination with the DNA within the cell both the GOI and the selectable marker would be integrated in the DNA of the cell. The use of selectable markers during transformation of such linear fragments with short homology arms for genome integration into Bacillus cells is required to select for efficient modification of a specific locus of the genome. The marker must integrate into the correct locus for expression and this integration relies on rare, spontaneous DNA damage that occurs in a stoichastic manner within the population and within the genome. This rare event can only be selected for by combining the use of a marker and chromosomal integration. (WO02/14490, published on Feb. 21, 2002).

The present disclosure describes a method for generating site specific DNA damage (at a target site in the genome) that essentially converts a majority of the population to cells which containing DNA damage at the desired locus. Hence, this is no longer the limiting step for modifying a chromosomal locus; instead the limiting feature is transformation efficiency and thus the selectable markers are required to differentiate transformed from non-transformed cells.

In Bacillus subtilis, use of a single plasmid system in combination with Cas/RNA guided system in Bacillus subtilis has been described for allowing gene deletions and introduction of point mutations in genes (Altenbuchner J., 2016, Applied and Environmental Microbiology, vol. 82 (17) pg. 5421-5427).

There remains a need for developing effective, efficient or otherwise more robust or flexible Cas-based methods, and compositions thereof, for integrating polynucleotides of interest (such as but not limiting to a gene of interest, a single copy gene expression cassette or multi-copy gene expression cassette) into a target site on the genome of a Bacillus sp. cell.

### BRIEF SUMMARY

The present disclosure includes methods and compositions for integrating polynucleotides of interest into the genome of a Bacillus sp. cell without the need to integrate a selectable marker into said genome. The methods employ a dual circular recombinant DNA system for introduction of a guide RNA/Cas endonuclease system (also referred to as an RNA guided endonuclease, RGEN) as well as a donor DNA (comprising the polynucleotide of interest) into a Bacillus sp. cell, and providing a highly effective system for integrating polynucleotides of interest into the genome of said Bacillus sp. cell, without the need to integrate a selectable marker in the genome of said Bacillus sp. cell.

In one aspect, the method described herein comprises a method for integrating a gene of interest into a target site on the genome of a Bacillus sp. cell without the integration of a selectable marker, the method comprising simultaneously introducing at least a first circular recombinant DNA construct and a second circular recombinant DNA construct into a Bacillus sp. cell, wherein said first circular recombinant DNA construct comprises a donor DNA sequence comprising a gene of interest and a DNA sequence encoding a guide RNA, wherein said second circular recombinant DNA construct comprises a Cas9 endonuclease DNA sequence operably linked to a constitutive promoter, wherein said Cas9 endonuclease DNA sequence encodes a Cas9 that introduces a double-strand break at or near a target site in the genome of said Bacillus sp. cell. The donor DNA sequence is flanked by two homology arms, one upstream arm (5′ HR1) and one downstream arm (3′ HR2) wherein both homology arms (HR1 and HR2) are equal to about 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, or up to 600 nucleotides in length, or up to 600 nucleotides in length and comprise sequence homology to a targeted genomic locus of said Bacillus sp. cell.

In one aspect, the first and/or second circular recombinant DNA construct comprise a selectable marker that is used to facilitate selection of transformed Bacillus sp. cells, but is not necessary for selection of (daughter) Bacillus sp. cells that have the gene of interest integrated into its genome. These daughter Bacillus sp. cells have lost the first and second circular recombinant DNA construct comprising the selectable maker, and as such have no selectable marker integrated into their genome (FIG. 1). As such, the method can further comprise growing progeny cells from said Bacillus sp. cell and selecting a Bacillus sp. progeny cell that does not contain the first and/or second circular recombinant DNA construct (and does not contain the selectable marker comprised on these circular recombinant DNAs) but has the gene of interest stably integrated in its genome.

In some embodiments, the method described above results in a frequency of integration of the gene of interest gene into the genome of the Bacillus sp. cell that is at least about 2, 3, 4, 5, 6, 7, 8, 9, 10 up to 11 fold higher when compared to the frequency of integration of a control method comprising introducing into a Bacillus sp. cell a linear recombinant DNA construct comprising said donor DNA sequence flanked by an upstream (5′ HR1) and downstream homology arm (3′ HR2) of 1000 bps, and a circular recombinant DNA construct comprising an expression cassette for Cas9 and an expression cassette for gRNA. (FIG. 2.)

In some embodiments, the Bacillus sp. cell is selected from the group consisting of Bacillus subtilis, Bacillus licheniformis, Bacillus lentus, Bacillus brevis, Bacillus stearothermophilus, Bacillus alkalophilus, Bacillus amyloliquefaciens, Bacillus clausii, Bacillus. halodurans, Bacillus. megaterium, Bacillus coagulans, Bacillus circulans, Bacillus lautus, and Bacillus thuringiensis.

In one embodiment, the disclosure concerns a Bacillus sp. cell comprising at least a first circular recombinant DNA construct and a second circular recombinant DNA construct, wherein said first circular recombinant DNA construct comprises a DNA sequence encoding a guide RNA and comprises a donor DNA sequence comprising a gene of interest encoding a protein of interest, wherein said guide RNA comprises a sequence complementary to a target site sequence on a chromosome or episome of said Bacillus sp. cell, wherein said second circular recombinant DNA construct comprises a Cas9 endonuclease DNA sequence operably linked to a constitutive promoter, wherein said Cas9 endonuclease DNA sequence encodes a Cas9 endonuclease that can form a RNA-guided endonuclease (RGEN), wherein said RGEN can bind to, and optionally cleave, all or part of the target site sequence.

### BRIEF DESCRIPTION OF THE DRAWINGS AND SEQUENCES

FIG. 1 depicts the integration of a gene of interest into the Bacillus sp. genome using the dual recombinant DNA construct system described herein, said system comprising two circular recombinant DNA constructs that are simultaneously introduced into the Bacillus sp. cell. In this illustration, the first circular recombinant DNA comprises a donor DNA comprising a gene of interest (GOI), wherein the donor DNA is flanked by two homology arms (one 5′ upstream arm, HR1, and one 3′ downstream arm HR2) wherein each homology arm is equal to about 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, or up to 600 nucleotides in length and comprises sequence homology to a targeted genomic locus of said Bacillus sp. cell., as well as a DNA sequence encoding a guide RNA, wherein the second circular recombinant DNA comprises a DNA sequence (encoding a Cas9 endonuclease) operably linked to a constitutive promoter. Applicants have surprisingly found that when such a dual recombinant DNA system is used to insert a GOI into the Bacillus genome without the integration of a selectable marker into said genome, a significant increase in frequency of gene insertion (up to 11 fold) is observed, when compared to the frequency of gene insertion of a control method (such as the control method depicted in FIG. 2).

FIG. 2. depicts the integration of a gene of interest into the Bacillus sp. genome using a control method described herein, said method comprising a (first) linear recombinant DNA and a (second) circular recombinant DNA that are simultaneously introduced into the Bacillus sp. cell. In this illustration, the linear recombinant DNA comprises a donor DNA comprising a gene of interest, wherein the donor is flanked by two homology arms, one upstream arm (5′ HR1) and one downstream arm (3′ HR2) wherein each homology arm is 1000 nucleotides in length and comprises sequence homology to a targeted genomic locus of said Bacillus sp. cell.

### DETAILED DESCRIPTION

The present disclosure includes methods and compositions for integrating genes of interest into the genome of a Bacillus sp. cell without the integration of a selectable marker into said genome. The methods employ a dual circular recombinant DNA system for introduction of a guide RNA/Cas endonuclease system (also referred to as an RNA guided endonuclease, RGEN) as well as a donor DNA into a Bacillus sp. cell, and providing a highly effective system for inserting genes of interest into the genome of said Bacillus sp. cell.

The present document is organized into a number of sections for ease of reading; however, the reader will appreciate that statements made in one section may apply to other sections. In this manner, the headings used for different sections of the disclosure should not be construed as limiting.

The headings provided herein are not limitations of the various aspects or embodiments of the present compositions and methods which can be had by reference to the specification as a whole. Accordingly, the terms defined immediately below are more fully defined by reference to the specification as a whole.

Unless defined otherwise, all technical and scientific terms used herein have the same meaning as commonly understood by one of ordinary skill in the art to which the present compositions and methods belongs. Although any methods and materials similar or equivalent to those described herein can also be used in the practice or testing of the present compositions and methods, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited.

As used herein, the term “disclosure” or “disclosed disclosure” is not meant to be limiting, but applies generally to any of the disclosures defined in the claims or described herein. These terms are used interchangeably herein.

### Cas Genes and Proteins

CRISPR (clustered regularly interspaced short palindromic repeats) loci refers to certain genetic loci encoding components of DNA cleavage systems, for example, used by bacterial and archaeal cells to destroy foreign DNA (Horvath and Barrangou, 2010, Science 327:167-170; WO2007/025097, published Mar. 1, 2007). A CRISPR locus can consist of a CRISPR array, comprising short direct repeats (CRISPR repeats) separated by short variable DNA sequences (called ‘spacers’), which can be flanked by diverse Cas (CRISPR-associated) genes. The number of CRISPR-associated genes at a given CRISPR locus can vary between species. Multiple CRISPR/Cas systems have been described including Class 1 systems, with multisubunit effector complexes (comprising type I, type III and type IV subtypes), and Class 2 systems, with single protein effectors (comprising type II and type V subtypes, such as but not limiting to Cas9, Cpf1, C2c1, C2c2, C2c3). Class 1 systems (Makarova et al. 2015, Nature Reviews; Microbiology Vol. 13:1-15; Zetsche et al., 2015, Cell 163, 1-13; Shmakov et al., 2015, Molecular_Cell 60, 1-13; Haft et al., 2005, Computational Biology, PLoS Comput Biol 1(6): e60. doi:10.1371/journal.pcbi. 0010060 and WO 2013/176772 A1 published on Nov. 23, 2013 incorporated by reference herein). The type II CRISPR/Cas system from bacteria employs a crRNA (CRISPR RNA) and tracrRNA (trans-activating CRISPR RNA) to guide the Cas endonuclease to its DNA target. The crRNA contains a spacer region complementary to one strand of the double strand DNA target and a region that base pairs with the tracrRNA (trans-activating CRISPR RNA) forming a RNA duplex that directs the Cas endonuclease to cleave the DNA target. Spacers are acquired through a not fully understood process involving Cas1 and Cas2 proteins. All type II CRISPR/Cas loci contain cas1 and cas2 genes in addition to the cas9 gene (Chylinski et al., 2013, RNA Biology 10:726-737; Makarova et al. 2015, Nature Reviews Microbiology Vol. 13:1-15). Type II CRISPR-Cas loci can encode a tracrRNA, which is partially complementary to the repeats within the respective CRISPR array, and can comprise other proteins such as Csn1 and Csn2. The presence of cas9 in the vicinity of Cas 1 and cas2 genes is the hallmark of type II loci (Makarova et al. 2015, Nature Reviews Microbiology Vol. 13:1-15). Type I CRISPR-Cas (CRISPR-associated) systems consist of a complex of proteins, termed Cascade (CRISPR-associated complex for antiviral defense), which function together with a single CRISPR RNA (crRNA) and Cas3 to defend against invading viral DNA (Brouns, S. J. J. et al. Science 321:960-964; Makarova et al. 2015, Nature Reviews; Microbiology Vol. 13:1-15, which are incorporated in their entirety herein).

The term “Cas gene” herein refers to a gene that is generally coupled, associated or close to, or in the vicinity of flanking CRISPR loci. The terms “Cas gene”, “cas gene”, “CRISPR-associated (Cas) gene” and “Clustered Regularly Interspaced Short Palindromic Repeats-associated gene” are used interchangeably herein.

The term “Cas protein” or “Cas polypeptide” refers to a polypeptide encoded by a Cas (CRISPR-associated) gene. A Cas protein includes a Cas endonuclease.

A Cas protein may be a bacterial or archaeal protein. Type I-III CRISPR Cas proteins herein are typically prokaryotic in origin; type I and III Cas proteins can be derived from bacterial or archaeal species, whereas type II Cas proteins (i.e., a Cas9) can be derived from bacterial species, for example. In other aspects, Cas proteins include one or more of Cas1, Cas1B, Cas2, Cas3, Cas4, Cas5, Cas6, Cas7, Cas8, Cas9, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. A Cas protein includes a Cas9 protein, a Cpf1 protein, a C2c1 protein, a C2c2 protein, a C2c3 protein, Cas3, Cas3-HD, Cas 5, Cas7, Cas8, Cas10, or combinations or complexes of these.

The term “Cas endonuclease” refers to a Cas polypeptide (Cas protein) that, when in complex with a suitable polynucleotide component, is capable of recognizing, binding to, and optionally nicking or cleaving all or part of a specific DNA target sequence. A Cas endonuclease is guided by the guide polynucleotide to recognize, bind to, and optionally nick or cleave all or part of a specific target site in double stranded DNA (e.g., at a target site in the genome of a cell). A Cas endonuclease described herein comprises one or more nuclease domains. The Cas endonucleases employed in donor DNA insertion methods described herein are endonucleases that introduce single or double-strand breaks into the DNA at the target site. Alternatively, a Cas endonuclease may lack DNA cleavage or nicking activity, but can still specifically bind to a DNA target sequence when complexed with a suitable RNA component.

As used herein, a polypeptide referred to as a “Cas9” (formerly referred to as Cas5, Csn1, or Csx12) or a “Cas9 endonuclease” or having “Cas9 endonuclease activity” refers to a Cas endonuclease that forms a complex with a crNucleotide and a tracrNucleotide, or with a single guide polynucleotide, for specifically binding to, and optionally nicking or cleaving all or part of a DNA target sequence. A Cas9 endonuclease comprises a RuvC nuclease domain and an HNH (H—N—H) nuclease domain, each of which can cleave a single DNA strand at a target sequence (the concerted action of both domains leads to DNA double-strand cleavage, whereas activity of one domain leads to a nick). In general, the RuvC domain comprises subdomains I, II and III, where domain I is located near the N-terminus of Cas9 and subdomains II and III are located in the middle of the protein, flanking the HNH domain (Makarova et al. 2015, Nature Reviews Microbiology Vol. 13:1-15, Hsu et al, 2013, Cell 157:1262-1278). Cas9 endonucleases are typically derived from a type II CRISPR system, which includes a DNA cleavage system utilizing a Cas9 endonuclease in complex with at least one polynucleotide component. For example, a Cas9 can be in complex with a CRISPR RNA (crRNA) and a trans-activating CRISPR RNA (tracrRNA). In another example, a Cas9 can be in complex with a single guide RNA (Makarova et al. 2015, Nature Reviews Microbiology Vol. 13:1-15).

A “functional fragment”, “fragment that is functionally equivalent” and “functionally equivalent fragment” of a Cas endonuclease are used interchangeably herein, and refer to a portion or subsequence of the Cas endonuclease in which the ability to recognize, bind to, and optionally unwind, nick or cleave (introduce a single or double-strand break in) the target site is retained.

The terms “functional variant”, “variant that is functionally equivalent” and “functionally equivalent variant” of a Cas endonuclease of the present disclosure, are used interchangeably herein, and refer to a variant of the Cas endonuclease of the present disclosure in which the ability to recognize, bind to, and optionally unwind, nick or cleave all or part of a target sequence is retained.

Determining binding activity and/or endonucleolytic activity of a Cas protein herein toward a specific target DNA sequence may be assessed by any suitable assay known in the art, such as disclosed in U.S. Pat. No. 8,697,359, which is disclosed herein by reference. A determination can be made, for example, by expressing a Cas protein and suitable RNA component in host cell/organism, and then examining the predicted DNA target site for the presence of an indel (a Cas protein in this particular assay would have endonucleolytic activity [single or double-strand cleaving activity]). Examining for the presence of an indel at the predicted target site could be done via a DNA sequencing method or by inferring indel formation by assaying for loss of function of the target sequence, for example. In another example, Cas protein activity can be determined by expressing a Cas protein and suitable RNA component in a host cell/organism that has been provided a donor DNA comprising a sequence homologous to a sequence in at or near the target site. The presence of donor DNA sequence at the target site (such as would be predicted by successful HR between the donor and target sequences) would indicate that targeting occurred.

Non limiting examples of Cas endonucleases herein can be Cas endonucleases from any of the following genera: Aeropyrum, Pyrobaculum, Sulfolobus, Archaeoglobus, Haloarcula, Methanobacteriumn, Methanococcus, Methanosarcina, Methanopyrus, Pyrococcus, Picrophilus, Themioplasnia, Corynebacterium, Mycobacterium, Streptomyces, Aquifrx, Porphvromonas, Chlorobium, Thermus, Bacillus, Listeria, Staphylococcus, Clostridium, Thermoanaerobacter, Mycoplasma, Fusobacterium, Azarcus, Chromobacterium, Neisseria, Nitrosomonas, Desulfovibrio, Geobacter, Myrococcus, Campylobacter, Wolinella, Acinetobacter, Erwinia, Escherichia, Legionella, Methylococcus, Pasteurella, Photobacterium, Salmonella, Xanthomonas, Yersinia, Streptococcus, Treponema, Francisella, or Thermotoga. Furthermore, a Cas endonuclease herein can be encoded, for example, by any of SEQ ID NOs:462-465, 467-472, 474-477, 479-487, 489-492, 494-497, 499-503, 505-508, 510-516, or 517-521 as disclosed in U.S. Appl. Publ. No. 2010/0093617, which is incorporated herein by reference.

Furthermore, a Cas9 endonuclease herein may be derived from a Streptococcus (e.g., S. pyogenes, S. pneumoniae, S. thermophilus, S. agalactiae, S. parasanguinis, S. oralis, S. salivarius, S. macacae, S. dysgalactiae, S. anginosus, S. constellatus, S. pseudoporcinus, S. mutans), Listeria (e.g., L. innocua), Spiroplasma (e.g., S. apis, S. syrphidicola), Peptostreptococcaceae, Atopobium, Porphyromonas (e.g., P. catoniae), Prevotella (e.g., P. intermedia), Veillonella, Treponema (e.g., T. socranskii, T. denticola), Capnocytophaga, Finegoldia (e.g., F. magna), Coriobacteriaceae (e.g., C. bacterium), Olsenella (e.g., O. profusa), Haemophilus (e.g., H. sputorum, H. pittmaniae), Pasteurella (e.g., P. bettyae), Olivibacter (e.g., O. sitiensis), Epilithonimonas (e.g., E. tenax), Mesonia (e.g., M. mobilis), Lactobacillus (e.g., L. plantarum), Bacillus (e.g., B. cereus), Aquimarina (e.g., A. muelleri), Chryseobacterium (e.g., C. palustre), Bacteroides (e.g., B. graminisolvens), Neisseria (e.g., N. meningitidis), Francisella (e.g., F. novicida), or Flavobacterium (e.g., F. frigidarium, F. soli) species, for example. In one aspect a S. pyogenes Cas9 endonuclease is described herein. As another example, a Cas9 endonuclease can be any of the Cas9 proteins disclosed in Chylinski et al. (RNA Biology 10:726-737), which is incorporated herein by reference.

The sequence of a Cas9 endonuclease herein can comprise, for example, any of the Cas9 amino acid sequences disclosed in GenBank Accession Nos. G3ECR1 (S. thermophilus), WP_026709422, WP_027202655, WP_027318179, WP_027347504, WP_027376815, WP_027414302, WP_027821588, WP_027886314, WP_027963583, WP_028123848, WP_028298935, Q03JI6 (S. thermophilus), EGP66723, EGS38969, EGV05092, EHI65578 (S. pseudoporcinus), EIC75614 (S. oralis), EID22027 (S. constellatus), EIJ69711, EJP22331 (S. oralis), EJP26004 (S. anginosus), EJP30321, EPZ44001 (S. pyogenes), EPZ46028 (S. pyogenes), EQL78043 (S. pyogenes), EQL78548 (S. pyogenes), ERL10511, ERL12345, ERL19088 (S. pyogenes), ESA57807 (S. pyogenes), ESA59254 (S. pyogenes), ESU85303 (S. pyogenes), ETS96804, UC75522, EGR87316 (S. dysgalactiae), EGS33732, EGV01468 (S. oralis), EHJ52063 (S. macacae), EID26207 (S. oralis), EID33364, EIG27013 (S. parasanguinis), EJF37476, EJO19166 (Streptococcus sp. BS35b), EJU16049, EJU32481, YP_006298249, ERF61304, ERK04546, ETJ95568 (S. agalactiae), TS89875, ETS90967 (Streptococcus sp. SR4), ETS92439, EUB27844 (Streptococcus sp. BS21), AFJ08616, EUC82735 (Streptococcus sp. CM6), EWC92088, EWC94390, EJP25691, YP_008027038, YP_008868573, AGM26527, AHK22391, AHB36273, Q927P4, G3ECR1, or Q99ZW2 (S. pyogenes), which are incorporated by reference. Alternatively, a Cas9 protein herein can be encoded by any of SEQ ID NOs:462 (S. thermophilus), 474 (S. thermophilus), 489 (S. agalactiae), 494 (S. agalactiae), 499 (S. mutans), 505 (S. pyogenes), or 518 (S. pyogenes) as disclosed in U.S. Appl. Publ. No. 2010/0093617 (incorporated herein by reference), for example.

Given that certain amino acids share similar structural and/or charge features with each other (i.e., conserved), the amino acid at each position in a Cas9 can be as provided in the disclosed sequences or substituted with a conserved amino acid residue (“conservative amino acid substitution”) as follows:
1. The following small aliphatic, nonpolar or slightly polar residues can substitute for each other: Ala (A), Ser (S), Thr (T), Pro (P), Gly (G);
2. The following polar, negatively charged residues and their amides can substitute for each other: Asp (D), Asn (N), Glu (E), Gln (Q);
3. The following polar, positively charged residues can substitute for each other: His (H), Arg (R), Lys (K);
4. The following aliphatic, nonpolar residues can substitute for each other: Ala (A), Leu (L), Ile (I), Val (V), Cys (C), Met (M); and
5. The following large aromatic residues can substitute for each other: Phe (F), Tyr (Y), Trp (W).

Fragments and variants can be obtained via methods such as site-directed mutagenesis and synthetic construction. Methods for measuring endonuclease activity are well known in the art such as, but not limiting to, PCT/US13/39011, filed May 1, 2013, PCT/US16/32073 filed May 12, 2016, PCT/US16/32028 filed May 12, 2016, incorporated by reference herein).

The Cas endonuclease can comprise a modified form of the Cas polypeptide. The modified form of the Cas polypeptide can include an amino acid change (e.g., deletion, insertion, or substitution) that reduces the naturally-occurring nuclease activity of the Cas protein. For example, in some instances, the modified form of the Cas protein has less than 50%, less than 40%, less than 30%, less than 20%, less than 10%, less than 5%, or less than 1% of the nuclease activity of the corresponding wild-type Cas polypeptide (US patent application US20140068797 A1, published on Mar. 6, 2014). In some cases, the modified form of the Cas polypeptide has no substantial nuclease activity and is referred to as catalytically “inactivated Cas” or “deactivated Cas (dCas).” An inactivated Cas/deactivated Cas includes a deactivated Cas endonuclease (dCas). A catalytically inactive Cas can be fused to a heterologous sequence. Other Cas9 variants lack the activity of either the HNH or the RuvC nuclease domains and are thus proficient to cleave only 1 strand of the DNA (nickase variants).

Recombinant DNA constructs expressing the Cas endonuclease described herein can be transiently integrated into a Bacillus sp. cell or stably integrated into the genome of a Bacillus sp. cell.

Cas Protein Fusions

A Cas endonuclease can be part of a fusion protein comprising one or more heterologous protein domains (e.g., 1, 2, 3, or more domains in addition to the Cas polypeptide). Such a fusion protein may comprise any additional protein sequence, and optionally a linker sequence between any two domains, such as between Cas polypeptide and a first heterologous domain. Examples of protein domains that may be fused to a Cas polypeptide include, without limitation, epitope tags (e.g., histidine [His], V5, FLAG, influenza hemagglutinin [HA], myc, VSV-G, thioredoxin [Trx]), reporters (e.g., glutathione-5-transferase [GST], horseradish peroxidase [HRP], chloramphenicol acetyltransferase [CAT], beta-galactosidase, beta-glucuronidase [GUS], luciferase, green fluorescent protein [GFP], HcRed, DsRed, cyan fluorescent protein [CFP], yellow fluorescent protein [YFP], blue fluorescent protein [BFP]), and domains having one or more of the following activities: methylase activity, demethylase activity, transcription activation activity (e.g., VP16 or VP64), transcription repression activity, transcription release factor activity, histone modification activity, RNA cleavage activity and nucleic acid binding activity. A Cas endonuclease can also be in fusion with a protein that binds DNA molecules or other molecules, such as maltose binding protein (MBP), S-tag, Lex A DNA binding domain (DBD), GAL4A DNA binding domain, and herpes simplex virus (HSV) VP16.

A Cas endonuclease can comprise a heterologous regulatory element such as a nuclear localization sequence (NLS). A heterologous NLS amino acid sequence may be of sufficient strength to drive accumulation of a Cas endonuclease in a detectable amount in the nucleus of a cell herein. An NLS may comprise one (monopartite) or more (e.g., bipartite) short sequences (e.g., 2 to 20 residues) of basic, positively charged residues (e.g., lysine and/or arginine), and can be located anywhere in a Cas amino acid sequence but such that it is exposed on the protein surface. An NLS may be operably linked to the N-terminus or C-terminus of a Cas protein herein, for example. Two or more NLS sequences can be linked to a Cas protein, for example, such as on both the N- and C-termini of a Cas protein. The Cas gene can be operably linked to a SV40 nuclear targeting signal upstream of the Cas codon region and a bipartite VirD2 nuclear localization signal (Tinland et al. (1992) Proc. Natl. Acad. Sci. USA 89:7442-6) downstream of the Cas codon region. Non-limiting examples of suitable NLS sequences herein include those disclosed in U.S. Pat. Nos. 6,660,830 and 7,309,576, which are both incorporated by reference herein. A heterologous NLS amino acid sequence include plant, viral and mammalian nuclear localization signals.

A catalytically active and/or inactive Cas endonuclease, can be fused to a heterologous sequence (US patent application US20140068797 A1, published on Mar. 6, 2014). Suitable fusion partners include, but are not limited to, a polypeptide that provides an activity that indirectly increases transcription by acting directly on the target DNA or on a polypeptide (e.g., a histone or other DNA-binding protein) associated with the target DNA. Additional suitable fusion partners include, but are not limited to, a polypeptide that provides for methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, or demyristoylation activity. Further suitable fusion partners include, but are not limited to, a polypeptide that directly provides for increased transcription of the target nucleic acid (e.g., a transcription activator or a fragment thereof, a protein or fragment thereof that recruits a transcription activator, a small molecule/drug-responsive transcription regulator, etc.). A catalytically inactive Cas9 endonuclease can also be fused to a FokI nuclease to generate double-strand breaks (Guilinger et al. Nature biotechnology, volume 32, number 6, June 2014).

### Guide Polynucleotide, Guide RNA

As used herein, the term “guide polynucleotide”, relates to a polynucleotide sequence that can form a complex with a Cas endonuclease, and enables the Cas endonuclease to recognize, bind to, and optionally nick or cleave a DNA target site. The guide polynucleotide can be a single molecule or a double molecule. The guide polynucleotide sequence can be a RNA sequence, a DNA sequence, or a combination thereof (a RNA-DNA combination sequence). Optionally, the guide polynucleotide can comprise at least one nucleotide, phosphodiester bond or linkage modification such as, but not limited, to Locked Nucleic Acid (LNA), 5-methyl dC, 2,6-Diaminopurine, 2′-Fluoro A, 2′-Fluoro U, 2′-O-Methyl RNA, phosphorothioate bond, linkage to a cholesterol molecule, linkage to a polyethylene glycol molecule, linkage to a spacer 18 (hexaethylene glycol chain) molecule, or 5′ to 3′ covalent linkage resulting in circularization. A guide polynucleotide that solely comprises ribonucleic acids is also referred to as a “guide RNA” or “gRNA”.

The guide polynucleotide can be a double molecule (also referred to as duplex guide polynucleotide) comprising a crNucleotide sequence and a tracrNucleotide sequence. The crNucleotide includes a first nucleotide sequence domain (referred to as Variable Targeting domain or VT domain) that can hybridize to a nucleotide sequence in a target DNA and a second nucleotide sequence (also referred to as a tracr mate sequence) that is part of a Cas endonuclease recognition (CER) domain. The tracr mate sequence can hybridized to a tracrNucleotide along a region of complementarity and together form the Cas endonuclease recognition domain or CER domain. The CER domain is capable of interacting with a Cas endonuclease polypeptide. The crNucleotide and the tracrNucleotide of the duplex guide polynucleotide can be RNA, DNA, and/or RNA-DNA-combination sequences. (U.S. Patent Application US20150082478, published on Mar. 19, 2015 and US20150059010, published on Feb. 26, 2015, both are herein incorporated by reference). In some embodiments, the crNucleotide molecule of the duplex guide polynucleotide is referred to as “crDNA” (when composed of a contiguous stretch of DNA nucleotides) or “crRNA” (when composed of a contiguous stretch of RNA nucleotides), or “crDNA-RNA” (when composed of a combination of DNA and RNA nucleotides). The crNucleotide can comprise a fragment of the crRNA naturally occurring in Bacteria and Archaea. The size of the fragment of the crRNA naturally occurring in Bacteria and Archaea that can be present in a crNucleotide disclosed herein can range from, but is not limited to, 2, 3, 4, 5, 6, 7, 8, 9, 10, 11, 12, 13, 14, 15, 16, 17, 18, 19, 20 or more nucleotides. In some embodiments the tracrNucleotide is referred to as “tracrRNA” (when composed of a contiguous stretch of RNA nucleotides) or “tracrDNA” (when composed of a contiguous stretch of DNA nucleotides) or “tracrDNA-RNA” (when composed of a combination of DNA and RNA nucleotides. In certain embodiments, the RNA that guides the RNA/Cas9 endonuclease complex is a duplexed RNA comprising a duplex crRNA-tracrRNA.

The guide polynucleotide includes a dual RNA molecule comprising a chimeric non-naturally occurring crRNA (non-covalently) linked to at least one tracrRNA. A chimeric non-naturally occurring crRNA includes a crRNA that comprises regions that are not found together in nature (i.e., they are heterologous with each other). For example, a non-naturally occurring crRNA is a crRNA wherein the naturally occurring spacer sequence is exchanged for a heterologous Variable Targeting domain. A non-naturally occurring crRNA comprises a first nucleotide sequence domain (referred to as Variable Targeting domain or VT domain) that can hybridize to a nucleotide sequence in a target DNA, linked to a second nucleotide sequence (also referred to as a tracr mate sequence) such that the first and second sequence are not found linked together in nature.

The guide polynucleotide can also be a single molecule (also referred to as single guide polynucleotide) comprising a crNucleotide sequence linked to a tracrNucleotide sequence. The single guide polynucleotide comprises a first nucleotide sequence domain (referred to as Variable Targeting domain or VT domain) that can hybridize to a nucleotide sequence in a target DNA and a Cas endonuclease recognition domain (CER domain), that interacts with a Cas endonuclease polypeptide. By “domain” it is meant a contiguous stretch of nucleotides that can be RNA, DNA, and/or RNA-DNA-combination sequence. The VT domain and/or the CER domain of a single guide polynucleotide can comprise a RNA sequence, a DNA sequence, or a RNA-DNA-combination sequence. The single guide polynucleotide being comprised of sequences from the crNucleotide and the tracrNucleotide may be referred to as “single guide RNA” (when composed of a contiguous stretch of RNA nucleotides) or “single guide DNA” (when composed of a contiguous stretch of DNA nucleotides) or “single guide RNA-DNA” (when composed of a combination of RNA and DNA nucleotides). The single guide polynucleotide can form a complex with a Cas endonuclease, wherein said guide polynucleotide/Cas endonuclease complex (also referred to as a guide polynucleotide/Cas endonuclease system) can direct the Cas endonuclease to a genomic target site, enabling the Cas endonuclease to recognize, bind to, and optionally nick or cleave (introduce a single or double-strand break) the target site.

The term “variable targeting domain” or “VT domain” is used interchangeably herein and includes a nucleotide sequence that can hybridize (is complementary) to one strand (nucleotide sequence) of a double strand DNA target site. The % complementation between the first nucleotide sequence domain (VT domain) and the target sequence can be at least 50%, 51%, 52%, 53%, 54%, 55%, 56%, 57%, 58%, 59%, 60%, 61%, 62%, 63%, 63%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. The variable targeting domain can be at least 12, 13, 14, 15, 16, 17, 18, 19, 20, 21, 22, 23, 24, 25, 26, 27, 28, 29 or 30 nucleotides in length.

The variable targeting domain can comprises a contiguous stretch of 12 to 30, 12 to 29, 12 to 28, 12 to 27, 12 to 26, 12 to 25, 12 to 26, 12 to 25, 12 to 24, 12 to 23, 12 to 22, 12 to 21, 12 to 20, 12 to 19, 12 to 18, 12 to 17, 12 to 16, 12 to 15, 12 to 14, 12 to 13, 13 to 30, 13 to 29, 13 to 28, 13 to 27, 13 to 26, 13 to 25, 13 to 26, 13 to 25, 13 to 24, 13 to 23, 13 to 22, 13 to 21, 13 to 20, 13 to 19, 13 to 18, 13 to 17, 13 to 16, 13 to 15, 13 to 14, 14 to 30, 14 to 29, 14 to 28, 14 to 27, 14 to 26, 14 to 25, 14 to 26, 14 to 25, 14 to 24, 14 to 23, 14 to 22, 14 to 21, 14 to 20, 14 to 19, 14 to 18, 14 to 17, 14 to 16, 14 to 15, 15 to 30, 15 to 29, 15 to 28, 15 to 27, 15 to 26, 15 to 25, 15 to 26, 15 to 25, 15 to 24, 15 to 23, 15 to 22, 15 to 21, 15 to 20, 15 to 19, 15 to 18, 15 to 17, 15 to 16, 16 to 30, 16 to 29, 16 to 28, 16 to 27, 16 to 26, 16 to 25, 16 to 24, 16 to 23, 16 to 22, 16 to 21, 16 to 20, 16 to 19, 16 to 18, 16 to 17, 17 to 30, 17 to 29, 17 to 28, 17 to 27, 17 to 26, 17 to 25, 17 to 24, 17 to 23, 17 to 22, 17 to 21, 17 to 20, 17 to 19, 17 to 18, 18 to 30, 18 to 29, 18 to 28, 18 to 27, 18 to 26, 18 to 25, 18 to 24, 18 to 23, 18 to 22, 18 to 21, 18 to 20, 18 to 19, 19 to 30, 19 to 29, 19 to 28, 19 to 27, 19 to 26, 19 to 25, 19 to 24, 19 to 23, 19 to 22, 19 to 21, 19 to 20, 20 to 30, 20 to 29, 20 to 28, 20 to 27, 20 to 26, 20 to 25, 20 to 24, 20 to 23, 20 to 22, 20 to 21, 21 to 30, 21 to 29, 21 to 28, 21 to 27, 21 to 26, 21 to 25, 21 to 24, 21 to 23, 21 to 22, 22 to 30, 22 to 29, 22 to 28, 22 to 27, 22 to 26, 22 to 25, 22 to 24, 22 to 23, 23 to 30, 23 to 29, 23 to 28, 23 to 27, 23 to 26, 23 to 25, 23 to 24, 24 to 30, 24 to 29, 24 to 28, 24 to 27, 24 to 26, 24 to 25, 25 to 30, 25 to 29, 25 to 28, 25 to 27, 25 to 26, 26 to 30, 26 to 29, 26 to 28, 26 to 27, 27 to 30, 27 to 29, 27 to 28, 28 to 30, 28 to 29, or 29 to 30 nucleotides.

The variable targeting domain can be composed of a DNA sequence, a RNA sequence, a modified DNA sequence, a modified RNA sequence, or any combination thereof. The VT domain can be complementary to target sequences derived from prokaryotic or eukaryotic DNA.

The term “Cas endonuclease recognition domain” or “CER domain” (of a guide polynucleotide) is used interchangeably herein and includes a nucleotide sequence that interacts with a Cas endonuclease polypeptide. A CER domain comprises a tracrNucleotide mate sequence followed by a tracrNucleotide sequence. The CER domain can be composed of a DNA sequence, a RNA sequence, a modified DNA sequence, a modified RNA sequence (see for example US 2015-0059010 A1, published on Feb. 26, 2015, incorporated in its entirety by reference herein), or any combination thereof.

The nucleotide sequence linking the crNucleotide and the tracrNucleotide of a single guide polynucleotide can comprise a RNA sequence, a DNA sequence, or a RNA-DNA combination sequence. In one embodiment, the nucleotide sequence linking the crNucleotide and the tracrNucleotide of a single guide polynucleotide (also referred to as “loop”) can be at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 78, 79, 80, 81, 82, 83, 84, 85, 86, 87, 88, 89, 90, 91, 92, 93, 94, 95, 96, 97, 98, 99 or 100 nucleotides in length. The loop can be 3-4, 3-5, 3-6, 3-7, 3-8, 3-9, 3-10, 3-11, 3-12, 3-13, 3-14, 3-15, 3-20, 3-30, 3-40, 3-50, 3-60, 3-70, 3-80, 3-90, 3-100, 4-5, 4-6, 4-7, 4-8, 4-9, 4-10, 4-11, 4-12, 4-13, 4-14, 4-15, 4-20, 4-30, 4-40, 4-50, 4-60, 4-70, 4-80, 4-90, 4-100, 5-6, 5-7, 5-8, 5-9, 5-10, 5-11, 5-12, 5-13, 5-14, 5-15, 5-20, 5-30, 5-40, 5-50, 5-60, 5-70, 5-80, 5-90, 5-100, 6-7, 6-8, 6-9, 6-10, 6-11, 6-12, 6-13, 6-14, 6-15, 6-20, 6-30, 6-40, 6-50, 6-60, 6-70, 6-80, 6-90, 6-100, 7-8, 7-9, 7-10, 7-11, 7-12, 7-13, 7-14, 7-15, 7-20, 7-30, 7-40, 7-50, 7-60, 7-70, 7-80, 7-90, 7-100, 8-9, 8-10, 8-11, 8-12, 8-13, 8-14, 8-15, 8-20, 8-30, 8-40, 8-50, 8-60, 8-70, 8-80, 8-90, 8-100, 9-10, 9-11, 9-12, 9-13, 9-14, 9-15, 9-20, 9-30, 9-40, 9-50, 9-60, 9-70, 9-80, 9-90, 9-100, 10-20, 20-30, 30-40, 40-50, 50-60, 70-80, 80-90 or 90-100 nucleotides in length.

In another aspect, the nucleotide sequence linking the crNucleotide and the tracrNucleotide of a single guide polynucleotide can comprise a tetraloop sequence, such as, but not limiting to a GAAA tetraloop sequence.

The single guide polynucleotide includes a chimeric non-naturally occurring single guide RNA. The terms “single guide RNA” and “sgRNA” are used interchangeably herein and relate to a synthetic fusion of two RNA molecules, a crRNA (CRISPR RNA) comprising a variable targeting domain (linked to a tracr mate sequence that hybridizes to a tracrRNA), fused to a tracrRNA (trans-activating CRISPR RNA). A chimeric non-naturally occurring guide RNA comprising regions that are not found together in nature (i.e., they are heterologous with each other). For example, a chimeric non-naturally occurring guide RNA comprising a first nucleotide sequence domain (referred to as Variable Targeting domain or VT domain) that can hybridize to a nucleotide sequence in a target DNA, linked to a second nucleotide sequence that can recognize the Cas endonuclease, such that the first and second nucleotide sequence are not found linked together in nature.

The chimeric non-naturally occurring guide RNA can comprise a crRNA or and a tracrRNA of the type II CRISPR/Cas system that can form a complex with a type II Cas endonuclease, wherein said guide RNA/Cas endonuclease complex can direct the Cas endonuclease to a DNA target site, enabling the Cas endonuclease to recognize, bind to, and optionally nick or cleave (introduce a single or double-strand break) the DNA target site.

The guide polynucleotide can be produced by any method known in the art, including chemically synthesizing guide polynucleotides (such as but not limiting to Hendel et al. 2015, Nature Biotechnology 33, 985-989), in vitro generated guide polynucleotides, and/or self-splicing guide RNAs (such as but not limiting to Xie et al. 2015, PNAS 112:3570-3575).

A method of expressing RNA components such as guide RNA in prokaryotic cells for performing Cas9-mediated DNA targeting have been described (WO2016/099887 published on Jun. 23, 2016 and WO2018/156705 published on Aug. 30, 2018)

In some aspects, a subject nucleic acid (e.g., a guide polynucleotide, a nucleic acid comprising a nucleotide sequence encoding a guide polynucleotide; a nucleic acid encoding Cas protein; a crRNA or a nucleotide encoding a crRNA, a tracrRNA or a nucleotide encoding a tracrRNA, a nucleotide encoding a VT domain, a nucleotide encoding a CPR domain, etc.) comprises a modification or sequence that provides for an additional desirable feature (e.g., modified or regulated stability; subcellular targeting; tracking, e.g., a fluorescent label; a binding site for a protein or protein complex; etc.). Nucleotide sequence modification of the guide polynucleotide, VT domain and/or CER domain can be selected from, but not limited to, the group consisting of a 5′ cap, a 3′ polyadenylated tail, a riboswitch sequence, a stability control sequence, a sequence that forms a dsRNA duplex, a modification or sequence that targets the guide poly nucleotide to a subcellular location, a modification or sequence that provides for tracking, a modification or sequence that provides a binding site for proteins, a Locked Nucleic Acid (LNA), a 5-methyl dC nucleotide, a 2,6-Diaminopurine nucleotide, a 2′-Fluoro A nucleotide, a 2′-Fluoro U nucleotide; a 2′-O-Methyl RNA nucleotide, a phosphorothioate bond, linkage to a cholesterol molecule, linkage to a polyethylene glycol molecule, linkage to a spacer 18 molecule, a 5′ to 3′ covalent linkage, or any combination thereof. These modifications can result in at least one additional beneficial feature, wherein the additional beneficial feature is selected from the group of a modified or regulated stability, a subcellular targeting, tracking, a fluorescent label, a binding site for a protein or protein complex, modified binding affinity to complementary target sequence, modified resistance to cellular degradation, and increased cellular permeability.

### Guided Cas Systems

The terms “guide RNA/Cas endonuclease complex”, “guide RNA/Cas endonuclease system”, “guide RNA/Cas complex”, “guide RNA/Cas system”, “gRNA/Cas complex”, “gRNA/Cas system”, “RNA-guided endonuclease”, “RGEN” are used interchangeably herein and refer to at least one RNA component and at least one Cas endonuclease, that are capable of forming a complex, wherein said guide RNA/Cas endonuclease complex can direct the Cas endonuclease to a DNA target site, enabling the Cas endonuclease to recognize, bind to, and optionally nick or cleave (introduce a single or double-strand break) the DNA target site

The present disclosure further provides expression constructs for expressing in a Bacillus sp. cell a guide RNA/Cas system that is capable of recognizing, binding to, and optionally nicking, unwinding, or cleaving all or part of a target sequence.

### Expression Cassettes and Recombinant DNA Constructs

Polynucleotides disclosed herein, such as a polynucleotide of interest a synthetic sequence of interest, a heterologous sequence of interest, a homologous sequence of interest, a gene of interest, can be provided in an expression cassette (also referred to as DNA construct) for expression in an organism of interest.

The term “expression”, as used herein, refers to the production of a functional end-product (e.g., a crRNA, a tracrRNA, a mRNA, a guide RNA, sRNA, siRNA, antisense RNA, or a polypeptide (protein) in either precursor or mature form. The term “expression” includes any step involved in the production of a polypeptide including, but not limited to, transcription, post-transcriptional modification, translation, post-translational modification, and secretion.

The expression cassette can include 5′ and 3′ regulatory sequences and or tags and synthetic sequences operably linked to a polynucleotide as disclosed herein.

The expression cassettes disclosed herein may include in the 5′-3′ direction of transcription, a transcriptional and translational initiation region (i.e., a promoter), a 5′ untranslated region, polynucleotides encoding various proteins tags and sequences, a polynucleotide of interest, and a transcriptional and translational termination region (i.e., termination region) functional in the Bacillus sp. (host) cell. Expression cassettes are also provided with a plurality of restriction sites and/or recombination sites for insertion of the polynucleotide to be under the transcriptional regulation of the regulatory regions described elsewhere herein. The regulatory regions (i.e., promoters, transcriptional regulatory regions, and translational termination regions) and/or the polynucleotide of interest may be native/analogous to the host cell or to each other. Other polynucleotide sequences encoding various protein sequences may be appended to either the 5′ or 3′ end of the polynucleotide of interest. Alternatively, the regulatory regions and/or the polynucleotide of interest may be heterologous to the host cell or to each other.

In certain embodiments the polynucleotides disclosed herein can be stacked with any combination of polynucleotide sequences of interest or expression cassettes as disclosed elsewhere herein or known in the art. The stacked polynucleotides may be operably linked to the same promoter as the initial polynucleotide, or may be operably linked to a separate promoter polynucleotide.

Expression cassettes may comprise a promoter operably linked to a polynucleotide of interest, along with a corresponding termination region. The termination region may be native to the transcriptional initiation region, may be native to the operably linked polynucleotide of interest or to the promoter sequences, may be native to the host organism, or may be derived from another source (i.e., foreign or heterologous). Convenient termination regions are available from phage sequences, eg. lambda phage t0 termination region or strong terminators from prokaryotic ribosomal RNA operons or genes involved in the secretion of extracellular proteins (eg. aprE from B. subtilis, aprL from B. licheniformis). Convenient termination regions are available from the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acids Res. 15:9627-9639.

Where appropriate, the polynucleotides of interest may be optimized for increased expression in the transformed or targeted organism. For example, the polynucleotides can be synthesized or altered to use organism-preferred codons for improved expression.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

The expression cassettes may additionally contain 5′ leader sequences. Such leader sequences can act to enhance translation or the level of RNA stability. 5′ leader sequences used interchangeably with 5′ untranslated regions could come from well-known and well characterized bacterial UTRs such as those from the Bacillus subtilis aprE gene or the Bacillus licheniformis amyL gene or any bacterial ribosomal protein gene. Translation leaders are known in the art and include: picornavirus leaders, for example, EMCV leader (Encephalomyocarditis 5′ noncoding region) (Elroy-Stein et al. (1989) Proc. Natl. Acad. Sci. USA 86:6126-6130); potyvirus leaders, for example, TEV leader (Tobacco Etch Virus) (Gallie et al. (1995) Gene 165(2):233-238), MDMV leader (Maize Dwarf Mosaic Virus) (Johnson et al. (1986) Virology 154:9-20), and human immunoglobulin heavy-chain binding protein (BiP) (Macejak et al. (1991) Nature 353:90-94); untranslated leader from the coat protein mRNA of alfalfa mosaic virus (AMV RNA 4) (Jobling et al. (1987) Nature 325:622-625); tobacco mosaic virus leader (TMV) (Gallie et al. (1989) in Molecular Biology of RNA, ed. Cech (Liss, New York), pp. 237-256); and maize chlorotic mottle virus leader (MCMV) (Lommel et al. (1991) Virology 81:382-385). See also, Della-Cioppa et al. (1987) Plant Physiol. 84:965-968. Other methods known to enhance translation can also be utilized, for example, introns, and the like.

In preparing the expression cassette, the various DNA fragments may be manipulated so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

In some embodiments, a nucleotide sequence encoding a guide RNA and/or a Cas protein is operably linked to a control element, e.g., a transcriptional control element, such as a promoter. The transcriptional control element may be functional in either a eukaryotic cell or a prokaryotic cell (e.g., bacterial or Bacillus sp. cell). Non-limiting examples of suitable prokaryotic promoters (promoters functional in a prokaryotic cell) and promoter sequence regions for use in the expression of genes, open reading frames (ORFs) thereof and/or variant sequences thereof in Bacillus sp. cells are generally known on one of skill in the art. Promoter sequences of the disclosure are generally chosen so that they are functional in the Bacillus sp. cells (e.g., B. licheniformis cells, B. subtilis cells and the like). Likewise, promoters useful for driving gene expression in Bacillus sp. cells include, but are not limited to, the promoters of the Bacillus licheniformis amylase gene (amyL), the promoters of the Bacillus stearothermophilus maltogenic amylase gene (amyM), the promoters of the Bacillus amyloliquefaciens amylase (amyQ), the promoters of the Bacillus subtilis xylA and xylB genes, the Bacillus subtilis alkaline protease (aprE) promoter (Stahl et al., 1984), the α-amylase promoter of Bacillus subtilis (Yang et al., 1983), the α-amylase promoter of Bacillus amyloliquefaciens (Tarkinen et al., 1983), the neutral protease (nprE) promoter from Bacillus subtilis (Yang et al., 1984), a mutant aprE promoter (PCT Publication No. WO2001/51643) or any other promoter from Bacillus licheniformis or other related Bacilli. In certain other embodiments, the promoter is a ribosomal protein promoter or a ribosomal RNA promoter (e.g., the rrnl promoter) disclosed in U.S. Patent Publication No. 2014/0329309. Synthetic promoters like spac can be both constitutive or inducible depending on other accessory factors. Phage promoters like n25, lambda pL or pR can be constitutive or inducible much in the same way. Methods for screening and creating promoter libraries with a range of activities (promoter strength) in Bacillus sp. cells is describe in PCT Publication No. WO2003/089604.

In some embodiments, a nucleotide sequence encoding a Cas9 endonuclease is operably linked to a constitutive promoter functional in a Bacillus sp. cell. Constitutive promoters functional in Bacillus sp. include, but are not limited to, the promoters of the Bacillus licheniformis amylase gene (amyL), the promoters of the Bacillus stearothermophilus maltogenic amylase gene (amyM), the promoters of the Bacillus amyloliquefaciens amylase (amyQ), the Bacillus subtilis alkaline protease (aprE) promoter, the α-amylase promoter of Bacillus subtilis (Yang et al., 1983), the α-amylase promoter of Bacillus amyloliquefaciens (Tarkinen et al., 1983), the neutral protease (nprE) promoter from Bacillus subtilis (Yang et al., 1984).

As used herein, “recombinant” refers to an artificial combination of two otherwise separated segments of sequence, e.g., by chemical synthesis or by the manipulation of isolated segments of nucleic acids by genetic engineering techniques. The term “recombinant,” when used in reference to a biological component or composition (e.g., a cell, nucleic acid, polypeptide/enzyme, vector, etc.) indicates that the biological component or composition is in a state that is not found in nature. In other words, the biological component or composition has been modified by human intervention from its natural state. For example, a recombinant cell encompasses a cell that expresses one or more genes that are not found in its native (i.e., non-recombinant) cell, a cell that expresses one or more native genes in an amount that is different than its native cell, and/or a cell that expresses one or more native genes under different conditions than its native cell. Recombinant nucleic acids may differ from a native sequence by one or more nucleotides, be operably linked to heterologous sequences (e.g., a heterologous promoter, a sequence encoding a non-native or variant signal sequence, etc.), be devoid of intronic sequences, and/or be in an isolated form. Recombinant polypeptides/enzymes may differ from a native sequence by one or more amino acids, may be fused with heterologous sequences, may be truncated or have internal deletions of amino acids, may be expressed in a manner not found in a native cell (e.g., from a recombinant cell that over-expresses the polypeptide due to the presence in the cell of an expression vector encoding the polypeptide), and/or be in an isolated form. It is emphasized that in some embodiments, a recombinant polynucleotide or polypeptide/enzyme has a sequence that is identical to its wild-type counterpart but is in a non-native form (e.g., in an isolated or enriched form).

As used herein, “recombinant DNA” or “recombinant DNA construct” refers to a DNA sequence comprising at least one expression cassette comprising an artificial combination of nucleic acid fragments. The recombinant DNA construct can include 5′ and 3′ regulatory sequences operably linked to a polynucleotide of interest as disclosed herein. For example, a recombinant DNA construct may comprise regulatory sequences and coding sequences that are derived from different sources. Such a recombinant DNA construct may be used by itself or it may be used in conjunction with a vector, which is referred to herein as a circular recombinant DNA construct. The choice of vector is dependent upon the method that will be used to introduce the vector into the host cells as is well known to those skilled in the art. For example, a plasmid vector can be used. The skilled artisan is well aware of the genetic elements that must be present on the vector in order to successfully transform, select and propagate host cells.

Standard recombinant DNA and molecular cloning techniques used herein are well known in the art and are described more fully in Sambrook et al., Molecular Cloning: A Laboratory Manual; Cold Spring Harbor Laboratory: Cold Spring Harbor, N.Y. (1989).

As used herein, “circular recombinant DNA construct” or “circular recombinant DNA” refers to a recombinant DNA construct that is circular. The term “circular recombinant DNA construct” includes a circular extra chromosomal element comprising autonomously replicating sequences, genome integrating sequences (such as but not limiting to single or multi-copy gene expression cassettes), phage, or nucleotide sequences, derived from any source, or synthetic (ie. not occurring in nature), in which a number of nucleotide sequences have been joined or recombined into a unique construction which is capable of introducing a polynucleotide of interest into a cell.

In one aspect the circular recombinant DNA construct comprises a vector backbone and a promoter sequences operably linked to a polynucleotide encoding a Cas endonuclease

In another aspect the circular recombinant DNA construct comprises a vector backbone and a first promoter operably linked to a polynucleotide of interest encoding a protein of interest and a second promoter operably linked to a polynucleotide encoding a guide RNA.

In some embodiments, the circular recombinant DNA construct comprises a vector backbone and a Cas9 endonuclease DNA encoding a Cas9 endonuclease operably linked to a constitutive promoter functional in a Bacillus sp. cell.

In one aspect, the circular recombinant DNA construct includes heterologous 5′ and 3′ regulatory sequences operably linked to a Cas9 endonuclease as disclosed herein. These regulatory sequences include but are not limited to a transcriptional and translational initiation region (i.e., a promoter), a nuclear localization signal, and a transcriptional and translational termination region (i.e., termination region) functional in a Bacillus sp. cell.

In one aspect, the recombinant DNA construct comprises a DNA encoding a Cas9 endonuclease described herein, wherein said Cas9 endonuclease is operably linked to or comprises a heterologous regulatory element such as a nuclear localization sequence (NLS).

In one aspect, the recombinant DNA construct comprises a DNA encoding Cas9 endonuclease described herein, wherein said Cas9 endonuclease is operably linked to or comprises a protein destabilization domain (eg. an intein or a deg tag).

In one aspect, the recombinant DNA construct comprises a DNA encoding Cas9 endonuclease described herein, wherein said Cas9 endonuclease is operably linked to or comprises a protein tag (eg. a poly histidine tag).

In one aspect, the recombinant DNA construct comprises a DNA encoding Cas9 endonuclease described herein, wherein said Cas9 endonuclease is operably linked to or comprises a fluorescent protein (eg. a GFP).

In one aspect, the recombinant DNA construct comprises a DNA encoding a Cas9 endonuclease described herein, wherein said Cas9 endonuclease is operably linked to or comprises a DNA binding domain (eg. mu gam, tetR).

### Target Sites

The terms “target site”, “target sequence”, “target site sequence, “target DNA”, “target locus”, “genomic target site”, “genomic target sequence”, “genomic target locus” and “protospacer”, are used interchangeably herein and refer to a polynucleotide sequence such as, but not limited to, a nucleotide sequence on a chromosome, episome, a transgenic locus, or any other DNA molecule in the genome (including chromosomal, plasmid DNA) of a cell, at which a guide polynucleotide/Cas endonuclease complex can recognize, bind to, and optionally nick or cleave.

The target site can be an endogenous site in the genome of a cell, or alternatively, the target site can be heterologous to the cell and thereby not be naturally occurring in the genome of the cell, or the target site can be found in a heterologous genomic location compared to where it occurs in nature. As used herein, terms “endogenous target sequence” and “native target sequence” are used interchangeable herein to refer to a target sequence that is endogenous or native to the genome of a cell and is at the endogenous or native position of that target sequence in the genome of the cell. An “artificial target site” or “artificial target sequence” are used interchangeably herein and refer to a target sequence that has been introduced into the genome of a cell. Such an artificial target sequence can be identical in sequence to an endogenous or native target sequence in the genome of a cell but be located in a different position (i.e., a non-endogenous or non-native position) in the genome of a cell.

An “altered target site”, “altered target sequence”, “modified target site”, “modified target sequence” are used interchangeably herein and refer to a target sequence as disclosed herein that comprises at least one alteration when compared to non-altered target sequence. Such “alterations” include, for example: (i) replacement of at least one nucleotide, (ii) a deletion of at least one nucleotide, (iii) an insertion of at least one nucleotide, or (iv) any combination of (i)-(iii).

The target site for a Cas endonuclease can be very specific and can often be defined to the exact nucleotide position, whereas in some cases the target site for a desired genome modification can be defined more broadly than merely the site at which DNA cleavage occurs, e.g., a genomic locus or region that is to be deleted from the genome. Thus, in certain cases, the genome modification that occurs via the activity of Cas/guide RNA DNA cleavage is described as occurring “at or near” the target site.

Methods for “modifying a target site” and “altering a target site” are used interchangeably herein and refer to methods for producing an altered target site.

A variety of methods are available to identify those cells having an altered genome at or near a target site without using a screenable marker phenotype. Such methods can be viewed as directly analyzing a target sequence to detect any change in the target sequence, including but not limited to PCR methods, sequencing methods, nuclease digestion, Southern blots, and any combination thereof.

The length of the target DNA sequence (target site) can vary, and includes, for example, target sites that are at least 12, 13, 14, 15, 16, 17, 18, 19, 20, 21, 22, 23, 24, 25, 26, 27, 28, 29, 30 or more nucleotides in length. It is further possible that the target site can be palindromic, that is, the sequence on one strand reads the same in the opposite direction on the complementary strand. The nick/cleavage site can be within the target sequence or the nick/cleavage site could be outside of the target sequence. In another variation, the cleavage could occur at nucleotide positions immediately opposite each other to produce a blunt end cut or, in other cases, the incisions could be staggered to produce single-stranded overhangs, also called “sticky ends”, which can be either 5′ overhangs, or 3′ overhangs. Active variants of genomic target sites can also be used. Such active variants can comprise at least 65%, 70%, 75%, 80%, 85%, 90%, 91%, 92%, 93%, 94%, 95%, 96%, 97%, 98%, 99% or more sequence identity to the given target site, wherein the active variants retain biological activity and hence are capable of being recognized and cleaved by a Cas endonuclease.

Assays to measure the single or double-strand break of a target site by an endonuclease are known in the art and generally measure the overall activity and specificity of the agent on DNA substrates containing recognition sites.

### Protospacer Adjacent Motif (PAM)

A “protospacer adjacent motif” (PAM) herein refers to a short nucleotide sequence adjacent to a target sequence (protospacer) that is recognized (targeted) by a guide polynucleotide/Cas endonuclease (PGEN) system. The Cas endonuclease may not successfully recognize a target DNA sequence if the target DNA sequence is not followed by a PAM sequence. The sequence and length of a PAM herein can differ depending on the Cas protein or Cas protein complex used. The PAM sequence can be of any length but is typically 1, 2, 3, 4, 5, 6, 7, 8, 9, 10, 11, 12, 13, 14, 15, 16, 17, 18, 19 or 20 nucleotides long.

A PAM herein is typically selected in view of the type of PGEN being employed. A PAM sequence herein may be one recognized by a PGEN comprising a Cas, such as the Cas9 variants described herein, derived from any of the species disclosed herein from which a Cas can be derived, for example. In certain embodiments, the PAM sequence may be one recognized by an RGEN comprising a Cas9 derived from S. pyogenes, S. thermophilus, S. agalactiae, N. meningitidis, T. denticola, or F. novicida. For example, a suitable Cas9 derived from S. pyogenes, Including the Cas9 Y155 variants described herein, could be used to target genomic sequences having a PAM sequence of NGG; N can be A, C, T, or G). As other examples, a suitable Cas9 could be derived from any of the following species when targeting DNA sequences having the following PAM sequences: S. thermophilus (NNAGAA), S. agalactiae (NGG), NNAGAAW [W is A or T], NGGNG), N. meningitidis (NNNNGATT), T. denticola (NAAAAC), or F. novicida (NG) (where N's in all these particular PAM sequences are A, C, T, or G). Other examples of Cas9/PAMs useful herein include those disclosed in Shah et al. (RNA Biology 10:891-899) and Esvelt et al. (Nature Methods 10:1116-1121), which are incorporated herein by reference.

### Dual Circular Recombinant DNA Systems for Efficient Polynucleotide Integration in Bacillus sp.

The presently disclosed circular recombinant DNA constructs can be introduced into a Bacillus sp. cell.

The methods described herein employ a dual circular recombinant DNA system for introduction of a guide RNA/Cas endonuclease system (RGEN) as well as a donor DNA (comprising the polynucleotide of interest) into a Bacillus sp. cell, and providing a highly effective system for integrating polynucleotides of interest into a target site on the genome of said Bacillus sp. cell, without the need to integrate a selectable marker in the genome of said Bacillus sp. cell.

Applicants have surprisingly and unexpectedly found that when two circular recombinant DNA constructs, having a first circular recombinant DNA comprising a donor DNA sequence flanked by homology arms of 600 bps, and a second circular recombinant DNA having a Cas9 endonuclease expression cassette, are simultaneously introduced into a Bacillus sp. cell without the introduction of a selectable marker into said genome (herein referred to as a dual circular recombinant DNA system), an increased efficiency in gene integration is observed, when compared to a control system having a (first) linear donor DNA flanked by two homology arms of 1000 bps, and having a (second) circular recombinant DNA construct comprising said DNA sequence encoding said guide RNA and comprising said Cas9 endonuclease DNA sequence operably linked to a constitutive promoter.

In one aspect the dual circular recombinant DNA systems comprises the simultaneous introduction of a first circular recombinant DNA construct and a second circular recombinant DNA construct into a Bacillus sp. cell, wherein said first circular recombinant DNA construct comprises a DNA sequence encoding a guide RNA and a donor DNA sequence comprising a gene of interest encoding a protein of interest, wherein said second circular recombinant DNA construct comprises a Cas9 endonuclease DNA sequence operably linked to a constitutive promoter, wherein said Cas9 endonuclease DNA sequence encodes a Cas9 endonuclease introduces a double-strand break at or near a target site in the genome of said Bacillus sp. cell, wherein no selectable marker is integrated into the genome of said Bacillus sp. cell. The donor DNA sequence can be flanked by two homology arms, one upstream arm (5′ HR1) and one downstream arm (3′ HR2) wherein each homology arm is between 70 nucleotides and 600 nucleotides, between 100 and 600 nucleotides, between 200 and 600 nucleotides, between 300 and 600 nucleotides, between 400 and 600 nucleotides, between 500 and 600 nucleotides, up to 600 nucleotides in length and comprises sequence homology to a targeted genomic locus of said Bacillus sp. cell.

In one aspect, the method described herein comprises a method for integrating a gene of interest into a target site on the genome of a Bacillus sp. cell without the integration of a selectable marker into said genome, the method comprising simultaneously introducing at least a first circular recombinant DNA construct and a second circular recombinant DNA construct into a Bacillus sp. cell, wherein said first circular recombinant DNA construct comprises a donor DNA sequence comprising a gene of interest and a DNA sequence encoding a guide RNA, wherein said second circular recombinant DNA construct comprises a Cas9 endonuclease DNA sequence operably linked to a constitutive promoter, wherein said Cas9 endonuclease DNA sequence encodes a Cas9 that introduces a double-strand break at or near a target site in the genome of said Bacillus sp. cell., wherein the donor DNA sequence is flanked by two homology arms, one upstream homology arm (5′ HR1) and one downstream homology arm (3′ HR2) wherein each homology arm is equal to 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, or up to 600 nucleotides in length, and comprises sequence homology to said target site on the genome of the Bacillus sp. cell.

Previous methods for gene integration into the genome of Bacillus sp. cells relied on spontaneous double strand break occurrence and use of selectable markers co-located on linear DNA fragments with short homology arms (comprising both the gene of interest (GOI) to be inserted into the genome as well as a selectable marker that was also inserted into the genome to enable identification of Bacillus sp. cells that had the gene of interest integrated into its genome (WO02/14490, published on Feb. 21, 2002). The selectable marker and GOI were typically flanked by two short homology arms such that upon recombination with the DNA within the cell both the GOI and the selectable marker would be integrated in the DNA of the cell. The use of selectable markers during transformation of such linear fragments with short homology arms for genome integration into Bacillus cells is required to select for efficient modification of a specific locus of the genome. The marker must integrate into the correct locus for expression and this integration relies on rare, spontaneous DNA damage that occurs in a stoichastic manner within the population and within the genome. This rare event can only be selected for by combining the use of a marker and chromosomal integration. (WO02/14490, published on Feb. 21, 2002).

In contrast, the present disclosure describes a method for generating site specific DNA double strand breaks (DNA damage) that essentially converts a majority of the population to cells which containing said DNA damage at the desired locus and as such does not rely on a rare spontaneous DNA damage. Hence, generating DNA double strand breaks is no longer the limiting step for modifying a chromosomal locus (as is the case in WO02/14490, published on February 21, 200), instead the present disclose only optionally uses selectable markers (located on the recombinant DNA constructs) to differentiate transformed from non-transformed cells solely to enable increased transformation efficiency. In case any one of the recombinant DNA constructs of the disclosed dual circular recombinant DNA system comprise a selectable marker, the recombinant DNA construct (ans as such the selectable marker) do not integrate into the genome of the Bacillus sp. cells and progeny Bacillus sp. cells can be selected that do not contain said selectable marker integrate into their genome.

The dual circular recombinant DNA system described herein has a further advantage in that is discloses the simultaneous introduction of a pCas9 plasmid and a plasmid comprising the donor DNA (polynucleotide of interest) without the need for expression of the RED recombination system, rather than sequential introduction of plasmids described in E. coli systems using a method relying on the sequential introduction of a pCas9 plasmid followed by a pTARGET plasmid and the need to express the RED recombination system (described in Jiang et al. 2015, Applied and Environmental Microbiology, volume 81, nr. 7, pg 2506-2514). In addition to the sequential introduction of the pCas9 and pTARGET plasmid, Jiang et al. 2015 disclose the need to express the RED recombination system of bacteriophage A for any repair from editing templates to occur within their system. Therefore, as described herein, it is surprising that in Bacillus sp. that efficient repair is seen with no need for expression of the RED recombination system.

One of the bottlenecks in development of Bacillus sp. hosts for enzyme production is an antibiotic resistant marker (ARM)-free integration of multi-copy enzyme expression cassettes in the chromosome. Existing approaches such as using an integration vector, Cre/loxP system, and auxotrophic marker are time consuming, and the editing efficiencies are relatively low.

In one aspect, the method described herein comprises a method for In one aspect, the method described herein comprises a method for integrating multiple copies of a gene expression cassette into the genome of a Bacillus sp. cell without the integration of a selectable marker into said genome, the method comprising simultaneously introducing at least a first circular recombinant DNA construct and a second circular recombinant DNA construct into a Bacillus sp. cell, wherein said first circular recombinant DNA construct comprises a donor DNA sequence comprising multiple copies of a gene expression cassette, wherein each gene expression cassette comprising the same gene of interest and a DNA sequence encoding a guide RNA, wherein said second circular recombinant DNA construct comprises a Cas9 endonuclease DNA sequence operably linked to a constitutive promoter, wherein said Cas9 endonuclease DNA sequence encodes a Cas9 that introduces a double-strand break at or near a target site in the genome of said Bacillus sp. cell. The donor DNA sequence can be flanked by two homology arms, one upstream arm (5′ HR1) and one downstream arm (3′ HR2) wherein each homology arm is equal to about 70, 100, 200, 300, 400, 500, or up to 600 nucleotides in length and comprises sequence homology to a targeted genomic locus of said Bacillus sp. cell. In one aspect, the donor DNA sequence is flanked by an upstream (HR1) and downstream (HR2) homology arm of 600 bps or less. In one aspect, the multiple copies of said gene expression cassette are selected from the group consisting of 2 copies, 3 copies, 4 copies, 5 copies, 6 copies, 7 copies, 8 copies, 9 copies and up to 10 copies.

In one embodiment, the disclosure comprises a method for integrating a gene of interest into a target site on the genome of a Bacillus sp. cell without the integration of a selectable marker into said genome, the method comprising simultaneously introducing at least a first circular recombinant DNA construct and a second circular recombinant DNA construct into a Bacillus sp. cell, wherein said first circular recombinant DNA construct comprises a donor DNA sequence comprising a gene of interest and a DNA sequence encoding a guide RNA, wherein said second circular recombinant DNA construct comprises a Cas9 endonuclease DNA sequence operably linked to a constitutive promoter, wherein said Cas9 endonuclease DNA sequence encodes a Cas9 that introduces a double-strand break at or near a target site in the genome of said Bacillus sp. cell. The donor DNA sequence can be flanked by two homology arms, one upstream arm (5′ HR1) and one downstream arm (3′ HR2) wherein each homology arm is between 70 nucleotides and 600 nucleotides, between 100 and 600 nucleotides, between 200 and 600 nucleotides, between 300 and 600 nucleotides, between 400 and 600 nucleotides, between 500 and 600 nucleotides, and up to 600 nucleotides in length, and comprises sequence homology to a targeted genomic locus of said Bacillus sp. cell.

In one aspect, the first and/or second circular recombinant DNA construct comprise a selectable marker that is used to facilitate selection of transformed Bacillus sp. cells, but is not necessary for selection of (daughter) Bacillus sp. cells that have the gene of interest integrated into its genome. These daughter Bacillus sp. cells have lost the first and second circular recombinant DNA construct comprising the selectable maker, and as such have no selectable marker integrated into their genome. As such, the method can further comprise growing progeny cells from said Bacillus sp. cell and selecting a Bacillus sp. progeny cell that does not contain the first and/or second circular recombinant DNA construct (and does not contain the selectable marker comprised on these circular recombinant DNAs) but has the gene of interest stably integrated in its genome.

In some embodiments, the method described above results in a frequency of integration of the gene of interest gene (into the Bacillus sp. genome) that is at least about 2, 3, 4, 5, 6, 7, 8, 9, 10 up to 11 fold higher when compared to the frequency of integration of a control method comprising introducing into a Bacillus sp. cell a linear recombinant DNA construct comprising said donor DNA sequence flanked by an upstream (5′ HR1) and downstream homology arm (3′ HR2) of 1000 bps, and a circular recombinant DNA construct comprising said DNA sequence encoding said guide RNA and said Cas9 endonuclease DNA sequence operably linked to a constitutive promoter.

The terms “knock-in”, “gene knock-in, “gene insertion” and “genetic knock-in” are used interchangeably herein. A knock-in represents the replacement or insertion of a DNA sequence at a specific DNA sequence in cell by targeting with a Cas protein (for example by homologous recombination (HR), wherein a suitable donor DNA polynucleotide is also used). Examples of knock-ins are a specific insertion of a heterologous amino acid coding sequence in a coding region of a gene, or a specific insertion of a transcriptional regulatory element in a genetic locus.

The dual circular recombinant DNA system described herein can be used as a method for integrating a polynucleotide or gene of interest into the genome of a Bacillus sp. cell.

In one aspect, this method employs homologous recombination (HR) to provide integration of the polynucleotide or gene of interest at the target site.

As used herein, “donor DNA” and “donor DNA sequence” refers to a DNA sequence that comprises a polynucleotide of interest to be inserted into the target site of a Cas endonuclease. The donor DNA sequence (such as but not limiting to a gene of interest) can be flanked by a first (HR1) and a second (HR2) region of homology (also referred to as homology arm). The first and second regions of homology of the donor DNA share homology to a first and a second genomic region, respectively, present in or flanking the target site of the cell or organism genome.

As used herein, “homology arm” refers to a nucleic acid sequence, which is homologous to a sequence in the Bacillus genome. More specifically, a homology arm is an upstream or downstream region having between about 80 and 100% sequence identity, between about 90 and 100% sequence identity, or between about 95 and 100% sequence identity with the immediate flanking region of a target sequence.

The homology arms of the present disclosure, flanking a donor DNA sequence, located in a circular recombinant DNA described herein, include about is between 70 nucleotides and 600 nucleotides, between 100 and 600 nucleotides, between 200 and 600 nucleotides, between 300 and 600 nucleotides, between 400 and 600 nucleotides, between 500 and 600 nucleotides, and up to 600 nucleotides in length.

In some embodiments, the 5′ and 3′ ends of a gene of interest are flanked by a homology arm wherein the homology arm comprises nucleic acid sequences immediately flanking the targeted genomic locus of the Bacillus sp. cell.

In some embodiments, the donor DNA sequence is flanked by two homology arms, one located at its 5′ end (e.g., up-stream homology arm) and one at its 3′ end (e.g., down-stream homology arm).

In some embodiments, the donor DNA sequence located in a circular recombinant DNA of the disclosure is flanked by two homology arms, one upstream arm (5′ HR1) and one downstream arm (3′ HR2) wherein each homology arm is between 70 nucleotides and 600 nucleotides, between 100 and 600 nucleotides, between 200 and 600 nucleotides, between 300 and 600 nucleotides, between 400 and 600 nucleotides, between 500 and 600 nucleotides, and up to 600 nucleotides in length and comprises sequence homology to a targeted genomic locus of said Bacillus sp. cell.

In one embodiment, the disclosure comprises a method for integrating a gene of interest into a target site on the genome of a Bacillus sp. cell without the introduction of a selectable marker into said genome, the method comprising simultaneously introducing at least a first circular recombinant DNA construct and a second circular recombinant DNA construct into a Bacillus sp. cell, wherein said first circular recombinant DNA construct comprises a donor DNA sequence comprising a gene of interest and a DNA sequence encoding a guide RNA, wherein said second circular recombinant DNA construct comprises a Cas9 endonuclease DNA sequence operably linked to a constitutive promoter, wherein said Cas9 endonuclease DNA sequence encodes a Cas9 that introduces a double-strand break at or near a target site in the genome of said Bacillus sp. cell.

The donor DNA sequence can be flanked by two homology arms, one upstream arm (5′ HR1) and one downstream arm (3′ HR2) wherein each homology arm is between 70 nucleotides and 600 nucleotides, between 100 and 600 nucleotides, between 200 and 600 nucleotides, between 300 and 600 nucleotides, between 400 and 600 nucleotides, between 500 and 600 nucleotides, or up to 600 nucleotides in length and comprises sequence homology to a targeted genomic locus of said Bacillus sp. cell.

The method can further comprise growing progeny cells from said Bacillus sp. cell and selecting a Bacillus sp. progeny cell that does not contain the first and/or second circular recombinant DNA construct (and does not contain the selectable marker comprised on these circular recombinant DNAs) but has the gene of interest stably integrated in its genome.

As described herein, such a method can result in a frequency of integration of the gene of interest gene that is at least about 2, 3, 4, 5, 6, 7, 8, 9, 10 up to 11 fold higher when compared to the frequency of integration a control method comprising introducing into a Bacillus sp. cell a linear recombinant DNA construct comprising said donor DNA sequence flanked by an upstream (5′ HR1) and downstream homology arm (3′ HR2) of 1000 bps, and a circular recombinant DNA construct comprising said DNA sequence encoding said guide RNA and said Cas9 endonuclease DNA sequence operably linked to a constitutive promoter.

In some embodiments, the first circular recombinant DNA construct and/or the second circular recombinant DNA construct comprise an autonomous replicating sequence.

In some embodiments, the method is a method for integrating a gene of interest into a target site on the genome of a Bacillus sp. cell without the integration of a selectable marker into said genome, the method comprising simultaneously introducing at least a first circular recombinant DNA construct and a second circular recombinant DNA construct into a Bacillus sp. cell, wherein said first circular recombinant DNA construct comprises a donor DNA sequence comprising a gene of interest and a DNA sequence encoding a guide RNA, wherein said second circular recombinant DNA construct comprises a Cas9 endonuclease DNA sequence operably linked to a constitutive promoter, wherein said Cas9 endonuclease DNA sequence encodes a Cas9 that introduces a double-strand break at or near a target site in the genome of said Bacillus sp. cell, wherein the first and/or second circular recombinant DNA construct comprise an autonomous replicating sequence and a selectable marker.

In some embodiments, the method is a method for integrating a gene of interest into a target site on the genome of a Bacillus sp. cell without the integration of a selectable marker into said genome, the method comprising simultaneously introducing at least a first circular recombinant DNA construct and a second circular recombinant DNA construct into a Bacillus sp. cell, wherein said first circular recombinant DNA construct comprises a donor DNA sequence comprising a gene of interest and a DNA sequence encoding a guide RNA, wherein said second circular recombinant DNA construct comprises a Cas9 endonuclease DNA sequence operably linked to a constitutive promoter, wherein said Cas9 endonuclease DNA sequence encodes a Cas9 that introduces a double-strand break at or near a target site in the genome of said Bacillus sp. cell, wherein the first circular recombinant DNA construct is a low copy plasmid.

In some embodiments, the first circular recombinant DNA construct comprising a donor DNA sequence comprising a gene of interest and a DNA sequence encoding a guide RNA is a low copy plasmid.

Episomal DNA molecules can also be ligated into the double-strand break, for example, integration of T-DNAs into chromosomal double-strand breaks (Chilton and Que, (2003) Plant Physiol 133:956-65; Salomon and Puchta, (1998) EMBO J 17:6086-95). Once the sequence around the double-strand breaks is altered, for example, by exonuclease activities involved in the maturation of double-strand breaks, gene conversion pathways can restore the original structure if a homologous sequence is available, such as a homologous chromosome in non-dividing somatic cells, or a sister chromatid after DNA replication (Molinier et al., 2004, Plant Cell 16:342-52). Ectopic and/or epigenic DNA sequences may also serve as a DNA repair template for homologous recombination (Puchta, (1999) Genetics 152:1173-81).

Homology-directed repair (HDR) is a mechanism in cells to repair double-stranded and single stranded DNA breaks. Homology-directed repair includes homologous recombination (HR) and single-strand annealing (SSA) (Lieber. 2010 Annu. Rev. Biochem. 79:181-211). The most common form of HDR is called homologous recombination (HR), which has the longest sequence homology requirements between the donor and acceptor DNA. Other forms of HDR include single-stranded annealing (SSA) and breakage-induced replication, and these require shorter sequence homology relative to HR. Homology-directed repair at nicks (single-stranded breaks) can occur via a mechanism distinct from HDR at double-strand breaks (Davis and Maizels. PNAS (0027-8424), 111 (10), p. E924-E932).

By “homology” is meant DNA sequences that are similar. For example, a “region of homology to a genomic region” that is found on the donor DNA is a region of DNA that has a similar sequence to a given “genomic region” in the cell or organism genome. A region of homology can be of any length that is sufficient to promote homologous recombination at the cleaved target site. For example, the region of homology can comprise at least 5-10, 5-15, 5-20, 5-25, 5-30, 5-35, 5-40, 5-45, 5-50, 5-55, 5-60, 5-65, 5-70, 5-75, 5-80, 5-85, 5-90, 5-95, 5-100, 5-200, 5-300, 5-400, 5-500, 5-600, 5-700, 5-800, 5-900, 5-1000, 5-1100, 5-1200, 5-1300, 5-1400, 5-1500, 5-1600, 5-1700, 5-1800, 5-1900, 5-2000, 5-2100, 5-2200, 5-2300, 5-2400, 5-2500, 5-2600, 5-2700, 5-2800, 5-2900, 5-3000, 5-3100 or more bases in length such that the region of homology has sufficient homology to undergo homologous recombination with the corresponding genomic region. “Sufficient homology” indicates that two polynucleotide sequences have sufficient structural similarity to act as substrates for a homologous recombination reaction. The structural similarity includes overall length of each polynucleotide fragment, as well as the sequence similarity of the polynucleotides. Sequence similarity can be described by the percent sequence identity over the whole length of the sequences, and/or by conserved regions comprising localized similarities such as contiguous nucleotides having 100% sequence identity, and percent sequence identity over a portion of the length of the sequences.

The amount of homology or sequence identity shared by a target and a donor polynucleotide can vary and includes total lengths and/or regions having unit integral values in the ranges of about 1-20 bp, 20-50 bp, 50-100 bp, 75-150 bp, 100-250 bp, 150-300 bp, 200-400 bp, 250-500 bp, 300-600 bp, 350-750 bp, 400-800 bp, 450-900 bp, 500-1000 bp, 600-1250 bp, 700-1500 bp, 800-1750 bp, 900-2000 bp, 1-2.5 kb, 1.5-3 kb, 2-4 kb, 2.5-5 kb, 3-6 kb, 3.5-7 kb, 4-8 kb, 5-10 kb, or up to and including the total length of the target site. These ranges include every integer within the range, for example, the range of 1-20 bp includes 1, 2, 3, 4, 5, 6, 7, 8, 9, 10, 11, 12, 13, 14, 15, 16, 17, 18, 19 and 20 bps. The amount of homology can also be described by percent sequence identity over the full aligned length of the two polynucleotides which includes percent sequence identity of about at least 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. Sufficient homology includes any combination of polynucleotide length, global percent sequence identity, and optionally conserved regions of contiguous nucleotides or local percent sequence identity, for example sufficient homology can be described as a region of 75-150 bp having at least 80% sequence identity to a region of the target locus. Sufficient homology can also be described by the predicted ability of two polynucleotides to specifically hybridize under high stringency conditions, see, for example, Sambrook et al., (1989) Molecular Cloning: A Laboratory Manual, (Cold Spring Harbor Laboratory Press, NY); Current Protocols in Molecular Biology, Ausubel et al., Eds (1994) Current Protocols, (Greene Publishing Associates, Inc. and John Wiley & Sons, Inc.); and, Tijssen (1993) Laboratory Techniques in Biochemistry and Molecular Biology—Hybridization with Nucleic Acid Probes, (Elsevier, New York).

As used herein, a “genomic region” is a segment of a chromosome in the genome of a cell that is present on either side of the target site or, alternatively, also comprises a portion of the target site. The genomic region can comprise at least 5-10, 5-15, 5-20, 5-25, 5-30, 5-35, 5-40, 5-45, 5-50, 5-55, 5-60, 5-65, 5-70, 5-75, 5-80, 5-85, 5-90, 5-95, 5-100, 5-200, 5-300, 5-400, 5-500, 5-600, 5-700, 5-800, 5-900, 5-1000, 5-1100, 5-1200, 5-1300, 5-1400, 5-1500, 5-1600, 5-1700, 5-1800, 5-1900, 5-2000, 5-2100, 5-2200, 5-2300, 5-2400, 5-2500, 5-2600, 5-2700, 5-2800. 5-2900, 5-3000, 5-3100 or more bases such that the genomic region has sufficient homology to undergo homologous recombination with the corresponding region of homology.

The structural similarity between a given genomic region and the corresponding region of homology found on the donor DNA can be any degree of sequence identity that allows for homologous recombination to occur. For example, the amount of homology or sequence identity shared by the “region of homology” of the donor DNA and the “genomic region” of the organism genome can be at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity, such that the sequences undergo homologous recombination

The region of homology on the donor DNA can have homology to any sequence flanking the target site. While in some instances the regions of homology share significant sequence homology to the genomic sequence immediately flanking the target site, it is recognized that the regions of homology can be designed to have sufficient homology to regions that may be further 5′ or 3′ to the target site. The regions of homology can also have homology with a fragment of the target site along with downstream genomic regions

In one embodiment, the first region of homology further comprises a first fragment of the target site and the second region of homology comprises a second fragment of the target site, wherein the first and second fragments are dissimilar.

As used herein, “homologous recombination” includes the exchange of DNA fragments between two DNA molecules at the sites of homology. The frequency of homologous recombination is influenced by a number of factors. Different organisms vary with respect to the amount of homologous recombination and the relative proportion of homologous to non-homologous recombination. The length of the homology region (homology arm) needed to observe homologous recombination varies among organisms.

As described herein, Applicants have surprisingly and unexpectedly identified that when two circular recombinant DNA constructs (with one circular recombinant DNA construct comprising a donor DNA sequence comprising a gene of interest (wherein said donor DNA is flanked by two homology arms, one upstream arm (5′ HR1) and one downstream arm (3′ HR2) wherein each homology arm is between 70 nucleotides and 600 nucleotides, between 100 and 600 nucleotides, between 200 and 600 nucleotides, between 300 and 600 nucleotides, between 400 and 600 nucleotides, between 500 and 600 nucleotides, or up to 600 nucleotides in length, and comprises sequence homology to a targeted genomic locus of said Bacillus sp. cell.) are simultaneously introduced into a Bacillus sp. cell, an increased efficiency in gene integration is observed, when compared to a control system.

The homology arms of the present disclosure, flanking a donor DNA sequence, located in a circular recombinant DNA described herein, include about between 1 base pair (bp) and 70, between, 1 base pair (bp) and 100 bp; between 1 bp and 200 bp; between 1 bp and 300 bp; between 1 bp and 400 bp; between 1 bp and 500 bp and between 1 bp and 600 bp.

Alteration of the genome of a prokaryotic or organism cell, for example, through homologous recombination (HR), is a powerful tool for genetic engineering. Homologous recombination has also been accomplished in other organisms. For example, at least 150-200 bp of homology was required for homologous recombination in the parasitic protozoan Leishmania (Papadopoulou and Dumas, (1997) Nucleic Acids Res 25:4278-86) and 150-200 bp of homology is required for efficient recombination in the protobacterium E coli (Lovett et al (2002) Genetics 160:851-859). In Bacillus cells homology lengths of as little as 70 bp can be involved in homologous recombination but homology arm lengths of 25 bp cannot (Kahsanov F K et al Mol Gen Genetics (1992) 234:494-497).

### Introducing Multiple Copies of a Gene Expression Cassette

A multi-copy gene expression cassette or multi-copy expression cassette are used interchangeably herein and refer to multiple copies of the same expression cassette comprising at least one gene of interest. In one aspect, the multiple copies of said gene expression cassette are selected from the group consisting of 2 copies, 3 copies, 4 copies, 5 copies, 6 copies, 7 copies, 8 copies, 9 copies and up to 10 copies.

The single copy and/or multi-copy polynucleotide expression cassettes can be antibiotic resistant marker free (ARM-free) expression cassettes that are integrated into a plasmid such as a low-copy number plasmid.

In one aspect, the method described herein comprises a method for integrating multiple copies of a gene expression cassette into a target site on the genome of a Bacillus sp. cell without the integration of a selectable marker into said genome, the method comprising simultaneously introducing at least a first circular recombinant DNA construct and a second circular recombinant DNA construct into a Bacillus sp. cell, wherein said first circular recombinant DNA construct comprises a donor DNA sequence comprising multiple copies of a gene expression cassette, wherein each gene expression cassette comprising the same gene of interest and a DNA sequence encoding a guide RNA, wherein said second circular recombinant DNA construct comprises a Cas9 endonuclease DNA sequence operably linked to a constitutive promoter, wherein said Cas9 endonuclease DNA sequence encodes a Cas9 that introduces a double-strand break at or near a target site in the genome of said Bacillus sp. cell. The donor DNA sequence can be flanked by two homology arms, one upstream arm (5′ HR1) and one downstream arm (3′ HR2) wherein each homology arm is between 70 nucleotides and 600 nucleotides, between 100 and 600 nucleotides, between 200 and 600 nucleotides, between 300 and 600 nucleotides, between 400 and 600 nucleotides, between 500 and 600 nucleotides, up to 600 nucleotides in length, and comprises sequence homology to a targeted genomic locus of said Bacillus sp. cell. In one aspect, the multiple copies of said gene expression cassette are selected from the group consisting of 2 copies, 3 copies, 4 copies, 5 copies, 6 copies, 7 copies, 8 copies, 9 copies and up to 10 copies.

### Multiplexing

A targeting method herein can be performed in such a way that two or more DNA target sites are targeted in the method, for example. Such a method can optionally be characterized as a multiplex method. Two, three, four, five, six, seven, eight, nine, ten, or more target sites can be targeted at the same time in certain embodiments. A multiplex method is typically performed by a targeting method herein in which multiple different RNA components are provided, each designed to guide a guide polynucleotide/Cas endonuclease complex to a unique DNA target site.

### Definitions

Unless defined otherwise, all technical and scientific terms used herein have the same meaning as commonly understood by one of ordinary skill in the art to which the present compositions and methods apply.

An “allele” or “allelic variant” is one of several alternative forms of a gene occupying a given locus on a chromosome. When all the alleles present at a given locus on a chromosome are the same, that organism is homozygous at that locus. If the alleles present at a given locus on a chromosome differ, that organism is heterozygous at that locus. An allelic variant of a polypeptide is a polypeptide encoded by an allelic variant of a gene.

As used herein, “host cell” refers to a cell that has the capacity to act as a host or expression vehicle for a newly introduced DNA sequence. Thus, in certain embodiments of the disclosure, the host cells are Bacillus sp. cells.

A “recombinant host cell” (also referred to as a “genetically modified host cell”) is a host cell into which has been introduced a heterologous nucleic acid, e.g., a recombinant DNA construct, or which has been introduced and comprises a genome modification system such as the guide RNA/Cas endonuclease system described herein. For example, a subject bacterial host cell includes a genetically modified Bacillus sp. cell by virtue of introduction into a suitable Bacillus sp. cell of an exogenous nucleic acid (e.g., a plasmid or circular recombinant DNA construct).

As defined herein, a “parental cell” or a “parental (host) cell” may be used interchangeably and refer to “unmodified” parental cells. For example, a “parental” cell refers to any cell or strain of microorganism in which the genome of the “parental” cell is altered (e.g., via one or more mutations/modifications introduced into the parental cell) to generate a modified “daughter” cell thereof.

As used herein, a “modified cell” or a “modified (host) cell” may be used interchangeably and refer to recombinant (host) cells that comprise at least one genetic modification which is not present in the “parental” host cell from which the modified cells are derived.

As used herein, “the genus Bacillus” or “Bacillus sp.” cells include all species within the genus “Bacillus”′ as known to those of skill in the art, including but not limited to Bacillus subtilis, Bacillus licheniformis, Bacillus lentus, Bacillus brevis, Bacillus stearothermophilus, Bacillus alkalophilus, Bacillus amyloliquefaciens, Bacillus clausii, Bacillus. halodurans, Bacillus. megaterium, Bacillus coagulans, Bacillus circulans, Bacillus lautus, and Bacillus thuringiensis. It is recognized that the genus Bacillus continues to undergo taxonomical reorganization. Thus, it is intended that the genus include species that have been reclassified, including but not limited to such organisms as B. stearothermophilus, which is now named “Geobacillus stearothermophilus”.

The term “increased” as used herein may refer to a quantity or activity that is at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 100%, or at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 440, 450, 460, 470, 480, 490, or 500 fold more than the quantity or activity for which the increased quantity or activity is being compared. The terms “increased”, “greater than”, and “improved” are used interchangeably herein. The term “increased” can be used to characterize the transformation or gene editing efficiency obtained by a multicomponent method described herein when compared to a control method described herein,

In one aspect the increase is an increase in integration efficiency of a gene of interest into a Bacillus sp. cell obtained by the dual circular recombinant DNA system, described herein compared to the integration efficiency of a gene of interest into a Bacillus sp. cell obtained by the control recombinant DNA system described herein. In one aspect the increase is an increase in integration frequency of at least about 2, 3, 4, 5, 6, 7, 8, 9, 10, and up to or greater than 11 fold.

As used herein, the term “integration efficiency” is defined by diving the number of transformed cells having the desired donor DNA (gene of interest) integrated into its genome by the total number of transformed cells. This number can be multiplied by 100 to express it as a %.

Integration efficiency (%)=(number of transformed cells having donor DNA (gene of interest) integrated in its genome/number of total transformed cells)*100

The term “conserved domain” or “motif” means a set of amino acids conserved at specific positions along an aligned sequence of evolutionarily related proteins. While amino acids at other positions can vary between homologous proteins, amino acids that are highly conserved at specific positions indicate amino acids that are essential to the structure, the stability, or the activity of a protein. Because they are identified by their high degree of conservation in aligned sequences of a family of protein homologues, they can be used as identifiers, or “signatures”, to determine if a protein with a newly determined sequence belongs to a previously identified protein family.

As used herein, “nucleic acid” means a polynucleotide and includes a single or a double-stranded polymer of deoxyribonucleotide or ribonucleotide bases. Nucleic acids may also include fragments and modified nucleotides. Thus, the terms “polynucleotide”, “nucleic acid sequence”, “nucleotide sequence” and “nucleic acid fragment” are used interchangeably to denote a polymer of RNA and/or DNA and/or RNA-DNA that is single- or double-stranded, optionally containing synthetic, non-natural, or altered nucleotide bases. Nucleotides (usually found in their 5′-monophosphate form) are referred to by their single letter designation as follows: “A” for adenosine or deoxyadenosine (for RNA or DNA, respectively), “C” for cytosine or deoxycytosine, “G” for guanosine or deoxyguanosine, “U” for uridine, “T” for deoxythymidine, “R” for purines (A or G), “Y” for pyrimidines (C or T), “K” for G or T, “H” for A or C or T, “I” for inosine, and “N” for any nucleotide (nucleotide (e.g., N can be A, C, T, or G, if referring to a DNA sequence; N can be A, C, U, or G, if referring to an RNA sequence).

It is understood that the polynucleotides (or nucleic acid molecules) described herein include “genes”, “vectors” and “plasmids”.

The term “gene”, refers to a polynucleotide that codes for a functional molecule such as, but not limited to, a particular sequence of amino acids, which comprise all, or part of a protein coding sequence, and may include regulatory (non-transcribed) sequences, such as promoter sequences, which determine for example the conditions under which the gene is expressed. The transcribed region of the gene may include untranslated regions (UTRs), including introns, 5′-untranslated regions (UTRs), and 3′-UTRs, as well as the coding sequence. “Native gene” refers to a gene as found in nature with its own regulatory sequences.

A “codon-modified gene” or “codon-preferred gene” or “codon-optimized gene” is a gene having its frequency of codon usage designed to mimic the frequency of preferred codon usage of the host cell. The nucleic acid changes made to codon-optimize a gene are “synonymous”, meaning that they do not alter the amino acid sequence of the encoded polypeptide of the parent gene. However, both native and variant genes can be codon-optimized for a particular host cell, and as such no limitation in this regard is intended. Methods are available in the art for synthesizing codon-preferred genes. See, for example, U.S. Pat. Nos. 5,380,831, and 5,436,391, and Murray et al. (1989) Nucleic Acids Res. 17:477-498, herein incorporated by reference.

Additional sequence modifications are known to enhance gene expression in a host organism. These include, for example, elimination of: one or more sequences encoding spurious polyadenylation signals, one or more exon-intron splice site signals, one or more transposon-like repeats, and other such well-characterized sequences that may be deleterious to gene expression. The G-C content of the sequence may be adjusted to levels average for a given host organism, as calculated by reference to known genes expressed in the host cell. When possible, the sequence is modified to avoid one or more predicted hairpin secondary mRNA structures.

As used herein, the term “coding sequence” refers to a nucleotide sequence, which directly specifies the amino acid sequence of its (encoded) protein product. The boundaries of the coding sequence are generally determined by an open reading frame (hereinafter, “ORF”), which usually begins with an ATG start codon. The coding sequence typically includes DNA, cDNA, and recombinant nucleotide sequences.

As defined herein, the term “open reading frame” (hereinafter, “ORF”) means a nucleic acid or nucleic acid sequence (whether naturally occurring, non-naturally occurring, or synthetic) comprising an uninterrupted reading frame consisting of (i) an initiation codon, (ii) a series of two (2) or more codons representing amino acids, and (iii) a termination codon, the ORF being read (or translated) in the 5′ to 3′ direction.

The term “chromosomal integration” as used herein refers to a process where the donor DNA (polynucleotide of interest) is integrated into the Bacillus sp. chromosome. The homology arms flanking the linear donor DNA construct (linear donor DNA flanked by homology arms) will align with homologous regions of the Bacillus sp. chromosome. Subsequently, the sequence between the homology arms is replaced by the donor DNA (polynucleotide of interest) in a double crossover (i.e., homologous recombination).

“Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include, but are not limited to, promoters, translation leader sequences, 5′ untranslated sequences, 3′ untranslated sequences, introns, polyadenylation target sequences, RNA processing sites, effector binding sites, and stem-loop structures.

The term “promoter” as used herein refers to a nucleic acid sequence capable of controlling the expression of a coding sequence or functional RNA. In general, a coding sequence is located 3′ (downstream) to a promoter sequence. Promoters may be derived in their entirety from a native gene, or be composed of different elements derived from different promoters found in nature, or even comprise synthetic nucleic acid segments. It is understood by those skilled in the art that different promoters may direct the expression of a gene in different cell types, or at different stages of development, or in response to different environmental or physiological conditions. Promoters which cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity.

“Operably linked” is intended to mean a functional linkage between two or more elements. For example, an operable linkage between a polynucleotide of interest and a regulatory sequence (e.g., a promoter) is a functional link that allows for expression of the polynucleotide of interest (i.e., the polynucleotide of interest is under transcriptional control of the promoter). Operably linked elements may be contiguous or non-contiguous. Coding sequences (e.g., an ORF) can be operably linked to regulatory sequences in sense or antisense orientation. When used to refer to the joining of two protein coding regions, by operably linked is intended that the coding regions are in the same reading frame.

A nucleic acid is “operably linked” when it is placed into a functional relationship with another nucleic acid sequence. For example, DNA encoding a secretory leader (i.e., a signal peptide), is operably linked to DNA for a polypeptide if it is expressed as a pre-protein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

As used herein, “a functional promoter sequence controlling the expression of a gene of interest (or open reading frame thereof) linked to the gene of interest's protein coding sequence” refers to a promoter sequence which controls the transcription and translation of the coding sequence in Bacillus. For example, in certain embodiments, the present disclosure is directed to a polynucleotide comprising a 5′ promoter (or 5′ promoter region, or tandem 5′ promoters and the like), wherein the promoter region is operably linked to a nucleic acid sequence encoding a protein of interest. Thus, in certain embodiments, a functional promoter sequence controls the expression of a gene of interest encoding a protein of interest. In other embodiments, a functional promoter sequence controls the expression of a heterologous gene or an endogenous gene encoding a protein of interest in a Bacillus sp. cell.

The promoter sequence consists of proximal and more distal upstream elements, the latter elements often referred to as enhancers. An “enhancer” is a DNA sequence that can stimulate promoter activity, and may be an innate element of the promoter or a heterologous element inserted to enhance the level or tissue-specificity of a promoter.

The circular recombinant DNAs disclosed herein can be introduced into a Bacillus sp. Cell using any method known in the art.

As defined herein, the term “introducing”, as used in phrases such as “introducing into a bacterial cell” or “introducing into a Bacillus sp. cell” at least one recombinant DNA, polynucleotide, or a gene thereof, or a vector thereof, includes methods known in the art for introducing polynucleotides into a cell, including, but not limited to protoplast fusion, natural or artificial transformation (e.g., calcium chloride, electroporation, heat shock), transduction, transfection, conjugation and the like (e.g., see Ferrari et al., 1989).

“Introducing” is intended to mean presenting to the organism, such as a cell or organism, the circular recombinant DNAs disclosed herein, in such a manner that the component(s) gains access to the interior of a cell of the organism or to the cell itself. The methods and compositions do not depend on a particular method for introducing a sequence into an organism or cell, only that the circular recombinant DNAs disclosed herein gains access to the interior of at least one cell of the organism. Introducing includes reference to the incorporation of a nucleic acid into a Bacillus sp. cell where the nucleic acid may be incorporated into the genome of the cell, and includes reference to the transient (direct) provision of a nucleic acid to the cell.

Methods for introducing polynucleotides, expression cassettes, recombinant DNA into cells or organisms are known in the art including, but not limited to, natural competence (as described in WO2017/075195, WO2002/14490 and WO2008/7989), microinjection Crossway et al., (1986) Biotechniques 4:320-34 and U.S. Pat. No. 6,300,543), meristem transformation (U.S. Pat. No. 5,736,369), electroporation (Riggs et al., (1986) Proc. Natl. Acad. Sci. USA 83:5602-6), stable transformation methods, transient transformation methods, ballistic particle acceleration (particle bombardment) (U.S. Pat. Nos. 4,945,050; 5,879,918; 5,886,244; 5,932,782), whiskers mediated transformation (Ainley et al. 2013, Plant Biotechnology Journal 11:1126-1134; Shaheen A. and M. Arshad 2011 Properties and Applications of Silicon Carbide (2011), 345-358 Editor(s): Gerhardt, Rosario. Publisher: InTech, Rijeka, Croatia. CODEN: 69PQBP; ISBN: 978-953-307-201-2), Agrobacterium-mediated transformation (U.S. Pat. Nos. 5,563,055 and 5,981,840), direct gene transfer (Paszkowski et al., (1984) EMBO J 3:2717-22), viral-mediated introduction (U.S. Pat. Nos. 5,889,191, 5,889,190, 5,866,785, 5,589,367 and 5,316,931), transfection, transduction, cell-penetrating peptides, mesoporous silica nanoparticle (MSN)-mediated direct protein delivery, topical applications, sexual crossing, sexual breeding, and any combination thereof. Stable transformation is intended to mean that the nucleotide construct introduced into an organism integrates into a genome of the organism and is capable of being inherited by the progeny thereof. Transient transformation is intended to mean that a polynucleotide is introduced (directly or indirectly) into the organism and does not integrate into a genome of the organism or a polypeptide is introduced into an organism. Transient transformation indicates that the introduced composition is only temporarily expressed or present in the organism.

A variety of methods are available for identifying those cells with insertion into the genome at or near to the target site. Such methods can be viewed as directly analyzing a target sequence to detect any change in the target sequence, including but not limited to PCR methods, sequencing methods, nuclease digestion, Southern blots, and any combination thereof. See, for example, U.S. patent application Ser. No. 12/147,834, herein incorporated by reference to the extent necessary for the methods described herein. The method also comprises recovering an organism from the cell comprising a polynucleotide of interest integrated into its genome.

The term “genome”, a bacterial (host) cell “genome”, or a Bacillus (host) cell “genome” includes not only chromosomal DNA found within the nucleus, but organelle DNA found within subcellular components of the cell (extrachromosomal DNA).

As used herein, the terms “plasmid”, “vector” and “cassette” refer to extrachromosomal elements, often carrying genes which are typically not part of the central metabolism of the cell, and usually in the form of double-stranded DNA molecules. Such elements may be autonomously replicating sequences, genome integrating sequences, phage or nucleotide sequences, linear or circular, of a single-stranded or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

The term “vector” includes any nucleic acid that can be replicated (propagated) in cells and can carry new genes or DNA segments into cells. Vectors include viruses, bacteriophage, pro-viruses, plasmids, phagemids, transposons, and artificial chromosomes such as BACs (bacterial artificial chromosomes), and the like, that are “episomes” (i.e., replicate autonomously or can integrate into a chromosome of a host organism).

The term “expression cassette” and “expression vector” refer to a nucleic acid construct generated recombinantly or synthetically, with a series of specified nucleic acid elements that permit transcription of a particular nucleic acid in a cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter. In some embodiments, DNA constructs also include a series of specified nucleic acid elements that permit transcription of a particular nucleic acid in a target cell. In certain embodiments, a DNA construct of the disclosure comprises a selective marker and an inactivating chromosomal or gene or DNA segment as defined herein. Many prokaryotic expression vectors are commercially available and know to one skilled in the art. Selection of appropriate expression vectors is within the knowledge of one skilled in the art.

As used herein, a “targeting vector” is a vector that includes polynucleotide sequences that are homologous to a region in the chromosome of a host cell into which the targeting vector is transformed and that can drive homologous recombination at that region. For example, targeting vectors find use in introducing mutations into the chromosome of a host cell through homologous recombination. In some embodiments, the targeting vector comprises other non-homologous sequences, e.g., added to the ends (i.e., stuffer sequences or flanking sequences). The ends can be closed such that the targeting vector forms a closed circle, such as, for example, insertion into a vector. Selection and/or construction of appropriate vectors is well within the knowledge of those having skill in the art.

As used herein, the term “plasmid” refers to a circular double-stranded (ds) DNA construct used as a cloning vector, and which forms an extrachromosomal self-replicating genetic element in many bacteria and some eukaryotes. In some embodiments, plasmids become incorporated into the genome of the host cell.

Polynucleotides of interest are further described herein and include polynucleotides reflective of the commercial markets and interests of those involved in the production of enzymes (such as, but not limiting to, through fermentation of bacteria thereby producing the enzymes.

Polynucleotides of interest may also comprise antisense sequences complementary to at least a portion of the messenger RNA (mRNA) for a targeted gene sequence of interest. Antisense nucleotides are constructed to hybridize with the corresponding mRNA. Modifications of the antisense sequences may be made as long as the sequences hybridize to and interfere with expression of the corresponding mRNA. In this manner, antisense constructions having 70%, 80%, or 85% sequence identity to the corresponding antisense sequences may be used. Furthermore, portions of the antisense nucleotides may be used to disrupt the expression of the target gene. Generally, sequences of at least 50 nucleotides, 100 nucleotides, 200 nucleotides, or greater may be used.

In addition, the polynucleotide of interest may also be used in the sense orientation to suppress the expression of endogenous genes in organisms. Methods for suppressing gene expression in organisms using polynucleotides in the sense orientation are known in the art. The methods generally involve transforming an organism with a DNA construct comprising a promoter that drives expression in an organism operably linked to at least a portion of a nucleotide sequence that corresponds to the transcript of the endogenous gene. Typically, such a nucleotide sequence has substantial sequence identity to the sequence of the transcript of the endogenous gene, generally greater than about 65% sequence identity, about 85% sequence identity, or greater than about 95% sequence identity. See, U.S. Pat. Nos. 5,283,184 and 5,034,323; herein incorporated by reference.

A phenotypic marker is screenable or a selectable marker that includes visual markers and selectable markers whether it is a positive or negative selectable marker. Any phenotypic marker can be used. Specifically, a selectable or screenable marker comprises a DNA segment that allows one to identify, or select for or against a molecule or a cell that contains it, often under particular conditions. These markers can encode an activity, such as, but not limited to, production of RNA, peptide, or protein, or can provide a binding site for RNA, peptides, proteins, inorganic and organic compounds or compositions and the like.

The term “selectable marker” and “selectable marker-encoding nucleotide sequence” refers to a nucleotide sequence which is capable of expression in (host) cells and where expression of the selectable marker confers to cells containing the expressed gene the ability to grow in the presence of a corresponding selective agent or lack of an essential nutrient. In one aspect the selective marker refers to a nucleic acid (e.g., a gene) capable of expression in host cell which allows for ease of selection of those hosts containing the vector. Examples of such selectable markers include, but are not limited to, antimicrobials.

The term “selectable marker” includes genes that provide an indication that a host cell has taken up an incoming DNA of interest or some other reaction has occurred. Typically, selectable markers are genes that confer antimicrobial resistance or a metabolic advantage on the host cell to allow cells containing the exogenous DNA to be distinguished from cells that have not received any exogenous sequence during the transformation.

A “residing selectable marker” is one that is located on the chromosome of the microorganism to be transformed. A residing selectable marker encodes a gene that is different from the selectable marker on the transforming DNA construct. Selective markers are well known to those of skill in the art. As indicated above, the marker can be an antimicrobial resistance marker (e.g., amp^{R}, phleo^{R}, spec^{R}, kan^{R}, ery^{R}, tet^{R}, cmp^{R} and neo^{R} (see e.g., Guerot-Fleury, 1995; Palmeros et al., 2000; and Trieu-Cuot et al., 1983). In some embodiments, the present invention provides a chloramphenicol resistance gene (e.g., the gene present on pC194, as well as the resistance gene present in the Bacillus licheniformis genome). This resistance gene is particularly useful in the present invention, as well as in embodiments involving chromosomal amplification of chromosomally integrated cassettes and integrative plasmids (See e.g., Albertini and Galizzi, 1985; Stahl and Ferrari, 1984). Other markers useful in accordance with the invention include, but are not limited to auxotrophic markers, such as serine, lysine, tryptophan; and detection markers, such as β-galactosidase.

Polynucleotides of interest includes genes that can be stacked or used in combination with other traits.

As used herein, the terms “polypeptide” and “protein” are used interchangeably, and refer to polymers of any length comprising amino acid residues linked by peptide bonds. The conventional one (1) letter or three (3) letter codes for amino acid residues are used herein. The polypeptide may be linear or branched, it may comprise modified amino acids, and it may be interrupted by non-amino acids. The term polypeptide also encompasses an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

The term “protein of interest” or “POI” refers to a polypeptide of interest that is desired to be expressed in a modified Bacillus (daughter) cell. Thus, as used herein, a POI may be an enzyme, a substrate-binding protein, a surface-active protein, a structural protein, a receptor protein, an antibody and the like

As used herein, a “gene of interest” or “GOI” refers a nucleic acid sequence (e.g., a polynucleotide, a gene or an ORF) which encodes a POI. A “gene of interest” encoding a “protein of interest” may be a naturally occurring gene, a mutated gene or a synthetic gene.

In certain embodiments, a gene of interest of the instant disclosure encodes a commercially relevant industrial protein of interest, such as an enzyme (e.g., a acetyl esterases, aminopeptidases, amylases, arabinases, arabinofuranosidases, carbonic anhydrases, carboxypeptidases, catalases, cellulases, chitinases, chymosins, cutinases, deoxyribonucleases, epimerases, esterases, α-galactosidases, β-galactosidases, α-glucanases, glucan lysases, endo-β-glucanases, glucoamylases, glucose oxidases, α-glucosidases, β-glucosidases, glucuronidases, glycosyl hydrolases, hemicellulases, hexose oxidases, hydrolases, invertases, isomerases, laccases, lipases, lyases, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phytases, polygalacturonases, proteases, peptidases, rhamno-galacturonases, ribonucleases, transferases, transport proteins, transglutaminases, xylanases, hexose oxidases, and combinations thereof).

A “mutation” refers to any change or alteration in a nucleic acid sequence. Several types of mutations exist, including point mutations, deletion mutations, silent mutations, frame shift mutations, splicing mutations and the like. Mutations may be performed specifically (e.g., via site directed mutagenesis) or randomly (e.g., via chemical agents, passage through repair minus bacterial strains).

A “mutated gene” is a gene that has been altered through human intervention. Such a “mutated gene” has a sequence that differs from the sequence of the corresponding non-mutated gene by at least one nucleotide addition, deletion, or substitution. In certain embodiments of the disclosure, the mutated gene comprises an alteration that results from a guide polynucleotide/Cas protein system as disclosed herein. A mutated cell or organism is a cell or organism comprising a mutated gene.

As used herein, a “targeted mutation” is a mutation in a gene (referred to as the target gene), including a native gene, that was made by altering a target sequence within the target gene using any method known to one skilled in the art, including a method involving a guided Cas protein system. Where the Cas protein is a cas endonuclease, a guide polynucleotide/Cas endonuclease induced targeted mutation can occur in a nucleotide sequence that is located within or outside a genomic target site that is recognized and cleaved by the Cas endonuclease.

As used herein, in the context of a polypeptide or a sequence thereof, the term “substitution” means the replacement (i.e., substitution) of one amino acid with another amino acid.

As defined herein, an “endogenous gene” refers to a gene in its natural location in the genome of an organism.

As used herein, “heterologous” in reference to a polynucleotide or polypeptide sequence is a sequence that originates from a foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide. As used herein, unless otherwise specified, a chimeric polynucleotide comprises a coding sequence operably linked to a transcription initiation region that is heterologous to the coding sequence.

As defined herein, a “heterologous” gene, a “non-endogenous” gene, or a “foreign” gene refer to a gene (or ORF) not normally found in the host organism, but that is introduced into the host organism by gene transfer. As used herein, the term “foreign” gene(s) comprise native genes (or ORFs) inserted into a non-native organism and/or chimeric genes inserted into a native or non-native organism.

As defined herein, a “heterologous” nucleic acid construct or a “heterologous” nucleic acid sequence has a portion of the sequence which is not native to the cell in which it is expressed.

As defined herein, a “heterologous control sequence”, refers to a gene expression control sequence (e.g., a promoter or enhancer) which does not function in nature to regulate (control) the expression of the gene of interest. Generally, heterologous nucleic acid sequences are not endogenous (native) to the cell, or a part of the genome in which they are present, and have been added to the cell, by infection, transfection, transformation, microinjection, electroporation, and the like. A “heterologous” nucleic acid construct may contain a control sequence/DNA coding (ORF) sequence combination that is the same as, or different, from a control sequence/DNA coding sequence combination found in the native host cell.

As used herein, the terms “signal sequence” and “signal peptide” refer to a sequence of amino acid residues that may participate in the secretion or direct transport of a mature protein or precursor form of a protein. The signal sequence is typically located N-terminal to the precursor or mature protein sequence. The signal sequence may be endogenous or exogenous. A signal sequence is normally absent from the mature protein. A signal sequence is typically cleaved from the protein by a signal peptidase after the protein is transported.

The term “derived” encompasses the terms “originated” “obtained,” “obtainable,” and “created,” and generally indicates that one specified material or composition finds its origin in another specified material or composition, or has features that can be described with reference to the another specified material or composition.

As used herein, a “flanking sequence” refers to any sequence that is either upstream or downstream of the sequence being discussed (e.g., for genes A-B-C, gene B is flanked by the A and C gene sequences). In certain embodiments, the incoming sequence is flanked by a homology arm on each side. In some embodiments, a flanking sequence is present on only a single side (either 3′ or 5′), while in other embodiments, it is on each side of the sequence being flanked. The sequence of each homology arm is homologous to a sequence in the Bacillus genome (such as the Bacillus chromosome).

As used herein, the term “stuffer sequence” refers to any extra DNA that flanks homology arms (typically vector sequences). However, the term encompasses any non-homologous DNA sequence. Not to be limited by any theory, a stuffer sequence provides a non-critical target for a cell to initiate DNA uptake.

Sequence identity” or “identity” in the context of nucleic acid or polypeptide sequences refers to the nucleic acid bases or amino acid residues in two sequences that are the same when aligned for maximum correspondence over a specified comparison window.

The term “percentage of sequence identity” refers to the value determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the results by 100 to yield the percentage of sequence identity. Useful examples of percent sequence identities include, but are not limited to, 50%, 55%, 60%, 65%, 70%, 75%, 80%, 85%, 90% or 95%, or any integer percentage from 50% to 100%. These identities can be determined using any of the programs described herein.

Sequence alignments and percent identity or similarity calculations may be determined using a variety of comparison methods designed to detect homologous sequences including, but not limited to, the MegAlign™ program of the LASERGENE bioinformatics computing suite (DNASTAR Inc., Madison, Wis.). Within the context of this application it will be understood that where sequence analysis software is used for analysis, that the results of the analysis will be based on the “default values” of the program referenced, unless otherwise specified. As used herein “default values” will mean any set of values or parameters that originally load with the software when first initialized.

The “Clustal V method of alignment” corresponds to the alignment method labeled Clustal V (described by Higgins and Sharp, (1989) CABIOS 5:151-153; Higgins et al., (1992) Comput Appl Biosci 8:189-191) and found in the MegAlign™ program of the LASERGENE bioinformatics computing suite (DNASTAR Inc., Madison, Wis.). For multiple alignments, the default values correspond to GAP PENALTY=10 and GAP LENGTH PENALTY=10. Default parameters for pairwise alignments and calculation of percent identity of protein sequences using the Clustal method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4. After alignment of the sequences using the Clustal V program, it is possible to obtain a “percent identity” by viewing the “sequence distances” table in the same program.

The “Clustal W method of alignment” corresponds to the alignment method labeled Clustal W (described by Higgins and Sharp, (1989) CABIOS 5:151-153; Higgins et al., (1992) Comput Appl Biosci 8:189-191) and found in the MegAlign™ v 6.1 program of the LASERGENE bioinformatics computing suite (DNASTAR Inc., Madison, Wis.). Default parameters for multiple alignment (GAP PENALTY=10, GAP LENGTH PENALTY=0.2, Delay Divergen Seqs (%)=30, DNA Transition Weight=0.5, Protein Weight Matrix=Gonnet Series, DNA Weight Matrix=IUB). After alignment of the sequences using the Clustal W program, it is possible to obtain a “percent identity” by viewing the “sequence distances” table in the same program.

Unless otherwise stated, sequence identity/similarity values provided herein refer to the value obtained using GAP Version 10 (GCG, Accelrys, San Diego, Calif.) using the following parameters: % identity and % similarity for a nucleotide sequence using a gap creation penalty weight of 50 and a gap length extension penalty weight of 3, and the nwsgapdna.cmp scoring matrix; % identity and % similarity for an amino acid sequence using a GAP creation penalty weight of 8 and a gap length extension penalty of 2, and the BLOSUM62 scoring matrix (Henikoff and Henikoff, (1989) Proc. Natl. Acad. Sci. USA 89:10915). GAP uses the algorithm of Needleman and Wunsch, (1970) J Mol Biol 48:443-53, to find an alignment of two complete sequences that maximizes the number of matches and minimizes the number of gaps. GAP considers all possible alignments and gap positions and creates the alignment with the largest number of matched bases and the fewest gaps, using a gap creation penalty and a gap extension penalty in units of matched bases.

“BLAST” is a searching algorithm provided by the National Center for Biotechnology Information (NCBI) used to find regions of similarity between biological sequences. The program compares nucleotide or protein sequences to sequence databases and calculates the statistical significance of matches to identify sequences having sufficient similarity to a query sequence such that the similarity would not be predicted to have occurred randomly. BLAST reports the identified sequences and their local alignment to the query sequence.

It is well understood by one skilled in the art that many levels of sequence identity are useful in identifying polypeptides from other species or modified naturally or synthetically wherein such polypeptides have the same or similar function or activity. Useful examples of percent identities include, but are not limited to, 50%, 55%, 60%, 65%, 70%, 75%, 80%, 85%, 90% or 95%, or any integer percentage from 50% to 100%. Indeed, any integer amino acid identity from 50% to 100% may be useful in describing the present disclosure, such as 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%.

“Translation leader sequence” refers to a polynucleotide sequence located between the promoter sequence of a gene and the coding sequence. The translation leader sequence is present in the mRNA upstream of the translation start sequence. The translation leader sequence may affect processing of the primary transcript to mRNA, mRNA stability or translation efficiency. Examples of translation leader sequences have been described (e.g., Turner and Foster, (1995) Mol Biotechnol 3:225-236).

“3′ non-coding sequences”, “transcription terminator” or “termination sequences” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht et al., (1989) Plant Cell 1:671-680.

As used herein, “RNA transcript” refers to the product resulting from RNA polymerase-catalyzed transcription of a DNA sequence. When the RNA transcript is a perfect complimentary copy of the DNA sequence, it is referred to as the primary transcript or pre-mRNA. A RNA transcript is referred to as the mature RNA or mRNA when it is a RNA sequence derived from post-transcriptional processing of the primary transcript pre-mRNA. “Messenger RNA” or “mRNA” refers to the RNA that is without introns and that can be translated into protein by the cell. “cDNA” refers to a DNA that is complementary to, and synthesized from, an mRNA template using the enzyme reverse transcriptase. The cDNA can be single-stranded or converted into double-stranded form using the Klenow fragment of DNA polymerase I. “Sense” RNA refers to RNA transcript that includes the mRNA and can be translated into protein within a cell or in vitro. “Antisense RNA” refers to an RNA transcript that is complementary to all or part of a target primary transcript or mRNA, and that blocks the expression of a target gene (see, e.g., U.S. Pat. No. 5,107,065). The complementarity of an antisense RNA may be with any part of the specific gene transcript, i.e., at the 5′ non-coding sequence, 3′ non-coding sequence, introns, or the coding sequence. “Functional RNA” refers to antisense RNA, ribozyme RNA, or other RNA that may not be translated but yet has an effect on cellular processes. The terms “complement” and “reverse complement” are used interchangeably herein with respect to mRNA transcripts, and are meant to define the antisense RNA of the message.

“Mature” protein refers to a post-translationally processed polypeptide (i.e., one from which any pre- or propeptides present in the primary translation product have been removed). “Precursor” protein refers to the primary product of translation of mRNA (i.e., with pre- and propeptides still present). Pre- and propeptides may be but are not limited to intracellular localization signals.

Proteins may be altered in various ways including amino acid substitutions, deletions, truncations, and insertions. Methods for such manipulations are generally known. For example, amino acid sequence variants of the protein(s) can be prepared by mutations in the DNA. Methods for mutagenesis and nucleotide sequence alterations include, for example, Kunkel, (1985) Proc. Natl. Acad. Sci. USA 82:488-92; Kunkel et al., (1987) Meth Enzymol 154:367-82; U.S. Pat. No. 4,873,192; Walker and Gaastra, eds. (1983) Techniques in Molecular Biology (MacMillan Publishing Company, New York) and the references cited therein. Guidance regarding amino acid substitutions not likely to affect biological activity of the protein is found, for example, in the model of Dayhoff et al., (1978) Atlas of Protein Sequence and Structure (Natl Biomed Res Found, Washington, D.C.). Conservative substitutions, such as exchanging one amino acid with another having similar properties, may be preferable. Conservative deletions, insertions, and amino acid substitutions are not expected to produce radical changes in the characteristics of the protein, and the effect of any substitution, deletion, insertion, or combination thereof can be evaluated by routine screening assays. Assays for double-strand-break-inducing activity are known and generally measure the overall activity and specificity of the agent on DNA substrates containing target sites.

Standard DNA isolation, purification, molecular cloning, vector construction, and verification/characterization methods are well established, see, for example Sambrook et al., (1989) Molecular Cloning: A Laboratory Manual, (Cold Spring Harbor Laboratory Press, NY). Vectors and constructs include circular plasmids, and linear polynucleotides, comprising a polynucleotide of interest and optionally other components including linkers, adapters, regulatory or analysis. In some examples a recognition site and/or target site can be contained within an intron, coding sequence, 5′ UTRs, 3′ UTRs, and/or regulatory regions.

The meaning of abbreviations is as follows: “sec” means second(s), “min” means minute(s), “h” means hour(s), “d” means day(s), “μL” means microliter(s), “mL” means milliliter(s), “L” means liter(s), “μM” means micromolar, “mM” means millimolar, “M” means molar, “mmol” means millimole(s), “pmole” mean micromole(s), “g” means gram(s), “μg” means microgram(s), “ng” means nanogram(s), “U” means unit(s), “bp” means base pair(s) and “kb” means kilobase(s).

Non-limiting examples of compositions and methods disclosed herein are as follows:
1. A method for integrating a gene of interest into a target site on the genome of a Bacillus sp. cell without the integration of a selectable marker into said genome, the method comprising simultaneously introducing at least a first circular recombinant DNA construct and a second circular recombinant DNA construct into a Bacillus sp. cell, wherein said first circular recombinant DNA construct comprises a donor DNA sequence comprising a gene of interest encoding a protein of interest and comprises a DNA sequence encoding a guide RNA, wherein said second circular recombinant DNA construct comprises a Cas9 endonuclease DNA sequence operably linked to a constitutive promoter, wherein said Cas9 endonuclease DNA sequence encodes a Cas9 that introduces a double-strand break at or near a target site in the genome of said Bacillus sp. cell.
2. The method of embodiment 1, wherein the donor DNA sequence is flanked by two homology arms, one upstream arm (5′ HR1) and one downstream arm (3′ HR2) wherein each homology arm is between 70 nucleotides and 600 nucleotides, between 100 and 600 nucleotides, between 200 and 600 nucleotides, between 300 and 600 nucleotides, between 400 and 600 nucleotides, between 500 and 600 nucleotides, or up to 600 nucleotides in length, and comprises sequence homology to a targeted genomic locus of said Bacillus sp. cell.
2b. The method of embodiment 1, wherein the donor DNA sequence is flanked by an upstream homology arm (HR1) and a downstream homology arm (HR2) of 600 bps or less.
3. The method of embodiment 1, 2 or 2b, further comprising growing progeny cells from said Bacillus sp. cell and selecting a Bacillus sp. progeny cell that has the gene of interest stably integrated in its genome.
4. The method of embodiment 3, wherein the first circular recombinant DNA construct and second circular recombinant DNA construct comprise a selectable marker that is not integrated into the genome of said Bacillus sp. progeny cell.
4b. The method of embodiment 4, wherein said selectable marker is not stably integrated into the genome of said Bacillus sp. progeny cell.
5. The method of embodiment 1 or 2, having a frequency of integration of the gene of interest gene into said genome that is at least about 2, 3, 4, 5, 6, 7, 8, 9, 10 up to 11 fold higher when compared to the frequency of integration of a control method comprising introducing into a Bacillus sp. cell a linear recombinant DNA construct comprising said donor DNA sequence flanked by said at least one homology arm, and a circular recombinant DNA construct comprising said DNA sequence encoding said guide RNA and comprising said Cas9 endonuclease DNA sequence operably linked to a constitutive promoter.
5b. The method of embodiment 1 or 2, having a frequency of integration of the gene of interest gene that is at least about 2, 3, 4, 5, 6, 7, 8, 9, 10 up to 11 fold higher when compared to the frequency of integration of a control method comprising introducing into a Bacillus sp. cell a linear recombinant DNA construct comprising said donor DNA sequence flanked by an upstream (HR1) and downstream homology arm (HR2) of 1000 bps, and a circular recombinant DNA construct comprising said DNA sequence encoding said guide RNA and said Cas9 endonuclease DNA sequence operably linked to a constitutive promoter.
6. The method of embodiment 1 or 2, wherein the first circular recombinant DNA construct and/or the second circular recombinant DNA construct comprise an autonomous replicating sequence.
7. The method of embodiment 6, wherein said first circular recombinant DNA construct comprising a donor DNA sequence comprising a gene of interest and comprising a DNA sequence encoding a guide RNA is a low copy plasmid.
8. The method of embodiment 1 or 2, wherein the Bacillus sp. cell is selected from the group consisting of Bacillus subtilis, Bacillus licheniformis, Bacillus lentus, Bacillus brevis, Bacillus stearothermophilus, Bacillus alkalophilus, Bacillus amyloliquefaciens, Bacillus clausii, Bacillus. halodurans, Bacillus. megaterium, Bacillus coagulans, Bacillus circulans, Bacillus lautus, and Bacillus thuringiensis.
9. The method of embodiment 1 or 2, wherein the first and second circular recombinant DNA constructs are simultaneously introduced into the Bacillus sp. cell via one mean selected from the group consisting of protoplast fusion, natural or artificial transformation, electroporation, heat-shock, transduction, transfection, conjugation, phage delivery, mating, natural competence, induced competence, and any combination thereof.
10. A modified Bacillus sp. cell, comprising at least a first circular recombinant DNA construct and a second circular recombinant DNA construct, wherein said first circular recombinant DNA construct comprises a DNA sequence encoding a guide RNA and a donor DNA sequence comprising a gene of interest, wherein said guide RNA comprises a sequence complementary to a target site sequence on a chromosome or episome of said Bacillus sp. cell, wherein said second circular recombinant DNA construct comprises a Cas9 endonuclease DNA sequence operably linked to a constitutive promoter, wherein said Cas9 endonuclease DNA sequence encodes a Cas9 endonuclease that can form a RNA-guided endonuclease (RGEN), wherein said RGEN can bind to, and optionally cleave, all or part of the target site sequence.
11. The modified Bacillus sp. cell of embodiment 10, wherein said gene of interest is integrated into the genome of said Bacillus sp. cell.
12. A method for integrating multiple copies of a gene expression cassette into the genome of a Bacillus sp. cell without the integration of a selectable marker into said genome, the method comprising simultaneously introducing at least a first circular recombinant DNA construct and a second circular recombinant DNA construct into a Bacillus sp. cell, wherein said first circular recombinant DNA construct comprises a donor DNA sequence comprising multiple copies of a gene expression cassette, wherein each gene expression cassette comprising the same gene of interest and comprising a DNA sequence encoding a guide RNA, wherein said second circular recombinant DNA construct comprises a Cas9 endonuclease DNA sequence operably linked to a constitutive promoter, wherein said Cas9 endonuclease DNA sequence encodes a Cas9 that introduces a double-strand break at or near a target site in the genome of said Bacillus sp. cell.
13. The method of embodiment 12, wherein the donor DNA is flanked by two homology arms, one upstream arm (5′ HR1) and one downstream arm (3′ HR2) wherein each homology arm is between 70 nucleotides and 600 nucleotides, between 100 and 600 nucleotides, between 200 and 600 nucleotides, between 300 and 600 nucleotides, between 400 and 600 nucleotides, between 500 and 600 nucleotides, up to 600 nucleotides in length, and comprises sequence homology to a targeted genomic locus of said Bacillus sp. cell.
14. The method of embodiment 12, wherein the donor DNA sequence is flanked by an upstream homology arm (HR1) and a downstream homology arm (HR2) of 600 bps or less.
15. The method of embodiment 12, further comprising growing progeny cells from said Bacillus sp. cell and selecting a Bacillus sp. progeny cell that has the multiple copies of said gene expression cassette stably integrated in its genome.
16. The method of embodiment 12, wherein the multiple copies of said gene expression cassette are selected from the group consisting of 2 copies, 3 copies, 4 copies, 5 copies, 6 copies, 7 copies, 8 copies, 9 copies and up to 10 copies.

### EXAMPLES

The disclosed disclosure is further defined in the following Examples. It should be understood that these Examples, while indicating certain preferred aspects of the disclosure, are given by way of illustration only. From the above discussion and these Examples, one skilled in the art can ascertain the essential characteristics of this disclosure, and without departing from the spirit and scope thereof, can make various changes and modifications of the disclosure to adapt it to various uses and conditions.

### Example 1

Construction of a Circular Recombinant DNA Construct Expressing Cas9 Endonuclease (pRF694)

A synthetic polynucleotide encoding the Cas9 protein from S. pyogenes (SEQ ID NO NO: 1), comprising an N-terminal nuclear localization sequence (NLS; “APKKKRKV”; SEQ ID NO: 2), a C-terminal NLS (“KKKKLK”; SEQ ID NO: 3) and a deca-histidine tag (“HHHHHHHHHH”; SEQ ID NO: 4), was operably linked to the constitutive aprE promoter from B. subtilis and amplified using Q5 DNA polymerase (NEB) per manufacturer's instructions with the forward (SEQ ID NO: 5) and reverse (SEQ ID NO: 6) primer pair set. The backbone (SEQ ID NO: 7) of plasmid pKB320 (SEQ ID NO: 8) was amplified using Q5 DNA polymerase (NEB) per manufacturer's instructions with the forward (SEQ ID NO: 9) and reverse (SEQ ID NO: 10) primer pair set.

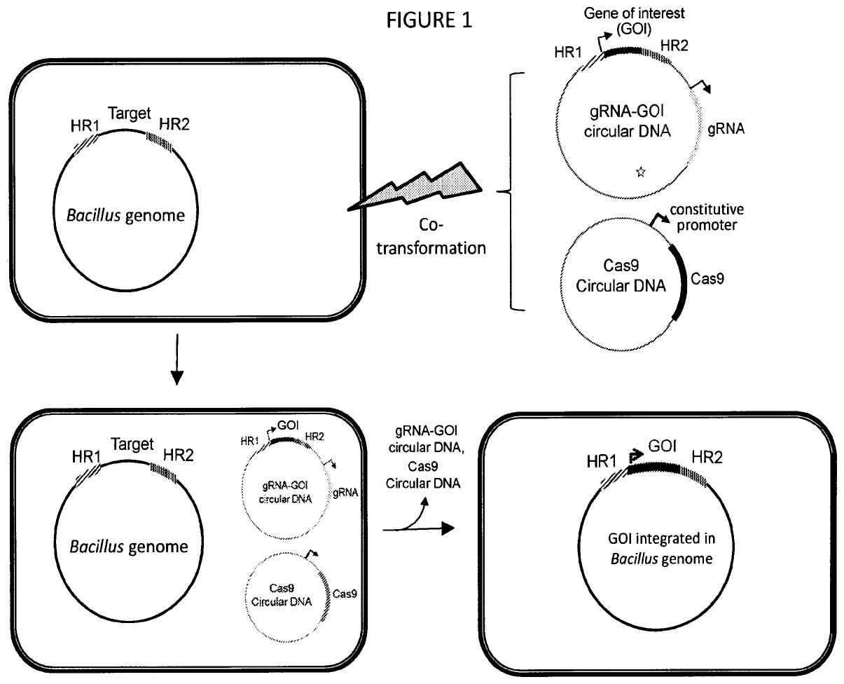

The PCR products were purified using Zymo clean and concentrate 5 columns per manufacturer's instructions. Subsequently, the PCR products were assembled using prolonged overlap extension PCR (POE-PCR) with Q5 Polymerase (NEB) mixing the two fragments at equimolar ratio. The POE-PCR reactions were cycled: 98° C. for 5 seconds, 64° C. for 10 seconds, 72° C. for 4 minutes and 15 seconds for 30 cycles. Five μl of the POE-PCR (DNA) was transformed into Top10 E. coli (Invitrogen) per manufacturer's instructions and selected on lysogeny (L) Broth (Miller recipe; 1% (w/v) Tryptone, 0.5% Yeast extract (w/v), 1% NaCl (w/v)), containing 50 μg/ml kanamycin sulfate and solidified with 1.5% Agar. Colonies were allowed to grow for 18 hours at 37° C. Colonies were picked and plasmid DNA prepared using Qiaprep DNA miniprep kit per manufacturer's instructions and eluted in 55 μl of ddH₂O. The plasmid DNA (pRF694, illustrated as second recombinant DNA construct in FIG. 1) was Sanger sequenced to verify correct assembly, using the sequencing primers (SEQ ID NOs: 11-19) set.

### Example 2

Construction of a Circular Recombinant DNA Construct (pWS534) for Integration of a V49 Protease Gene Expression Cassette (GOI) at the skf Gene Locus in Bacillus subtilis

A circular recombinant DNA construct (pWS534, illustrated as first circular recombinant DNA construct in FIG. 1) was constructed comprising a DNA sequences comprising a guide RNA expression cassette (SEQ ID NO: 20), V49 protease gene expression cassette (SEQ ID NO: 21), Bacillus subtilis skf gene up-stream homology arm (SEQ ID NO: 22), Bacillus subtilis skf gene down-stream homology arm (SEQ ID NO: 23), chloramphenicol resistant gene (cm^{R}) (SEQ ID NO: 24), pAMb1 replicon (SEQ ID NO: 25), and yeast 2-micron replicon and ura3 gene (SEQ ID NO: 26). The V49 protease gene is a protease variant from Bacillus clausii.

All DNA fragments were PCR-amplified using primers with 40-50 bp of overlap using Q5 DNA polymerase. The gRNA expression cassette (SEQ ID NO: 20) containing a spac promoter (SEQ ID NO: 27), DNA encoding a gRNA targeting the Bacillus subtilis skf gene (SEQ ID NO: 28), and terminator (SEQ ID NO: 29) was PCR-amplified from pRF787 template DNA with primers ws831 (SEQ ID: 30) and ws832 (SEQ ID NO: 31). The DNA fragment containing a Bacillus subtilis skf gene upstream homology, V49 protease gene expression cassette, and Bacillus subtilis skf downstream homology arm was PCR-amplified from pSCF3 template DNA with primers ws833 (SEQ ID NO: 32) and ws834 (SEQ ID NO: 33). The DNA fragment containing a chloramphenicol resistant gene (cm^{R}) and pAMb1 replicon were PCR-amplified from pAMBR2 template DNA with primers ws835 (SEQ ID NO: 34) and ws777 (SEQ ID NO: 35). The yeast 2-micron replicon and ura3 gene was PCR-amplified from pWS528 template DNA with primers ws778 (SEQ ID NO: 36) and ws836 (SEQ ID NO: 37). All PCR fragments were purified by using QIAGEN PCR purification kit or Gel extraction kit (QIAGEN, Inc). The purified PCR fragments were transformed in S. cerevisiae by using Frozen-EZ Yeast Transformation II™ (Zymo Research, Inc). 50 μl of S. cerevisiae competent cells was mixed with 0.1-0.2 μg DNA of each PCR fragment. Add 500 μl EZ3 solution, mix thoroughly, incubate at 30° C. for 45 minutes, spread 50-100 μl of the above transformation mixture on an SC-ura plate, and incubate the plates at 30° C. for 2-4 days to allow for growth of transformants. Plasmid DNA was prepared from 1 ml of S. cerevisiae grown in SC-ura medium by using Zymo Yeast Plasmid kit. The prepared plasmid was confirmed by PCR with primers ws823 (SEQ ID NO: 38) and ws824 (SEQ ID NO: 39), and DNA sequencing with primers ws894 (SEQ ID NO: 40), ws895 (SEQ ID: 41) and ws896 (SEQ ID: 42). The resulting plasmid was named as pWS534.

To construct the pSCF3 plasmid, a DNA sequence comprising a gene of interest, a synthetic DNA fragment containing the rrnlp2 promoter (SEQ ID NO: 43)-aprE signal sequence (SEQ ID NO: 44)-pro sequence (SEQ ID NO: 45)-V49 protease (SEQ ID NO: 46)-terminator (SEQ ID NO: 47) expression cassette was amplified with primers 1133 (SEQ ID: 48) and 1134 (SEQ ID: 49). The 600 bp Bacillus subtilis skf upstream homology arm was amplified from Bacillus subtilis genomic DNA using primers 1131 (SEQ ID: 50) and 1165 (SEQ ID: 51), the 600 bp Bacillus subtilis skf downstream homology was amplified from Bacillus subtilis genomic DNA with primers 1132 (SEQ ID: 52) and 1166 (SEQ ID: 53), the pRS423 plasmid backbone was amplified with oligonucleotides 1164 (SEQ ID NO: 54) and 1163 (SEQ ID NO: 55). All fragments had appropriate overlap for assembly by gap repair. Plasmid was assembled in S288C Saccharomyces cerevisiae cells by transforming with 50 ng of plasmid fragment and equimolar amounts of each additional fragments. Strains containing the gap repaired plasmid were selected for histidine prototrophy. Expression cassette on plasmids were sequenced using oligonucleotides 1169 (SEQ ID NO: 56), 1170 (SEQ ID NO: 57), 1171 (SEQ ID NO: 58). The resulting plasmid was named pSCF3.

An E. coli/Gram-positive shuttle vector pAMBR2 (SEQ ID NO: 59) was constructed by cloning a ori322 replicon and ampicillin resistant gene (amp^{R}) gene from pBR322 [Gene. 1988, 70: 399-403] and chloramphenicol resistant gene (cm^{R}) from Staphylococcus aureus plasmid pC194 [J. Bacteriol. 1982, 150:815-825] on the pAMb1 plasmid backbone from Enterococcus faecalis [J. Bacteriol. 1984, 157:445-453].

A E. coli/yeast shuttle vector pWS528 (SEQ ID NO: 60) was constructed by combining a yeast 2-micron replicon and ura3 gene from a yeast plasmid pRS426 (ATCC® 77107™), spac promoter [Yansura & Henner, 1984, Proc. Natl. Acad. Sci. USA 81:439-443], gRNA synthesized by gblock (IDT Inc.), and chloramphenicol resistant gene (cm^{R})-pAMb1 replicon from pAMBR2 by gap-repair in Saccharomyces cerevisiae.

### Example 3

Integration of a Protease Variant Expression Cassette (GOI) at the Skf Locus in Bacillus subtilis Using a Linear Recombinant DNA Construct (Control Method)

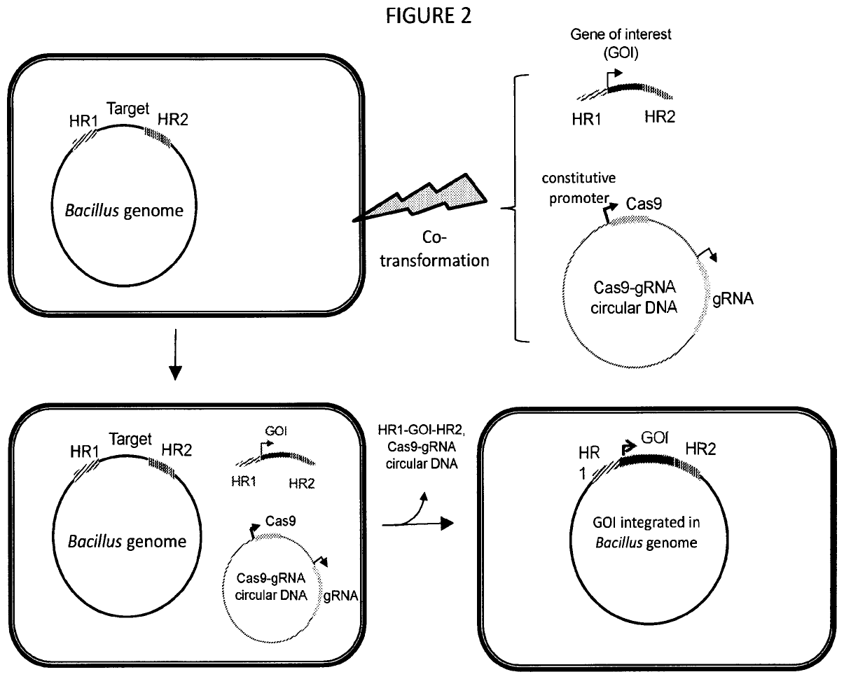

This example describes the use of a linear recombinant DNA construct comprising a donor DNA encoding a protease variant (gene of interest), flanked by two homology arms (HR1 and HR2 of 1000 bps in length or less, and a circular recombinant DNA construct comprising a DNA sequence encoding a guideRNA and a Cas9 endonuclease DNA sequence operably linked to a constitutive promoter, in simultaneously introducing the first and second recombinant DNAs to enable efficient introduction of a large gene of interest (e.g., DNA encoding he protease variant) into a Bacillus host cell. The gene of interest was integrated at the skf gene locus in Bacillus subtilis as described below. (FIG. 2)

The correctly assembled plasmid, pRF694 (SEQ ID NO: 61), as described above in example 1, was used to assemble the intermediate plasmid, pRF747 (SEQ ID NO: 62). The construction of plasmid pRF747 was created by cloning an interrupted synthetic gRNA cassette into the NcoI/SalI sites of plasmid pRF694. This cassette was produced synthetically by IDT and contains the Bacillus subtilis narKp promoter (SEQ ID NO: 63), a synthetic double terminator (SEQ ID NO: 64), the E. coli rpsL gene (SEQ ID NO: 65), the DNA encoding the Cas9 endonuclease recognition domain (SEQ ID NO: 66), and the lambda phage TO terminator (SEQ ID NO: 67). The DNA fragment containing the gRNA expression cassette was cloned into pRF694 using standard molecular biology techniques generating plasmid pRF747, generating an E. coli-B. subtilis shuttle plasmid containing a Cas9 expression cassette and a gRNA expression cassette.

The intermediate plasmid, pRF747 was used to assemble the plasmid for the introduction of the expression cassettes into the skf locus of B. subtilis. More particularly, the skfC gene (SEQ ID NO: 68) in the skf locus of B. subtilis contains a Cas9 target site (SEQ ID NO: 69). The target site was converted into a DNA sequence encoding a variable targeting (VT) domain (SEQ ID NO: 70) by removing the PAM sequence (last three nucleotides of SEQ ID NO: 71). The DNA sequence encoding the VT domain was operably fused to the DNA sequence encoding the Cas9 Endonuclease Recognition domain such that when transcribed by RNA polymerase in the cell, it produces a functional gRNA (SEQ ID NO: 72). The DNA encoding the gRNA (SEQ ID NO: 73) was operably linked to a promoter operable in Bacillus sp. cells (e.g., the rrnl promoter from B. subtilis; SEQ ID NO: 74) and a terminator operable in Bacillus sp. cells (e.g., the t0 terminator of lambda phage; SEQ ID NO: 67), such that the promoter was positioned 5′ of the DNA encoding the gRNA and the terminator was positioned 3′ of the DNA encoding the gRNA, to create a gRNA expression cassette (SEQ ID NO: 75).

Plasmid pRF776 (SEQ ID NO: 76), targeting the skf gene of B. subtilis was created by amplifying plasmid pRF747 (SEQ ID NO: 62), using Q5 according to the manufacturer's instructions and the forward (SEQ ID NO: 77) and reverse (SEQ ID NO: 78) primer pairs.

These primers amplify the entire plasmid (pRF747) except for the variable targeting region of the gRNA creating a fragment in which the 5′ and 3′ ends overlap and containing the skf variable targeting domain. This PCR product was used for an intramolecular assembly reaction using NEBuilder (New England Biolabs) per the manufacturer's instructions, to create plasmid pRF776 (SEQ ID NO: 76), generating an E. coli-B. subtilis shuttle plasmid containing a Cas9 expression cassette and a gRNA expression cassette that encoding a gRNA targeting skf.

In the present example, a protease expression cassette was integrated into Bacillus subtilis genome. More specifically, these expression cassettes contained the DNA sequence homologous to flanking region 5′ of the skf genes (SEQ ID NO: 79) operably fused to the DNA sequence encoding a promoter operable in B. subtilis cells (e.g., the native Bacillus subtilis rrnl promoter which was operably fused to the DNA sequence encoding a protease variant mature gene, operably fused to the DNA sequence encoding the Bacillus amyloliquefaciens apr terminator (SEQ ID NO: 80) such that the promoter was positioned 5′ of the DNA encoding the mature gene and the terminator was positioned 3′ of the DNA encoding the mature gene. The expression cassette described above was operably fused to the DNA sequence homologous to the flanking region 3′ of the skf genes (SEQ ID NO: 81).

Thus, in the present example, parental B. subtilis cells containing the B. subtilis comK gene (SEQ ID NO: 82) introduced at the amyE locus using the PxylA inducible promoter for expression, were grown overnight at 37° C. and 250 RPM in 15 ml of L broth (1% w·v⁻¹ Tryptone, 0.5% Yeast extract w·v⁻¹, 1% NaCl w·v⁻¹), in a 125 ml baffled flask. The overnight culture was diluted to 0.2 (OD₆₀₀ units) in 10 ml fresh L broth in a one hundred twenty-five (125) ml baffle flask. Cells were grown until the culture reached 0.9 (OD₆₀₀ units) at 37° C. (250 RPM). D-xylose was added to 0.3% (w/v) from a 30% (w/v) stock. Cells were grown for an additional 2.5 hours at 37° C. (250 RPM) and pelleted at 1700×g for 7 minutes. The cells were resuspended in one fourth (Y4) volume of original culture using the spent medium. 100 μl of concentrated cells were mixed with approximately 1 μg of the variant protease expression cassette and the pRF776 plasmid (SEQ ID NO: 76) described above, which was amplified using rolling circle amplification (Syngis) for 18 hours according to the manufacturer's instructions. Cell/DNA transformation mixes were plated onto L-broth (miller) containing ten (10) μg/mL kanamycin, 1.6% (w/v) skim milk and solidified with 1.5% (w/v) agar. Colonies were allowed to form at 37° C. Colonies that grew on L agar containing kanamycin and skim milk and produced a visible clearing zone in the area adjacent to the colonies, indicative of proteolytic activity, were picked and streaked onto agar plates containing 1.6% (w/v) skim milk.

Integration efficiency for protease variant expression cassettes integrated at the skf locus in parental B. subtilis strains using the plasmid pRF776 (SEQ ID NO: 76) and linear expression cassettes was 0 (zero) percent (see also Table 1, Example 5).

### Example 4

Integration of a Protease Variant Expression Cassette (GOI) at the aprEyhfN Locus in Bacillus subtilis Using a Linear Recombinant DNA Construct (Control Method)

This example describes the use of a linear recombinant DNA construct comprising a donor DNA sequence encoding a protease variant (gene of interest), flanked by two homology arms (HR1 and HR2 of 1000 bps in length or less), and a circular recombinant DNA construct comprising a DNA sequence encoding a guideRNA and a Cas9 endonuclease DNA sequence operably linked to a constitutive promoter, in simultaneously introducing the first and second recombinant DNAs to enable efficient introduction of a large gene of interest (e.g., DNA encoding the protease variant) into a Bacillus host cell. The gene of interest (a protease variant) was integrated at the aprEyhfN locus in Bacillus subtilis strain as described below (FIG. 2).

The correctly assembled plasmid, pRF694 (SEQ ID NO: 61), as described above in example 1, was used to assemble the intermediate plasmid, pRF748 (SEQ ID NO: 83). The construction of plasmid pRF748 was created by cloning an interrupted synthetic gRNA cassette into the NcoI/SalI sites of plasmid pRF694. This cassette was produced synthetically by IDT and contains the B. subtilis rrnl promoter (SEQ ID NO:74), a synthetic double terminator (SEQ ID NO: 64), the E. coli rpsL gene (SEQ ID NO: 65), the DNA encoding the Cas9 endonuclease recognition domain (SEQ ID NO: 66), and the lambda phage TO terminator (SEQ ID NO: 67). The DNA fragment containing the gRNA expression cassette was assembled into pRF694 using standard molecular biology techniques generating plasmid pRF748, generating an E. coli-B. subtilis shuttle plasmid containing a Cas9 expression cassette and a gRNA expression cassette.

The intermediate plasmid, pRF748 (SEQ ID NO: 83) was used to assemble the plasmid for the introduction of the expression cassettes into the aprEyhfN locus of B. subtilis. More particularly, the yhfN gene (SEQ ID NO: 84) in the aprE locus of B. subtilis contains a Cas9 target site (SEQ ID NO: 85). The target site was converted into a DNA sequence encoding a variable targeting (VT) domain (SEQ ID NO: 86) by removing the PAM sequence (last three nucleotides of SEQ ID NO: 87). The DNA sequence encoding the VT domain (SEQ ID NO: 86) was operably fused to the DNA sequence encoding the Cas9 Endonuclease Recognition domain (CER; SEQ ID NO: 66) such that when transcribed by RNA polymerase in the cell, it produced a functional gRNA (SEQ ID NO: 88). The DNA encoding the gRNA (SEQ ID NO: 89) was operably linked to a promoter operable in Bacillus sp. cells (e.g., the rrnl promoter from B. subtilis; SEQ ID NO: 74) and a terminator operable in Bacillus sp. cells (e.g., the t0 terminator of lambda phage; SEQ ID NO: 67), such that the promoter was positioned 5′ of the DNA encoding the gRNA and the terminator was positioned 3′ of the DNA encoding the gRNA, to create a gRNA expression cassette (SEQ ID NO: 90).

Plasmid pRF793 (SEQ ID NO: 91), targeting the yhfN gene (SEQ ID NO: 85) of B. subtilis was created by amplifying plasmid pRF748 (SEQ ID NO: 83), using Q5 according to the manufacturer's instructions and the forward (SEQ ID NO: 92) and reverse (SEQ ID NO: 93) primer pairs.

In the present example, a protease expression cassette was integrated into B. subtilis genome. More specifically, these expression cassettes contained the DNA sequence homologous to flanking region 5′ of the yhfN gene (SEQ ID NO: 94) operably fused to the DNA sequence encoding a promoter operable in B. subtilis cells (e.g., the native B. subtilis rrnl promoter SEQ ID NO: 74) which was operably fused to the DNA sequence encoding a protease variant mature gene, operably fused to the DNA sequence encoding the B. amyloliquefaciens apr terminator (SEQ ID NO: 80) such that the promoter was positioned 5′ of the DNA encoding the mature gene and the terminator was positioned 3′ of the DNA encoding the mature gene. The expression cassette described above was operably fused to the DNA sequence homologous to the flanking region 3′ of the yhfN gene (SEQ ID NO:95).

Thus, in the present example, parental B. subtilis cells containing the B. subtilis comK gene (SEQ ID N0:82) introduced at the amyE locus using the PxylA inducible promoter for expression, were grown overnight at 37° C. and 250 RPM in fifteen (15) ml of L broth (1% w·v⁻¹ Tryptone, 0.5% Yeast extract w·v⁻¹, 1% NaCl w·v⁻¹), in a 125 ml baffled flask. The overnight culture was diluted to 0.2 (OD₆₀₀ units) in 10 ml fresh L broth in a 125 ml baffle flask. Cells were grown until the culture reached 0.9 (OD₆₀₀ units) at 37° C. (250 RPM). D-xylose was added to 0.3% (w/v) from a 30% (w/v) stock. Cells were grown for an additional 2.5 hours at 37° C. (250 RPM) and pelleted at 1700×g for 7 minutes. The cells were resuspended in one fourth (Y4) volume of original culture using the spent medium. 100 μl of concentrated cells were mixed with approximately one (1) μg of the variant protease expression cassette and the pRF793 plasmid (SEQ ID NO: 91) described above, which was amplified using rolling circle amplification (Syngis) for 18 hours according to the manufacturer's instructions. Cell/DNA transformation mixes were plated onto L-broth (miller) containing 10 μg/mL kanamycin, 1.6% (w/v) skim milk and solidified with 1.5% (w/v) agar. Colonies were allowed to form at 37° C. Colonies that grew on L agar containing kanamycin and skim milk and produced a visible clearing zone in the area adjacent to the colonies, indicative of proteolytic activity, were picked and streaked onto agar plates containing 1.6% (w/v) skim milk.

Integration efficiency for protease variant expression cassettes integrated at the aprE locus in parental B. subtilis strains using the plasmid pRF793 (SEQ ID NO: 91) and linear expression cassettes was 6 percent (see also Table 1, Example 5).

### Example 5

Integration of a V49 Protease Gene Expression Cassette (GOI) at the Skf Gene Locus in Bacillus subtilis by Simultaneous Introduction of Two Circular Recombinant DNA Constructs

This example describes an integration of a V49 protease gene expression cassette (Gene of interest, GOI) at the skf gene locus in Bacillus subtilis amyE::comK by the dual circular recombinant DNA method (FIG. 1). B. subtilis amyE::comK competent cells were transformed with rolling circle amplification (RCA) mixtures of both a first circular recombinant DNA (plasmid pWS534 (see example 2) and a second recombinant DNA construct expressing Cas9 endonuclease (plasmid pRF694, Example 1) as describe below.

To make competent cells of Bacillus subtilis amyE::comK, colonies from a fresh plate were inoculated in 10 ml of LB medium in 125 ml flask, and incubated with 250 rpm shaking at 37° C. overnight. Overnight culture was diluted to OD₆₀₀=0.2 in 10 ml LB medium in 125 ml flask, and incubated with 250 rpm shaking at 37° C. to OD₆₀₀=0.9. 34 μl of 33% D-xylose stock solution was added to 0.1% final concentration, and grew with 250 rpm shaking at 37° C. for 2 hours. 10 ml competent cells were mixed with 4 ml 50% glycerol, dispensed as 0.6 ml aliquots, and stored at −80° C. until ready for use.

To transform with two plasmids pRF694 (kanamycin^{R}) and pWS534 (chloramphenicol^{R}) simultaneously into competent cells, firstly rolling circle amplification (RCA) mixtures of both pRF694 and pWS534 were prepared by using the TruePrime™ RCA kit (Lucigen Inc). Secondly, 100 μl of competent cells were mixed with 20 μl RCA mixtures of two plasmids in an eppendorf tube, and incubated at 37° C. for 1.5-2 hrs with 250 rpm shaking. The cells were plated on the LB medium with a supplement with kanamycin (final conc. 20 μg/ml) and chloramphenicol (final conc. 5 μg/ml), and incubated at 37 C overnight. After incubation for 20 hrs at 37° C., three kanamycin (kan^{R}) and chloramphenicol (cm^{R}) resistant colonies were obtained.

Three kan^{R} and cm^{R} colonies were tested for the correct integration of a V49 protease gene expression cassette (SEQ ID NO: 21) at the skf locus in B. subtilis by colony PCR with a forward primer WS823 (SEQ ID NO: 38) and a reverse primer WS824 (SEQ ID NO: 39) at the flanking region of the skf locus.

Surprisingly, two out of three colonies (representing a 67% frequency of integration) showed the expected size of PCR band (2.9 kbp).

A summary table of the integration frequency for expression cassettes comprising a gene of interest integrated by either the dual circular recombinant DNA method (with one circular DNA comprising the donor DNA) compared to a control method comprising a linear recombinant DNA comprising the donor DNA is shown in Table 1.

TABLE 1
Gene of interest (GOI) integration frequency

| | | | Frequency |
|---|---|---|---|
| | Length of | | of |
| | homology | Strain/ | integration |
| GOI integration method | arm (bp) | Target::GOI | (%) |
| Simultaneous introduction | 600 | B. subtilis/ | 67 |
| of two recombinant DNAs | | skf::GOI | |
| (circular donor DNA and | | | |
| circular Cas9 construct) | | | |
| Simultaneous introduction | 1000 | B. subtilis/ | 0 |
| of linear recombinant DNA | | skf::GOI | |
| comprising donor DNA | | | |
| (linear donor DNA) and | 1000 | B. subtilis/ | 6 |
| circular Cas9 construct | | aprEyhfN::GOI | |

Table 1 clearly illustrates the surprising observation that the integration of a gene of interest (encoding a protein of interest) into a Bacillus sp. host genome by the dual circular recombinant DNA method described herein is highly efficient when compared to integration of a gene of interest by a control method comprising a linear donor DNA flanked by 1000 bp homology arms, and a circular Cas9 cassette. Both control experiments directing the gene of interest to be integrated at two different sites in the genome of a Bacillus sp. cell using a linear donor DNA resulted in a low frequency of integration indicating that the location of integration is independent from the observed increase in frequency of integrations when a dual circular recombinant system is used.

What is claimed:

1. A method for integrating a gene of interest into a target site on the genome of a Bacillus sp. cell without the integration of a selectable marker into said genome, the method comprising simultaneously introducing at least a first circular recombinant DNA construct and a second circular recombinant DNA construct into a Bacillus sp. cell, wherein said first circular recombinant DNA construct comprises a donor DNA sequence comprising a gene of interest and a DNA sequence encoding a guide RNA, wherein said second circular recombinant DNA construct comprises a Cas9 endonuclease DNA sequence operably linked to a constitutive promoter, wherein said Cas9 endonuclease DNA sequence encodes a Cas9 that introduces a double-strand break at or near a target site in the genome of said Bacillus sp. cell.

2. The method of claim 1, wherein the donor DNA sequence is flanked by two homology arms, one upstream homology arm (5′ HR1) and one downstream homology arm (3′ HR2) wherein each homology arm is between 70 nucleotides and 600 nucleotides, between 100 and 600 nucleotides, between 200 and 600 nucleotides, between 300 and 600 nucleotides, between 400 and 600 nucleotides, between 500 and 600 nucleotides, or up to 600 nucleotides in length, and comprises sequence homology to said target site on the genome of the Bacillus sp. cell.

3. The method of claim 1 or 2, further comprising growing progeny cells from said Bacillus sp. cell and selecting a Bacillus sp. progeny cell that has the gene of interest stably integrated in its genome.

4. The method of claim 3, wherein the first circular recombinant DNA construct and second circular recombinant DNA construct comprise a selectable marker that is not integrated into the genome of said Bacillus sp. progeny cell.

5. The method of claim 4, wherein said selectable marker is not stably integrated into the genome of said Bacillus sp. progeny cell.

6. The method of claim 1 or 2, having a frequency of integration of the gene of interest gene into the genome of a Bacillus sp. cell that is at least about 2, 3, 4, 5, 6, 7, 8, 9, 10 up to 11 fold higher when compared to the frequency of integration of a control method comprising introducing into a Bacillus sp. cell a linear recombinant DNA construct comprising said donor DNA sequence flanked by an upstream (HR1) and downstream homology arm (HR2) of 1000 bps, and a circular recombinant DNA construct comprising said DNA sequence encoding said guide RNA and said Cas9 endonuclease DNA sequence operably linked to a constitutive promoter.

7. The method of claim 1 or 2, wherein the first circular recombinant DNA construct and/or the second circular recombinant DNA construct comprise an autonomous replicating sequence.

8. The method of claim 6, wherein said first circular recombinant DNA construct comprising a donor DNA sequence comprising a gene of interest and a DNA sequence encoding a guide RNA is a low copy plasmid.

9. The method of claim 1 or 2, wherein the Bacillus sp. cell is selected from the group consisting of Bacillus subtilis, Bacillus licheniformis, Bacillus lentus, Bacillus brevis, Bacillus stearothermophilus, Bacillus alkalophilus, Bacillus amyloliquefaciens, Bacillus clausii, Bacillus halodurans, Bacillus megaterium, Bacillus coagulans, Bacillus circulans, Bacillus lautus, and Bacillus thuringiensis.

10. The method of claim 1 or 2, wherein the first and second circular recombinant DNA constructs are simultaneously introduced into the Bacillus sp. cell via one mean selected from the group consisting of protoplast fusion, natural or artificial transformation, electroporation, heat-shock, transduction, transfection, conjugation, phage delivery, mating, natural competence, induced competence, and any combination thereof.
